(19) United States
(12) Patent Application Publication
(10) Pub. No.: US 2024/0376497 A1
(43) Pub. Date: Nov. 14, 2024

(54) FRATAXIN GENE THERAPY

(71) Applicant: Astellas Gene Therapies, Inc., South San Francisco, CA (US)

(72) Inventors: Dwaipayan SEN, San Bruno, CA (US); John T. GRAY, Marblehead, MA (US); Joshua C. CHANG, Foster City, CA (US)

(21) Appl. No.: 18/692,672

(22) PCT Filed: Sep. 16, 2022
(86) PCT No.: PCT/US2022/076563

§ 371 Date: Mar. 15, 2024

Related U.S. Application Data
(60) Provisional application No. 63/341,737, filed on May 13, 2022.
(60) Provisional application No. 63/331,441, filed on Apr. 15, 2022.
(60) Provisional application No. 63/245,666, filed on Sep. 17, 2021.

(51) Int. Cl. C12N 15/86; A61K 48/00; A61P 25/00; C07K 14/47

(52) U.S. Cl. CPC C12N 15/86; A61K 48/0058; A61P 25/00; C07K 14/47; C12N 2750/14143; C12N 2830/008

(57) ABSTRACT

The invention provides compositions and methods for stimulating the expression of the human frataxin gene. The compositions described herein can be used, for instance, to produce genes and RNA equivalents optimized for expression in a particular cell type. The compositions and methods that can be used for treating Frederich ataxia. Using the compositions and methods of the disclosure, a patient (e.g., a mammalian patient, such as a human patient) having Frederich ataxia may be administered a plasmid (e.g., a viral vector) that contains a human frataxin gene (hFXN) or an RNA equivalent thereof.

### SEQUENCE LISTING

The instant application contains a Sequence Listing which has been submitted electronically in XML format and is hereby incorporated by reference in its entirety. Said XML copy, created on Sep. 9, 2022, is named 51037-060WO4_Sequence_Listing_9_8_22_ST26.XML and is 23,124 bytes in size.

### FIELD OF THE INVENTION

The invention relates to the field of nucleic acid biotechnology and provides, for instance, compositions and methods for production of codon-optimized nucleic acids for enhancing gene expression in a target cell or tissue

### BACKGROUND OF THE INVENTION

Friedreich ataxia is the most common autosomal recessive inherited movement disorder, with 6,000 Americans diagnosed with this disease, and a prevalence of approximately 15,000 to 20,000 patients diagnosed worldwide. The clinical manifestations of Friedreich ataxia are the result of deficiency of the frataxin protein, with 95% of cases resulting from mutations that cause a GAA repeat expansion. Frataxin is a mitochondrial iron-binding protein that is ubiquitously expressed and is critical to the function of the heart, cerebellum, and spinal cord, including dorsal root ganglia neurons. The phenotype of Friedreich ataxia includes degeneration and demyelination of the spinocerebellar dorsal root ganglion neurons, leading to progressive weakness, spasticity, and sensory loss; and most Friedreich patients are wheelchair-bound by the age of 20 (Dun and Brice, Curr Opin Neurol (2000) 13:407-413). Furthermore, most patients with Friedreich ataxia develop cardiac abnormalities (e.g., left ventricular hypertrophy), resulting in death from heart failure by the age of about 30 to 40 in 60% of patients. The development of gene therapies for the treatment of Friedreich ataxia have been hindered by the difficulty associated with achieving expression of therapeutically effective amounts of frataxin in affected tissues, and currently no approved disease-modifying treatments exist. There remains a need for a set of compositions and methods that address these hindrances.

### SUMMARY OF THE INVENTION

The present disclosure provides compositions and methods that can be used for treating Frederich ataxia. Using the compositions and methods of the disclosure, a patient (e.g., a mammalian patient, such as a human patient) having Frederich ataxia may be administered a plasmid (e.g., a viral vector) that contains a human frataxin gene (hFXN) or an RNA equivalent thereof.

In one aspect, the disclosure provides a DNA polynucleotide encoding hFXN, or an RNA equivalent thereof, wherein the polynucleotide has a nucleic acid sequence that is at least 95% (e.g., 95%, 96%, 97%, 98%, or 99%) identical to the nucleic acid sequence of SEQ ID NO: 1.

In another aspect, the disclosure provides a vector including the composition of the foregoing aspect, wherein the vector is a plasmid, a DNA vector, an RNA vector, a virion, or a viral vector. For example, in some embodiments, the vector is a viral vector. In some embodiments, the viral vector is an adeno-associated virus (AAV).

In some embodiments of the foregoing aspect, the polynucleotide is operably linked to a muscle specific promoter, optionally wherein the promoter is positioned 5′ to the polynucleotide.

In some embodiments, the vector further includes a polyadenylation site (pA), optionally wherein the pA is positioned 3′ to the polynucleotide.

In some embodiments, the vector further includes an intron, optionally wherein the intron is positioned 3′ to the promoter and 5′ to the polynucleotide.

In some embodiments, the AAV further comprises two inverted terminal repeats (ITRs), wherein the two ITRs comprise a first ITR (ITR1) and a second ITR (ITR2), wherein ITR1 is positioned 5′ to the polynucleotide and ITR2 is positioned 3′ to the polynucleotide to form a cassette comprising the structure ITR1-hFXN-ITR2.

In some embodiments, the length of the nucleic acid between ITR1 and ITR2 is from about 3.7 Kb to about 4.3 Kb (e.g., about 3.8 Kb to about 4.2 Kb or about 3.9 Kb to about 4.1 Kb). For example, in some embodiments, the length of the nucleic acid between ITR1 and ITR2 is from about 3.8 Kb to about 4.2 Kb. In some embodiments, the length of the nucleic acid between ITR1 and ITR2 is from about 3.9 Kb to about 4.1 Kb. In some embodiments, the length of the nucleic acid between ITR1 and ITR2 is about 4.0 Kb.

In another aspect, the disclosure provides a plasmid encoding the viral vector of the foregoing aspect.

In another aspect, the disclosure provides a nucleic acid molecule including: an ITR1; a hFXN or an RNA equivalent thereof; and an ITR2; wherein the components are operably linked to each other in a 5′-to-3′ direction as: ITR1-hFXN-ITR2; and wherein the length of the nucleic acid between ITR1 and ITR2 is from about 3.7 Kb to about 4.3 Kb (e.g., about 3.8 Kb to about 4.2 Kb or about 3.9 Kb to about 4.1 Kb). For example, in some embodiments, the length of the nucleic acid between ITR1 and ITR2 is from about 3.8 Kb to about 4.2 Kb. In some embodiments, the length of the nucleic acid between ITR1 and ITR2 is from about 3.9 Kb to about 4.1 Kb. In some embodiments, the length of the nucleic acid between ITR1 and ITR2 is about 4.0 Kb.

In some embodiments of any of the foregoing aspects, the length of the nucleic acids between and including ITR1 and ITR2 is about 3.9 Kb to about 4.7 Kb (e.g., about 4.0 Kb to about 4.6 Kb, about 4.1 Kb to about 4.5 Kb, about 4.2 Kb to about 4.4 Kb, or about 4.3 Kb). For example, in some embodiments, the length of the nucleic acids between and including ITR1 and ITR2 is about 4.0 Kb to about 4.6 Kb. In some embodiments, the length of the nucleic acids between and including ITR1 and ITR2 is about 4.1 Kb to about 4.5 Kb. In some embodiments, the length of the nucleic acids between and including ITR1 and ITR2 is about 4.2 Kb to about 4.4 Kb. In some embodiments, the length of the nucleic acids between and including ITR1 and ITR2 is about 4.3 Kb.

In some embodiments, the nucleic acid molecule further includes: a eukaryotic promoter (P_{Euk}), wherein the components are operably linked to each other in a 5′-to-3′ direction as: ITR1-P_{Euk}-hFXN-ITR2.

In some embodiments, the P_{Euk} is a muscle specific promoter.

In some embodiments of any of the foregoing aspects, the muscle specific promoter is a phosphoglycerate kinase (PGK) promoter, a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3, a cytomegalovirus promoter, or a chicken-β-actin promoter. For example, in some embodiments, the muscle specific promoter is a PGK promoter.

In some embodiments of any of the foregoing aspects, the PGK promoter has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid of sequence of SEQ ID NO: 2. For example, in some embodiments, the PGK promoter has a nucleic acid sequence that is at least 90% identical to the nucleic acid of sequence of SEQ ID NO: 2. In some embodiments, the PGK promoter has a nucleic acid sequence that is at least 95% identical to the nucleic acid of sequence of SEQ ID NO: 2, optionally wherein the PGK promoter has a nucleic acid sequence that is at least 96%, 97%, 98%, or 99% identical to the nucleic acid sequence of SEQ ID NO: 2. In some embodiments, the PGK promoter has a nucleic acid of SEQ ID NO: 2.

In some embodiments, the nucleic acid molecule further includes: a pA, wherein the components are operably linked to each other in a 5′-to-3′ direction as: ITR1-P_{Euk}-hFXN-pA-ITR2.

In some embodiments of any of the foregoing aspects, the pA site includes the simian virus 40 (SV40) late polyadenylation site, the SV40 early polyadenylation site, the human β-globin polyadenylation site, or the bovine growth hormone polyadenylation site. For example, in some embodiments, the pA site includes the SV40 late polyadenylation site.

In some embodiments, the nucleic acid molecule further includes: an intron, wherein the components are operably linked to each other in a 5′-to-3′ direction as: ITR1-P_{Euk}-intron-hFXN-pA-ITR2.

In some embodiments of any of the foregoing aspects, the intron is an SV40 intron.

In some embodiments, the hFXN, or an RNA equivalent thereof, encodes a protein that is at least 85% (e.g., 85%, 86%, 87%, 88%, 89%, 90%, 91%, 92%, 93%, 94%, 95%, 96%, 97%, 98%, or 99%) identical to the amino acid sequence of SEQ ID NO: 3. For example, in some embodiments, the hFXN, or an RNA equivalent thereof, encodes a protein that is at least 90% identical to the amino acid sequence of SEQ ID NO: 3, optionally wherein the hFXN, or an RNA equivalent thereof, encodes a protein that is at least 95%, 96%, 97%, 98%, or 99% identical to the amino acid sequence of SEQ ID NO: 3. In some embodiments, the hFXN, or an RNA equivalent thereof, encodes a protein having the amino acid sequence of SEQ ID NO: 3.

In some embodiments of any of the foregoing aspects, the hFXN, or an RNA equivalent thereof, has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 1. For example, in some embodiments, the hFXN, or an RNA equivalent thereof, has a nucleic acid sequence that is at least 86% identical to the nucleic acid sequence of SEQ ID NO: 1. In some embodiments, the hFXN, or an RNA equivalent thereof, has a nucleic acid sequence that is at least 87% identical to the nucleic acid sequence of SEQ ID NO: 1. In some embodiments, the hFXN, or an RNA equivalent thereof, has a nucleic acid sequence that is at least 88% identical to the nucleic acid sequence of SEQ ID NO: 1. In some embodiments, the hFXN, or an RNA equivalent thereof, has a nucleic acid sequence that is at least 89% identical to the nucleic acid sequence of SEQ ID NO: 1. In some embodiments, the hFXN, or an RNA equivalent thereof, has a nucleic acid sequence that is at least 90% identical to the nucleic acid sequence of SEQ ID NO: 1. In some embodiments, the hFXN, or an RNA equivalent thereof, has a nucleic acid sequence that is at least 91% identical to the nucleic acid sequence of SEQ ID NO: 1. In some embodiments, the hFXN, or an RNA equivalent thereof, has a nucleic acid sequence that is at least 92% identical to the nucleic acid sequence of SEQ ID NO: 1. In some embodiments, the hFXN, or an RNA equivalent thereof, has a nucleic acid sequence that is at least 93% identical to the nucleic acid sequence of SEQ ID NO: 1. In some embodiments, the hFXN, or an RNA equivalent thereof, has a nucleic acid sequence that is at least 94% identical to the nucleic acid sequence of SEQ ID NO: 1. In some embodiments, the hFXN, or an RNA equivalent thereof, has a nucleic acid sequence that is at least 95% identical to the nucleic acid sequence of SEQ ID NO: 1. In some embodiments, the hFXN, or an RNA equivalent thereof, has a nucleic acid sequence that is at least 96% identical to the nucleic acid sequence of SEQ ID NO: 1. In some embodiments, the hFXN, or an RNA equivalent thereof, has a nucleic acid sequence that is at least 97% identical to the nucleic acid sequence of SEQ ID NO: 1. In some embodiments, the hFXN, or an RNA equivalent thereof, has a nucleic acid sequence that is at least 98% identical to the nucleic acid sequence of SEQ ID NO: 1. In some embodiments, the hFXN, or an RNA equivalent thereof, has a nucleic acid sequence that is at least 99% identical to the nucleic acid sequence of SEQ ID NO: 1. In some embodiments, the hFXN, or an RNA equivalent thereof, has the nucleic acid sequence of SEQ ID NO: 1.

In another aspect, the disclosure provides a vector including the composition or the nucleic acid molecule of any of the foregoing aspects, wherein the vector is a plasmid, a DNA vector, an RNA vector, a virion, or a viral vector. For example, in some embodiments, the vector is a viral vector.

In some embodiments of any of the foregoing aspects, the viral vector is selected from the group consisting of an AAV, an adenovirus, a lentivirus, a retrovirus, a poxvirus, a baculovirus, a herpes simplex virus, a vaccinia virus, and a synthetic virus. For example, in some embodiments, the viral vector is an AAV. In some embodiments, the AAV is an AAV1, AAV2, AAV3, AAV4, AAV5, AAV6, AAV7, AAV8, AAV9, AAVrh10, or AAVrh74 serotype. In some embodiments, the viral vector is a pseudotyped AAV. In some embodiments, the pseudotyped AAV is AAV2/8 or AAV2/9, optionally wherein the pseudotyped AAV is AAV2/8.

In some embodiments of any of the foregoing aspects, ITR1 and/or ITR2 is a parvoviral ITR. For example, in some embodiments, the parvoviral ITR is an AAV ITR. In some embodiments, the AAV ITR is an AAV serotype 2 ITR.

In some embodiments of any of the foregoing aspects, the AAV includes a recombinant capsid protein.

In some embodiments of any of the foregoing aspects, the vector has a nucleic acid sequence that is at least 85% e.g., 85%, 86%, 87%, 88%, 89%, 90%, 91%, 92%, 93%, 94%, 95%, 96%, 97%, 98%, or 99%) identical to the nucleic acid sequence of SEQ ID NO: 4. For example, in some embodiments, the vector has a nucleic acid sequence that is at least 90% identical to the nucleic acid sequence of SEQ ID NO: 4. In some embodiments, the vector has a nucleic acid sequence that is at least 95% identical to the nucleic acid sequence of SEQ ID NO: 4, optionally wherein the vector has a nucleic acid sequence that is at least 96%, 97%, 98%, or 99% identical to the nucleic acid sequence of SEQ ID NO: 4. In some embodiments, the vector has a nucleic acid of SEQ ID NO: 4.

In another aspect, the disclosure provides a plasmid encoding the viral vector of any one of the foregoing aspects.

In some embodiments of the foregoing aspect, the plasmid further includes one or more spacers (SS), wherein the one or more SS is positioned 5′ to ITR1 and/or 3′ to ITR2. For example, in some embodiments, the plasmid includes two SS, wherein the two SS include a first spacer (SS1) and a second spacer (SS2), wherein SS1 is positioned 5′ to ITR1 and the SS2 is positioned 3′ to ITR2.

In some embodiments, the one or more SS does not include an open reading frame that is greater than 100 amino acids in length.

In some embodiments, the one or more SS does not include prokaryotic transcription factor binding sites.

In some embodiments of any of the foregoing aspects, SS1 is about 1.0 Kb to about 5.0 Kb (e.g., 1.5 Kb to about 4.5 Kb, about 2.0 Kb to about 4.0 Kb, or about 3.0 Kb) in length. For example, in some embodiments, SS1 is about 2.0 Kb to about 5.0 Kb in length.

In some embodiments of any of the foregoing aspects, SS2 is about 1.0 Kb to about 5.0 Kb (e.g., 1.5 Kb to about 4.5 Kb, about 2.0 Kb to about 4.0 Kb, or about 3.0 Kb) in length. For example, in some embodiments, SS2 is about 2.0 Kb to about 5.0 Kb in length.

In some embodiments, the plasmid further includes a prokaryotic promoter operably linked to a selectable marker gene positioned 5′ to the one or more SS positioned 5′ to ITR1 or positioned 3′ to the one or more SS positioned 3′ to ITR2. For example, in some embodiments, the selectable marker gene is an antibiotic resistance gene.

In some embodiments, the plasmid further includes a prokaryotic origin of replication positioned 5′ to the one or more SS positioned 5′ to ITR1 and/or positioned 3′ to the one or more SS positioned 3′ to ITR2.

In another aspect, the disclosure provides a pharmaceutical composition including the composition, the nucleic acid molecule, the vector, or the plasmid of any of the foregoing aspects and a pharmaceutically acceptable carrier, diluent, or excipient.

In another aspect, the disclosure provides a method of treating Friedreich Ataxia in a human patient in need thereof, the method including administering to the patient a therapeutically effective amount of the composition, the nucleic acid molecule, the vector, the plasmid, or the pharmaceutical composition of any of the foregoing aspects.

In another aspect, the disclosure provides a method of increasing frataxin expression in a human patient diagnosed as having Friedreich Ataxia, the method including administering to the patient a therapeutically effective amount of the composition, the nucleic acid molecule, the vector, the plasmid, or the pharmaceutical composition of any of the foregoing aspects.

In some embodiments, the patient is from 3 years of age to 17 years (e.g., 4 years of age to 16 years, 5 years of age to 15 years, 6 years of age to 14 years, 7 years of age to 13 years, 8 years of age to 12 years, 9 years of age to 11 years, or 10 years) of age.

In some embodiments, upon administering the composition, the nucleic acid molecule, the vector, the plasmid, or the pharmaceutical composition of any of the foregoing aspects to the patient, the patient displays a change in whole blood frataxin levels, optionally wherein the patient displays the change in whole blood frataxin levels by about 12 weeks after administration.

In some embodiments, upon administering the composition, the nucleic acid molecule, the vector, the plasmid, or the pharmaceutical composition of any of the foregoing aspects to the patient, the patient displays a reduction in Total Friedreich Ataxia Rating Scale (FARS) Score, optionally wherein the patient displays the reduction in Total FARS Score by about 12 weeks after administration.

In another aspect, the disclosure provides a kit including the composition, the nucleic acid molecule, the vector, the plasmid, or the pharmaceutical composition of any of the foregoing aspects; and a package insert, wherein the package insert instructs a user of the kit to administer the composition or vector to a human patient diagnosed as having Friedreich Ataxia.

### BRIEF DESCRIPTION OF THE DRAWINGS

FIG. 1 is a schematic depicting the probability of nucleic acid variations in residue positions 1-270, respectively, of the gene encoding human frataxin (FXN) variant 1 (H.FXN.WT). Using the Integrated DNA Technologies (IDT) and GENEWIZ (GeneWiz) codon optimization tools, residue optimizations were compared across databases and residue positions. Whilst residues 1-270 are depicted herein, codon optimization was performed across all residues of the human FXN variant 1 gene, including residues 1-633. The sequence at the top indicates the most frequent nucleic acid at the respective residue position across the datasets. Arrows indicate a residue chosen for modification in a codon-optimized human FXN variant 1 construct (abbreviated interchangeably as H.FXN.ATX.Co or hFXNco). For example, residue 12 denotes that wild-type cytosine (C) was changed to guanine (G).

FIG. 2 is a map of an exemplary pseudotyped adeno-associated virus (AAV) 2/8 (AAV2/8) viral vector for the expression of a codon-optimized human FXN variant 1 gene (hFXNco). From left to right, the shaded arrows represent a plasmid containing a nucleic acid molecule including from 5′-to-3′ a first spacer, a first inverted terminal repeat (ITR1), a human phosphoglycerate kinase (hPGK) promoter, an hFXNco, a simian virus 40 (SV40) late polyadenylation site (SV4 LpA), a second ITR (ITR2), a prokaryotic origin of replication (ori), and a kanamycin (kan) selection gene, wherein the length between ITR1 and ITR2, including the payload (e.g., hPGK and hFXNco), is about 4.3 Kb in length.

FIGS. 3A and 3B are photographs and a graph, respectively, showing an anti-frataxin immunoflourescence expression-based potency assay. FIG. 3A is a set of photographs of cells derived from murine skeletal muscle (C2C12) stained for frataxin following their transduction with 1×10⁷ (1 e7) viral genome (vg)/cell of an AAV2/8 viral vector for expression of hFXNco. FIG. 3B is a graph depicting the frataxin immunofluorescence described in FIG. 3A as a function of the multiplicity of infection (MOI), as normalized to the intensity of a Hoechst counter-stain.

FIG. 4 is an immunoblot showing expression of intermediate and mature isoforms of frataxin in C2C12 cells transduced with an exemplary AAV2/8 viral vector for the expression of hFXNco, as described in FIGS. 1 and 2, in an amount of 1×10⁶ (1e6) vg/cell or 1×10⁷ (1e7) vg/cell, respectively.

FIG. 5 is a graph depicting the probability of survival of FXN knockout (KO) mice across time after the intravenous (i.v.) administration of an exemplary AAV2/8 viral vector for the expression of a hPGK promoter-driven hFXNco or murine FXN variant 1 (mFXN) in an amount of 3×10¹³ (3e13) vg/kg or 1×10¹⁴ (1e14) vg/kg, as compared to untransduced controls (Vehicle wild-type (WT) and Vehicle KO).

FIGS. 6A-E show the survival, body weight, and cardiac parameters of FXN KO mice after the i.v. administration of an exemplary AAV2/8 viral vector for the expression of a hPGK promoter-driven hFXNco (hFXN) or mFXN in an amount of 3×10¹³ vg/kg or 1×10¹⁴ vg/kg, as compared to untransduced controls (WT Vehicle and FRDA Vehicle). FIG. 6A is a graph depicting the probability of survival of FXN KO mice across time. Survival data are represented as the age at which the mice were euthanized if the mice displayed a greater than 20% body weight decrease, signs of respiratory distress, unresponsiveness to meaningful stimuli, and/or overall poor body condition prior to scheduled necropsy. FIG. 6B is a graph depicting male and female body weight of FXN KO mice across time. FIG. 6C is a graph showing heart weight of FXN KO mice by left ventricular mass normalized to body weight at the time of pre-treatment (six weeks of age), after treatment (9-10 weeks of age), and at lifespan (18-19 weeks of age). FIG. 6D is a graph showing ejection fraction of FXN KO mice at the time of pre-treatment (six weeks of age), after treatment (9-10 weeks of age), and at lifespan (18-19 weeks of age). FIG. 6E is a graph showing myosin light chain 3, a marker for myocardial damage, of FXN KO mice at the time of pre-treatment (six weeks of age), after treatment (9-10 weeks of age), and at lifespan (18-19 weeks of age).

FIGS. 7A and 7B are a set of graphs showing the frataxin expression in the heart in FXN KO mice after the i.v. administration of an exemplary AAV2/8 viral vector for the expression of hFXNco or mFXN in an amount of 3×10¹³ vg/kg or 1×10¹⁴ vg/kg, as compared to untransduced controls (Vehicle WT and Vehicle KO). FIG. 7A is a graph depicting dose-dependent frataxin expression in the heart as a function of vector copy number (VCN) per decigram (DG), whereas FIG. 7B depicts frataxin expression as the nanograms (ng) of protein per mg of biopsied heart tissue sampled.

FIG. 8 is a graph showing the level of frataxin protein in heart tissue, as measured by enzyme linked immunosorbent assay (ELISA), at 4 weeks (FIG. 8A) and 12 weeks (FIG. 8B) after administration of an exemplary AAV2/8 viral vector for the expression of hFXNco or mFXN in an amount of 3×10¹³ vg/kg or 1×10¹⁴ vg/kg, as compared to untransduced controls (WT Vehicle and FRDA Vehicle).

FIGS. 9A and 9B are graphs showing the number of vector copies per diploid genome detected in the heart tissue, as measured by qPCR, at 4 and 12 weeks after administration of an exemplary AAV2/8 viral vector for the expression of hFXNco or mFXN in an amount of 3×10¹³ vg/kg or 1×10¹⁴ vg/kg, as compared to untransduced controls (WT Vehicle and FRDA Vehicle).

FIG. 10A is immunohistochemistry of frataxin expression in heart tissue. FIG. 10B is a graph showing the percentage of cells exhibiting weak, moderate, or strong frataxin expression at four weeks after administration of an exemplary AAV2/8 viral vector for the expression of hFXNco or mFXN in an amount of 3×10¹³ vg/kg or 1×10¹⁴ vg/kg.

FIGS. 11A and 11B are photographs of cells stained for frataxin following their transduction with 1×10⁷ (1e7) viral genome (vg)/cell of Version 2 of an AAV2/8 viral vector for expression of hFXNco (AAV2/8-PGK-FXN V2) compared to untransduced controls (Vehicle control) and graphs depicting the frataxin immunofluorescence described as a function of the multiplicity of infection (MOI), as normalized to the intensity of a Hoechst counter-stain, for mouse skeletal muscle (FIG. 11A) and human skeletal muscle (FIG. 11B).

FIG. 12 is a comparison of AAV2/8-PGK-FXN Version 1 (V1) and Version 2 (V2). FIG. 12A is a collection of photographs of mouse skeletal muscle (C2C12) cells stained for frataxin following their transduction with 1×10⁷ (1 e7) viral genome (vg)/cell of AAV2/8-PGK-FXN V1 or V2 compared to untransduced controls (Vehicle control). FIG. 12B is a graph depicting the frataxin immunofluorescence described as a function of the multiplicity of infection (MOI), as normalized to the intensity of a Hoechst counter-stain, for AAV2/8-PGK-FXN V1 or V2. FIG. 12C is an immunoblot showing expression of mature isoforms of frataxin in C2C12 cells transduced with AAV2/8-PGK-FXN V1 or V2 in an amount of 1×10⁶ (1e6) vg/cell or 1×10⁷ (1e7) vg/cell.

FIG. 13 is a collection of graphs depicting the frataxin immunofluorescence described as a function of the multiplicity of infection (MOI), as normalized to the intensity of a Hoechst counter-stain, for Version 2 of an AAV2/8 viral vector for expression of FXN (AAV2/8-PGK-FXN V2), expressing either wild-type FXN (WT) or codon-optimized FXN (CO). FIG. 13A corresponds to transduced mouse skeletal muscle (C2C12) cells and FIG. 13B corresponds to transduced human cells.

FIG. 14 is a graph depicting survival of FXN KO mice across time after the intravenous administration of AAV2/8-PGK-FXN version 1 (V1) or version 2 (V2) at varying doses of viral genome (vg)/kilogram (kg), as compared to untransduced controls (Vehicle WT and Vehicle mutant). Survival data are represented as the age at which the mice were euthanized if the mice displayed any of the following conditions prior to scheduled necropsy: >20% decrease in body weight, demonstrating signs of respiratory distress, unresponsiveness to meaningful stimuli, and/or overall poor body condition.

FIG. 15 is a collection of graphs showing the ejection fraction the heart for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 5 weeks of age (WOA), 9-10 WOA, and 18-19 WOA.

FIG. 16 is a collection of graphs showing the percent of fractional shortening of the heart for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 5 weeks of age (WOA), 9-10 WOA, and 18-19 WOA.

FIG. 17 is a collection of graphs showing the left ventricular mass of the heart for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 5 weeks of age (WOA), 9-10 WOA, and 18-19 WOA.

FIG. 18 is a collection of graphs showing serum cardiac troponin for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 9-10 weeks of age (WOA) and 18-19 WOA.

FIG. 19 is a collection of graphs showing serum myosin light chain for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 9-10 weeks of age (WOA) and 18-19 WOA.

FIG. 20 is a collection of graphs showing serum aspartate transaminase (AST) for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 9-10 weeks of age (WOA) and 18-19 WOA.

FIG. 21 is a collection of graphs showing serum alanine transaminase (ALT) for FXN KO mice after the intravenous administration of AAV2/8-PGK-FXN V1 or V2 at varying doses of viral genome (vg)/kilogram (kg), as compared to untransduced controls (Vehicle WT and Vehicle mutant), at 9-10 weeks of age (WOA) and 18-19 WOA.

FIG. 22 is a collection of graphs showing vector copy number (VCN) per decigram (DG) of AAV2/8-PGK-FXN in the heart and quadricep of FXN KO mice at 4 weeks and 12 weeks after the intravenous administration of AAV2/8-PGK-FXN V1 or V2 at varying doses of viral genome (vg)/kilogram (kg).

FIG. 23 is a collection of graphs comparing the vector copy number (VCN) per decigram (DG) of AAV2/8-PGK-FXN in the heart and quadricep of FXN KO mice at 4 weeks and 12 weeks after the intravenous administration of AAV2/8-PGK-FXN V1 or V2 at varying doses of viral genome (vg)/kilogram (kg).

FIG. 24 is a collection of graphs showing vector copy number (VCN) per decigram (DG) of AAV2/8-PGK-FXN in liver of FXN KO mice at 4 weeks and 12 weeks after the intravenous administration of AAV2/8-PGK-FXN V1 or V2 at varying doses of viral genome (vg)/kilogram (kg).

FIG. 25 is a graph showing FXN mRNA transcript as RNA relative quantitation (RQ) in the heart and quadricep (Quad) of FXN KO mice after the intravenous administration of AAV2/8-PGK-FXN V1 or V2 at varying doses of viral genome (vg)/kilogram (kg).

FIG. 26 is a graph showing FXN protein expression in the heart of FXN KO mice at 4 weeks and 12 weeks after the intravenous administration of AAV2/8-PGK-FXN V1 or V2 at varying doses of viral genome (vg)/kilogram (kg), as compared to untransduced controls (WT vehicle and KO vehicle).

FIG. 27 is a graph showing FXN protein expression in the liver of FXN KO mice at 4 weeks and 12 weeks after the intravenous administration of AAV2/8-PGK-FXN V1 or V2 at varying doses of viral genome (vg)/kilogram (kg), as compared to untransduced controls (WT vehicle and KO vehicle).

FIG. 28 is a graph showing FXN protein expression in the quadricep of FXN KO mice at 4 weeks and 12 weeks after the intravenous administration of AAV2/8-PGK-FXN V1 or V2 at varying doses of viral genome (vg)/kilogram (kg), as compared to untransduced controls (WT vehicle and KO vehicle).

FIG. 29 is a graph depicting the time in seconds that it takes to fall off of a rotarod for FXN KO mice after the intracerebroventricular (ICV) administration of AAV8-PGK-FXN V1 or V2 at varying doses of viral genome (vg)/animal, as compared to untransduced controls (Vehicle control and Vehicle mutant). Measurements were taken at 6-8 weeks of age (WOA), 11-12 WOA, 13-14 WOA, and 16-18 WOA, with ICV administration at 8 WOA.

FIG. 30 is a collection of graphs showing vector copy number (VCN) per decigram (DG) of AAV8-PGK-FXN at the time of necropsy in the caudal spinal cord, cerebellum, cortex, rostral spinal cord, sciatic nerve, caudal dorsal root ganglion (DRG), half left liver lobe, heart half, and rostral DRG for FXN KO mice after the intracerebroventricular (ICV) administration of AAV8-PGK-FXN V2 at varying doses of viral genome (vg)/animal. ICV administration took place at 8 weeks old.

FIG. 31 is a collection of graphs showing vector copy number (VCN) per decigram (DG) of AAV8-PGK-FXN at the time of necropsy in the cerebellum, cortex, heart, and liver for FXN KO mice after the intracerebroventricular (ICV) or intraparenchymal (IPC) administration of AAV8-PGK-FXN V2 at varying doses of viral genome (vg)/animal. Animals were dosed at 8 weeks old. Tissues from ICV-dosed mice were analyzed at 10-11 weeks post dose and tissues from IPC-dosed mice were analyzed at 5 weeks post dose.

FIG. 32 is a collection of graphs showing FXN protein in the cortex and cerebellum at the time of necropsy for wild type (WT), untransduced (FXN^{PAV/null}), and FXN KO mice transduced via intracerebroventricular (ICV) or intraparenchymal (IPC) administration of AAV8-PGK-FXN V1 or V2 at varying doses of viral genome (vg)/animal. Animals were dosed at 8 weeks old. Tissues from ICV-dosed mice were analyzed at 10 weeks post dose and tissues from IPC-dosed mice were analyzed at 5 weeks post dose.

FIG. 33 is a graph showing FXN protein per vector copy number (VCN) in the cortex, cerebellum, heart, and liver for FXN KO mice after the intracerebroventricular (ICV) or intraparenchymal (IPC) administration of AAV8-PGK-FXN V2. Tissues were analyzed at 5 weeks post injection (wpi). ICV was administered on postnatal day 2 (PND2).

FIG. 34 is a graph showing neurofilament light chain (NFLC) in picograms (pg) per milliliter (mL) for FXN KO mice after the administration of AAV8-PGK-FXN V1 or V2 at varying doses of viral genome (vg)/animal, as compared to untransduced controls (WT Vehicle and KO Vehicle).

### DEFINITIONS

As used herein, the term “about” refers to a value that is within 10% above or below the value being described.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., Fields et al. Virology, 4^{th} ed. Lippincott-Raven Publishers, Philadelphia, 1996. Additional AAV serotypes and clades have been identified recently. (See, e.g., Gao et al. J. Virol. 78:6381 (2004); Moris et al. Virol. 33:375 (2004). The genomic sequences of various serotypes of AAV, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching AAV nucleic acid and amino acid sequences. See also, e.g., Bantel-Schaal et al. J. Virol. 73:939 (1999); Chiorini et al. J. Virol. 71:6823 (1997); Chiorini et al. J. Virol. 73:1309 (1999); Gao et al. Proc. Nat. Acad. Sci. USA 99:11854 (2002); Moris et al. Virol. 33:375 (2004); Muramatsu et al. Virol. 221:208 (1996); Ruffing et al. J. Gen. Virol. 75:3385 (1994); Rutledge et al. J. Virol. 72:309 (1998); Schmidt et al. J. Virol. 82:8911 (2008); Shade et al. J. Virol. 58:921 (1986); Srivastava et al. J. Virol. 45:555 (1983); Xiao et al. J. Virol. 73:3994 (1999); WO 00/28061, WO 99/61601, WO 98/11244; and U.S. Pat. No. 6,156,303; the disclosures of which are incorporated by reference herein for teaching AAV nucleic acid and amino acid sequences.

A “capsid protein” as used herein refers to any of the AAV capsid proteins that are components of AAV viral particles, including AAV8 and AAV9.

As used herein, the term “cloning site” refers to a nucleic acid sequence containing a restriction site for restriction endonuclease-mediated cloning by ligation of a nucleic acid containing compatible cohesive or blunt ends, a region of nucleic acid serving as a priming site for PCR-mediated cloning of insert DNA by homology and extension “overlap PCR stitching”, or a recombination site for recombinase-mediated insertion of target nucleic acids by recombination-exchange reaction, or mosaic ends for transposon mediated insertion of target nucleic acids, as well as other techniques common in the art.

The term “codon” as used herein refers to any group of three consecutive nucleotide bases in a given messenger RNA molecule, or coding strand of DNA, that specifies a particular amino acid or a starting or stopping signal for translation. The term codon also refers to base triplets in a DNA strand.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner known in the art, such as that described in, e.g., U.S. Pat. Nos. 7,561,972, 7,561,973, and 7,888,112, each of which is incorporated herein by reference in its entirety. For example, the sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated. As used herein, the terms “codon-optimized human frataxin gene” and “hFXNco” refer to a polynucleotide exhibiting at least 95% (e.g., 95%, 97%, 98%, or 99%) sequence identity to an endogenous RNA molecule encoding the frataxin protein variant 1 (e.g., SEQ ID NO: 5). In some embodiments, the hFXNco is identical to SEQ ID NO: 1.

Throughout this specification and claims, the word “comprise,” or variations such as “comprises” or “comprising,” will be understood to imply the inclusion of a stated integer or group of integers but not the exclusion of any other integer or group of integers.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

By “CpG sites” is meant regions of DNA where a cytosine nucleotide occurs next to a guanine nucleotide in the linear nucleic acid sequence of nucleotides along its length, e.g., -C-phosphate-G-cytosine and guanine separated by only one phosphate, or a cytosine 5′ to the guanine nucleotide.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

The term “frataxin” refers to the frataxin protein, and the term “FXN” refers to the gene (also referred to in the art as “FA,” “X25,” “CyaY” “FARR,” and “MGC57199”) encoding the frataxin protein. As used herein, the terms “frataxin” and “FXN” interchangeably refer to the polypeptide and nucleic acid, respectively, including polymorphic variants, alleles, mutants, and interspecies homologs that: (1) have an amino acid sequence that has greater than about 905 amino acid sequence identity, for example, 96%, 97%, 98% or 99% or greater amino acid sequence identity, preferably over a region of at least about 25, 50, 100, 200, 300, 400, or more amino acids, or over the full-length, to an amino acid sequence encoded by a FXN nucleic acid (SEQ ID NO: 5; see, e.g., GenBank Accession Nos. NM-000144.4 (isoform 1); or to an amino acid sequence of a frataxin polypeptide (SEQ ID NO: 3; see, e.g., GenBank Accession Nos. NP-000135.2 (isoform 1);); have a nucleic acid sequence that has greater than about 95%, for example greater than about 96%, 97%, 98%, 99%, or higher nucleotide sequence identity, preferably over a region of at least about 25, 50, 100, 200, 500, 1000, 2000 or more nucleotides, or over the full-length, to a FXN nucleic acid (e.g., frataxin polynucleotides, as described herein, and FXN polynucleotides that encode frataxin polypeptides, as described herein).

As used herein, the term “Friedreich ataxia” refers to an autosomal recessive congenital ataxia caused by a mutation in gene FXN (formerly known as X25) that encodes frataxin, located on chromosome 9. The genetic basis for Friedreich ataxia involves GAA trinucleotide repeats in an intron region of the gene encoding frataxin. This segment is normally repeated 5 to 33 times within the FXN gene. In people with Friedreich ataxia, the GAA segment is repeated 66 to more than 1,000 times. People with GAA segments repeated fewer than 300 times tend to have a later appearance of symptoms (after age 25) than those with larger GAA trinucleotide repeats. The presence of these repeats results in reduced transcription and expression of the gene. Frataxin is involved in regulation of mitochondrial iron content. The mutation in the FXN gene causes progressive damage to the nervous system, resulting in symptoms ranging from gait disturbance to speech problems; it can also lead to heart disease and diabetes. The ataxia of Friedreich ataxia results from the degeneration of nerve tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses). A subject with Friedreich ataxia may exhibit one or more of the following symptoms: muscle weakness in the arms and legs, loss of coordination, vision impairment, hearing impairment, slurred speech, curvature of the spine (scoliosis), high plantar arches (pes cavus deformity of the foot), carbohydrate intolerance, diabetes mellitus, heart disorders (e.g., atrial fibrillation, tachycardia (fast heart rate) and hypertrophic cardiomyopathy). A subject with Friedreich ataxia may further exhibit involuntary and/or rapid eye movements, loss of deep tendon reflexes, loss of extensor plantar responses, loss of vibratory and proprioceptive sensation, cardiomegaly, symmetrical hypertrophy, heart murmurs, and heart conduction defects. Pathological analysis may reveal sclerosis and degeneration of dorsal root ganglia, spinocerebellar tracts, lateral corticospinal tracts, and posterior columns.

As used herein, the term “GC content” refers to the quantity of nucleosides in a particular nucleic acid molecule, such as a DNA or RNA polynucleotide, that are either guanosine (G) or cytidine (C) relative to the total quantity of nucleosides present in the nucleic acid molecule. GC content may be expressed as a percentage, for instance, according to the following formula:

$$GC Content = ( ( Total quantity of guanosine nucleosides ) + ( Total quantity of cytidine nucleosides ) / ( Total quantity of nucleosides ) ) \times 100$$

As used herein, the term “gene” refers to a region of DNA that encodes a protein. A gene may include regulatory regions and a protein-coding region. In some embodiments, a gene includes two or more introns and three or more exons, wherein each intron forms an intervening sequence between two exons.

As used herein, the term “intron” refers to a region within the coding region of a gene, the nucleotide sequence of which is not translated into the amino acid sequence of the corresponding protein. The term intron also refers to the corresponding region of the RNA transcribed from a gene. In some embodiments, a gene, for example, may contain at minimum two introns, each of which forms the intervening sequence between two exons. Introns are transcribed into pre-mRNA, but are removed during processing, and are not included in the mature mRNA.

An “ITR” is a palindromic nucleic acid, e.g., an inverted terminal repeat, that is about 120 nucleotides to about 250 nucleotides in length and capable of forming a hairpin. The term “ITR” includes the site of the viral genome replication that can be recognized and bound by a parvoviral protein (e.g., Rep78/68). An ITR may be from any adeno-associated virus (AAV), with serotype 2 being preferred. An ITR includes a replication protein binding element (RBE) and a terminal resolution sequences (TRS). The term “ITR” does not require a wild-type parvoviral ITR (e.g., a wild-type nucleic acid sequence may be altered by insertion, deletion, truncation, or missense mutations), as long as the ITR functions to mediate virus packaging, replication, integration, and/or provirus rescue, and the like. The “5′ ITR” is intended to mean the parvoviral ITR located at the 5′ boundary of the nucleic acid molecule; and the term “3′ ITR” is intended to mean the parvoviral ITR located at the 3′ boundary of the nucleic acid molecule.

As used herein, the term “modified nucleotide” refers to a nucleotide or portion thereof (e.g., adenosine, guanosine, thymidine, cytidine, or uridine) that has been altered by one or more enzymatic or synthetic chemical transformations. Exemplary alterations observed in modified nucleotides described herein or known in the art include the introduction of chemical substituents, such as halo, thio, amino, azido, alkyl, acyl, or other functional groups at one or more positions (e.g., the 2′, 3, and/or 5′ position) of a 2-deoxyribonucleotide or a ribonucleotide.

As used herein, the term “mutation” refers to a change in the nucleotide sequence of a gene or a change in the polypeptide sequence of a protein. Mutations in a gene or protein may occur naturally as a result of, for example, errors in DNA replication, DNA repair, irradiation, and exposure to carcinogens or mutations may be induced as a result of administration of a transgene expressing a mutant gene. Mutations may result from single or multiple nucleotide insertions, deletions, or substitutions.

“Nucleic acid” and “polynucleotide,” as used interchangeably herein, refer to polymers of nucleotides of any length and include DNA and RNA.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., Fields et al. Virology, 4^{th} ed. Lippincott-Raven Publishers, Philadelphia, 1996. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for instance, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As one of skill in the art would understand, as used herein, for the purpose of determining “percent sequence identity”, a uridine nucleoside in an RNA molecule is considered equivalent to a thymidine nucleoside in a DNA molecule. Therefore, an RNA equivalent may be considered to have 100% sequence identity to a DNA polynucleotide if the RNA equivalent and DNA polynucleotide differ from one another only by the substitution of uridine nucleosides in the RNA equivalent with thymidine nucleosides in the DNA polynucleotide.

The term “polyadenylation signal” or “polyadenylation site” is used to herein to mean a nucleic acid sequence sufficient to direct the addition of polyadenosine ribonucleic acid to an RNA molecule expressed in a cell.

A “promoter” is a nucleic acid enabling the initiation of the transcription of a gene in a messenger RNA, such transcription being initiated with the binding of an RNA polymerase on or nearby the promoter.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject. As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “RNA equivalent” of a gene refers to a RNA polynucleotide that corresponds to a DNA polynucleotide that encodes the gene, such as a RNA transcript obtainable by transcription of a DNA polynucleotide that contains the gene. Exemplary RNA equivalents include mRNA transcripts produced synthetically, such as by way of solid phase nucleic acid synthesis techniques known in the art and/or described herein, as well as by recombinant nucleic acid preparation methods.

A “spacer” is any polynucleotide of at least 1.0 Kb in length that contains an open reading frame (ORF) of less than 100 amino acids; has a CpG content that is less than 1% of the total nucleic acid sequence; or does not contain transcription factor (TF) binding sites (e.g., sites recognized by a prokaryotic or baculoviral transcription factor). The term spacer does not include nucleic acids of prokaryotic or baculoviral origin. A spacer may be isolated from a naturally occurring source or modified, e.g., to reduce the size of an ORF, the CpG content, or number of transcription factor binding sites. A spacer may be selected from naturally occurring nucleic acids that promote expression of a polynucleotide, e.g., an intron found adjacent to an ORF or an enhancer found near a transcriptional start site. Use of a “spacer,” as defined herein, results in a reduction of contaminating nucleic acids packaged into a viral particle.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Goeddel, Gene Expression Technology: Methods in Enzymology 185 (Academic Press, San Diego, CA, 1990).

As used herein, the terms “treat” or “treatment” refer to therapeutic treatment, in which the object is to prevent or slow down (lessen) an undesired physiological change or disorder, such Friedreich ataxia, among others. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. In the context of Friedreich ataxia, treatment of a patient may manifest in one or more detectable changes, such as an increase in the concentration of frataxin or nucleic acids (e.g., DNA or RNA, such as mRNA) encoding frataxin (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 10-fold, 20-fold, 30-fold, 40-fold, 50-fold, 60-fold, 70-fold, 80-fold, 90-fold, 100-fold, 500-fold, 1,000-fold, or more). The concentration of frataxin may be determined using protein detection assays known in the art, including ELISA assays described herein. The concentration of frataxin-encoding nucleic acids may be determined using nucleic acid detection assays (e.g., RNA Seq assays) described herein. Exemplary protocols for the detection of frataxin proteins and nucleic acids are provided in Example 3, below. Additionally, treatment of a patient suffering from Friedreich ataxia may manifest in improvements in a patient's muscle function (e.g., cardiac or skeletal muscle function) as well as improvements in muscle coordination. For example, treatment of a patient suffering from Friedreich ataxia may manifest in a reduction in Total Friedreich Ataxia Rating Scale (FARS) Score (e.g., by about 12 weeks after treatment).

As used herein, the term “vector” refers to a nucleic acid, e.g., DNA or RNA, that may function as a vehicle for the delivery of a gene of interest into a cell (e.g., a mammalian cell, such as a human cell), such as for purposes of replication and/or expression. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### DETAILED DESCRIPTION

The compositions and methods described herein are useful for stimulating expression of the human frataxin protein and for treating disorders associated with mutations in the frataxin gene (FXN), such as Frederich ataxia. The compositions described herein include a plasmid (e.g., a viral vector e.g., an adeno-associated virus (AAV)) encoding a codon-optimized human FXN (hFXNco) or RNA equivalent thereof for expression of frataxin protein in a cell. The plasmids described herein include an AAV including two inverted terminal repeats (ITR; e.g., a first ITR (ITR1) and a second ITR (ITR2)), wherein the length of the nucleic acids between and including ITR1 and ITR2 is about 3.9 Kb to about 4.7 Kb. Without being limited by mechanism, the compositions described herein may ameliorate pathology associated with Frederich ataxia by efficaciously stimulating the expression of the human frataxin protein.

The present invention is based, at least in part, on the discovery that delivery of a nucleic acid molecule including ITR1-hFXNco-ITR2, wherein the length of the nucleic acids between and including ITR1 and ITR2 is about 3.9 Kb to about 4.7 Kb, leads to a surprisingly superior ability to induce the expression of human frataxin protein in a cell. This property is particularly beneficial in view of the prevalence of mutations of the FXN gene in mammalian genomes, such as in the genomes of human patients with Frederich ataxia. Using the compositions and methods described herein, the expression of important, healthy FXN or RNA transcripts thereof and their encoded frataxin protein product can be efficaciously enhanced.

The sections that follow provide a description of exemplary codon-optimization and methods of production thereof to produce hFXNco that may be used in conjunction with the vectors encoding such constructs described herein and methods that may be used to treat Frederich ataxia.

### Therapeutic Proteins

Genes that can be incorporated into a plasmid (e.g., a viral vector) according to the methods described herein include those that encode therapeutic proteins (e.g., frataxin), such as those that can be transferred to a subject (e.g., a human patient) suffering from a disease or condition (e.g., Frederich ataxia) characterized by a deficiency in the protein. For instance, a gene that can be delivered to a patient according to the methods described herein include a gene encoding frataxin.

In one approach, the invention provides a human FXN (hFXN) or RNA equivalent thereof having a polynucleotide sequence that is at least 95% (e.g., 95%, 96%, 97%, 98%, or 99%) identical to an endogenous RNA molecule encoding the frataxin protein variant 1 (e.g., SEQ ID NO: 5). For example, in some embodiments, the polynucleotide exhibits at least 95% sequence identity to an endogenous RNA molecule encoding the frataxin protein variant 1 (e.g., SEQ ID NO: 5). In some embodiments, the polynucleotide exhibits at least 96% sequence identity to an endogenous RNA molecule encoding the frataxin protein variant 1 (e.g., SEQ ID NO: 5). In some embodiments, the polynucleotide exhibits at least 97% sequence identity to an endogenous RNA molecule encoding the frataxin protein variant 1 (e.g., SEQ ID NO: 5). In some embodiments, the polynucleotide exhibits at least 98% sequence identity to an endogenous RNA molecule encoding the frataxin protein variant 1 (e.g., SEQ ID NO: 5). In some embodiments, the polynucleotide exhibits at least 99% sequence identity to an endogenous RNA molecule encoding the frataxin protein variant 1 (e.g., SEQ ID NO: 5). In some embodiments, the polynucleotide is identical to an endogenous RNA molecule encoding the frataxin protein variant 1 (e.g., SEQ ID NO: 5).

In some embodiments, the hFXN, or an RNA equivalent thereof, encodes a protein that is at least 85% (e.g., 85%, 86%, 87%, 88%, 89%, 90%, 91%, 92%, 93%, 94%, 95%, 96%, 97%, 98%, or 99%) identical to the amino acid sequence of SEQ ID NO: 3. For example, in some embodiments, the hFXN, or an RNA equivalent thereof, encodes a protein that is at least 86% identical to the amino acid sequence of SEQ ID NO: 3. In some embodiments, the hFXN, or an RNA equivalent thereof, encodes a protein that is at least 87% identical to the amino acid sequence of SEQ ID NO: 3. In some embodiments, the hFXN, or an RNA equivalent thereof, encodes a protein that is at least 88% identical to the amino acid sequence of SEQ ID NO: 3. In some embodiments, the hFXN, or an RNA equivalent thereof, encodes a protein that is at least 89% identical to the amino acid sequence of SEQ ID NO: 3. In some embodiments, the hFXN, or an RNA equivalent thereof, encodes a protein that is at least 90% identical to the amino acid sequence of SEQ ID NO: 3. In some embodiments, the hFXN, or an RNA equivalent thereof, encodes a protein that is at least 91% identical to the amino acid sequence of SEQ ID NO: 3. In some embodiments, the hFXN, or an RNA equivalent thereof, encodes a protein that is at least 92% identical to the amino acid sequence of SEQ ID NO: 3. In some embodiments, the hFXN, or an RNA equivalent thereof, encodes a protein that is at least 93% identical to the amino acid sequence of SEQ ID NO: 3. In some embodiments, the hFXN, or an RNA equivalent thereof, encodes a protein that is at least 94% identical to the amino acid sequence of SEQ ID NO: 3. In some embodiments, the hFXN, or an RNA equivalent thereof, encodes a protein that is at least 95% identical to the amino acid sequence of SEQ ID NO: 3. In some embodiments, the hFXN, or an RNA equivalent thereof, encodes a protein that is at least 96% identical to the amino acid sequence of SEQ ID NO: 3. In some embodiments, the hFXN, or an RNA equivalent thereof, encodes a protein that is at least 97% identical to the amino acid sequence of SEQ ID NO: 3. In some embodiments, the hFXN, or an RNA equivalent thereof, encodes a protein that is at least 98% identical to the amino acid sequence of SEQ ID NO: 3. In some embodiments, the hFXN, or an RNA equivalent thereof, encodes a protein that is at least 99% identical to the amino acid sequence of SEQ ID NO: 3. In some embodiments, the hFXN, or an RNA equivalent thereof, encodes a protein that is identical to the amino acid sequence of SEQ ID NO: 3.

### Codon Optimization

The compositions and methods described herein can be used to optimize the nucleic acid sequence of a gene or RNA equivalent thereof (e.g., FXI) encoding a protein of interest (e.g., frataxin) so as to achieve, for instance, enhanced expression of the protein in a particular cell type. For example, using the compositions and methods described herein, genes and RNA equivalents thereof can be optimized for tissue-specific expression of an encoded protein (e.g., frataxin). Genes and RNA equivalents thereof optimized using the compositions and methods described herein can be synthesized by chemical synthesis techniques and may be amplified, for instance, using polymerase chain reaction (PCR)-based amplification methods or by transfection of the gene into a cell, such as a bacterial cell or mammalian cell capable of replicating exogenous nucleic acids.

The genes and RNA equivalents described herein can have important clinical utility. A variety of diseases and conditions, including heritable genetic disorders, such as Frederich ataxia, are manifestations of a deficiency in a native protein (e.g., frataxin). With the advent of gene therapy, a wide array of vectors and gene delivery techniques have been developed for the introduction of exogenous protein-coding nucleic acids into target cells (e.g., human cells). However, there remains a need for optimized variants of the transgene encoded by the exogenous nucleic acid so as to achieve robust and stable expression of the encoded protein in the cell of interest.

### Reducing CpG Content and Homopolymer Content

Codon optimization may be performed by techniques known in the art. As a non-limiting example, one of skill in the art can manipulate the protein-encoding gene sequence of a target gene by incorporating codon substitutions that diminish the CpG content and/or homopolymer content of the gene. For instance, one can begin with a wild-type gene sequence and introduce substitutions (e.g., single-nucleotide substitutions) that reduce the CpG content and/or homopolymer content of the gene while preserving the identity of the encoded proteins sequence. One can then follow the sequence identity minimization process described above and in Example 1 in order to obtain a gene sequence that minimally resembles the gene (e.g., FXN) encoded in a cell type of interest. Alternatively, one can begin with a sequence that has been codon-optimized according to the sequence identity minimization process described above and subsequently can be manipulated by the introduction of mutations (e.g., single-nucleotide substitutions) that reduce the CpG content and/or homopolymer content of the gene. CpG sites and homopolymers can promote +1 frameshifts during the mRNA translation process. Alternatively, if the homopolymer encodes amino acid residues that are not essential for protein function (for instance, if the encoded amino acids are not present within the active site of an encoded enzyme or within a site necessary for non-covalent binding to another biological molecule), one of skill in the art can incorporate codon substitutions that interrupt the homopolymer and that introduce a conservative substitution into the encoded protein at the site of the corresponding amino acid.

### Preparation of Codon-Optimized Genes

Once designed, the final codon-optimized gene can be prepared, for instance, by solid phase nucleic acid procedures known in the art. For instance, to perform the chemical synthesis of nucleic acid molecules, such as DNA, RNA and the like, a solid phase synthesis process using a phosphoramidite method can be employed. According to this procedure, a nucleic acid is generally synthesized by the following steps.

First, a 5-OH-protected nucleoside that will occur at the 3′ terminal end of the nucleic acid to be synthesized is esterified via the 3′-OH function to a solid support by appending the nucleoside to a cleavable linker. Then, the support for solid phase synthesis on which the nucleoside is immobilized can be placed in a reaction column which is then set on an automated nucleic acid synthesizer.

Thereafter, an iterative synthetic process including the following steps can be performed in the reaction column according to a synthesis program of the automated nucleic acid synthesizer:
(1) a step of deprotection of the 5′-OH moiety of the protected, immobilized nucleoside (e.g., with an acid such as trichloroacetic acid in dichloromethane solution or the like to remove acid-labile hydroxyl protecting groups);
(2) a step of coupling a 5-OH-protected nucleosidephosphoramidite with the deprotected 5′—OH group of the immobilized nucleoside in the presence of an activator (e.g., tetrazole or the like);
(3) a step of capping the unreacted 5′—OH group of the 3′-terminal nucleoside (e.g., with acetic anhydride or the like); and
(4) a step of oxidizing the immobilized phosphite substituent (e.g., with aqueous iodine or the like).

The above process can be repeated to elongate the nucleic acid as needed in a 3′-to-5′ direction. 5′ terminal direction is promoted, and a nucleic acid having a desired sequence is synthesized.

Lastly, the cleavable linker is hydrolyzed (e.g., with aqueous ammonia, methylamine solution, or the like) to cleave the synthesized nucleic acid from the solid phase support. Procedures such as the foregoing for the chemical synthesis of nucleic acids are known in the art and are described, for instance, in U.S. Pat. No. 8,835,656, the disclosure of which is incorporated herein by reference as it pertains to protocols for the synthesis of nucleic acid molecules.

Additionally, the prepared gene can be amplified, for instance, using PCR-based techniques described herein or known in the art, and/or by transformation of DH5α E. coli with a plasmid containing the designed gene. The bacteria can subsequently be cultured so as to amplify the DNA therein, and the gene can be isolated plasmid purification techniques known in the art, followed optionally by a restriction digest and/or sequencing of the plasmid to verify the identity codon-optimized gene.

### Exemplary Codon-Optimized Human FXN

In one approach, the invention provides a hFXNco or RNA equivalent thereof having a polynucleotide sequence that is at least 85% (e.g., 85%, 86%, 87%, 88%, 89%, 90%, 91%, 92%, 93%, 94%, 95%, 96%, 97%, 98%, co or 99%) identical to the nucleic acid sequence of SEQ ID NO: 1. For example, in some embodiments, the hFXNco, or an RNA equivalent thereof, has a nucleic acid sequence that is at least 86% identical to the nucleic acid sequence of SEQ ID NO: 1. In some embodiments, the hFXNco, or an RNA equivalent thereof, has a nucleic acid sequence that is at least 87% identical to the nucleic acid sequence of SEQ ID NO: 1. In some embodiments, the hFXNco, or an RNA equivalent thereof, has a nucleic acid sequence that is at least 88% identical to the nucleic acid sequence of SEQ ID NO: 1. In some embodiments, the hFXNco, or an RNA equivalent thereof, has a nucleic acid sequence that is at least 89% identical to the nucleic acid sequence of SEQ ID NO: 1. In some embodiments, the hFXNco, or an RNA equivalent thereof, has a nucleic acid sequence that is at least 90% identical to the nucleic acid sequence of SEQ ID NO: 1. In some embodiments, the hFXNco, or an RNA equivalent thereof, has a nucleic acid sequence that is at least 91% identical to the nucleic acid sequence of SEQ ID NO: 1. In some embodiments, the hFXNco, or an RNA equivalent thereof, has a nucleic acid sequence that is at least 92% identical to the nucleic acid sequence of SEQ ID NO: 1. In some embodiments, the hFXNco, or an RNA equivalent thereof, has a nucleic acid sequence that is at least 93% identical to the nucleic acid sequence of SEQ ID NO: 1. In some embodiments, the hFXNco, or an RNA equivalent thereof, has a nucleic acid sequence that is at least 94% identical to the nucleic acid sequence of SEQ ID NO: 1. In some embodiments, the hFXNco, or an RNA equivalent thereof, has a nucleic acid sequence that is at least 95% identical to the nucleic acid sequence of SEQ ID NO: 1. In some embodiments, the hFXNco, or an RNA equivalent thereof, has a nucleic acid sequence that is at least 96% identical to the nucleic acid sequence of SEQ ID NO: 1. In some embodiments, the hFXNco, or an RNA equivalent thereof, has a nucleic acid sequence that is at least 97% identical to the nucleic acid sequence of SEQ ID NO: 1. In some embodiments, the hFXNco, or an RNA equivalent thereof, has a nucleic acid sequence that is at least 98% identical to the nucleic acid sequence of SEQ ID NO: 1. In some embodiments, the hFXNco, or an RNA equivalent thereof, has a nucleic acid sequence that is at least 99% identical to the nucleic acid sequence of SEQ ID NO: 1.

In some embodiments, the hFXNco, or an RNA equivalent thereof, has the nucleic acid sequence of SEQ ID NO: 1.

### Methods for the Delivery of Exogenous Nucleic Acids to Target Cells

### Transfection Techniques

Techniques that can be used to introduce a transgene, such as a transgene operably linked to a transcription regulatory element described herein, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Res 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Exp. Dermatol. 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., JoVE 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Top Curr Chem 228:227 (2003), the disclosure of which is incorporated herein by reference) and diethylaminoethyl (DEAE)-dextran, the use of which as a transfection agent is described in detail, for example, in Gulick et al., Curr Protoc Mol Biol 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods Cell Biol. 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats (ITR). Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nat. Biotechnol. 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nat. Rev. Genet. 11:636 (2010); and in Joung et al., Nat. Rev. Mol. Cell Biol. 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Vectors for Delivery of Exogenous Nucleic Acids to Target Cells

### Viral Vectors for Nucleic Acid Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the invention include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Nucleic Acid Delivery

In some embodiments, nucleic acids of the compositions and methods described herein are incorporated into a recombinant AAV (rAAV) vectors and/or virions in order to facilitate their introduction into a cell. rAAV vectors useful in the invention are recombinant nucleic acid constructs that include (1) a transgene to be expressed (e.g., a polynucleotide encoding a frataxin protein) and (2) viral nucleic acids that facilitate integration and expression of the heterologous genes. The viral nucleic acids may include those sequences of AAV that are required in cis for replication and packaging (e.g., functional ITRs) of the DNA into a virion. In typical applications, the transgene encodes frataxin, which is useful for correcting a frataxin-deficiency in patients suffering from Frederich ataxia. Such rAAV vectors may also contain marker or reporter genes. Useful rAAV vectors have one or more of the AAV WT genes deleted in whole or in part, but retain functional flanking ITR sequences. The AAV ITRs may be of any serotype (e.g., derived from serotype 2) suitable for a particular application. In some embodiments, the AAV ITRs may be AAV serotype 2 ITRs. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. In some embodiments, the AAV of the invention comprise a recombinant capsid protein. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of FXN in muscle cells. rAAV serotype 9 has also been found to be an efficient transducer of muscle cells. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 or AAV9 vector encoding a therapeutic protein (e.g., frataxin) pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Exemplary AAV Vectors

As described herein, exemplary AAV vector components may include a promoter, an intron, a polynucleotide encoding human FXN or a polynucleotide encoding hFXNco, and/or a polyadenylation site (pA).

In some embodiments, the AAV may include a prokaryotic promoter (P_{Euk}). In some embodiments, the AAV may include a muscle specific promoter. In some embodiments, the muscle specific promoter is a phosphoglycerate kinase (PGK) promoter, a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3, a cytomegalovirus promoter, or a chicken-β-actin promoter. For example, in some embodiments, the muscle specific promoter is a PGK promoter.

In some embodiments, the PGK promoter has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 2. For example, in some embodiments, the PGK promoter has a nucleic acid sequence that is at least 86% identical to the nucleic acid sequence of SEQ ID NO: 2. In some embodiments, the PGK promoter has a nucleic acid sequence that is at least 87% identical to the nucleic acid sequence of SEQ ID NO: 2. In some embodiments, the PGK promoter has a nucleic acid sequence that is at least 88% identical to the nucleic acid sequence of SEQ ID NO: 2. In some embodiments, the PGK promoter has a nucleic acid sequence that is at least 89% identical to the nucleic acid sequence of SEQ ID NO: 2. In some embodiments, the PGK promoter has a nucleic acid sequence that is at least 90% identical to the nucleic acid sequence of SEQ ID NO: 2. In some embodiments, the PGK promoter has a nucleic acid sequence that is at least 91% identical to the nucleic acid sequence of SEQ ID NO: 2. In some embodiments, the PGK promoter has a nucleic acid sequence that is at least 92% identical to the nucleic acid sequence of SEQ ID NO: 2. In some embodiments, the PGK promoter has a nucleic acid sequence that is at least 93% identical to the nucleic acid sequence of SEQ ID NO: 2. In some embodiments, the PGK promoter has a nucleic acid sequence that is at least 94% identical to the nucleic acid sequence of SEQ ID NO: 2. In some embodiments, the PGK promoter has a nucleic acid sequence that is at least 95% identical to the nucleic acid sequence of SEQ ID NO: 2. In some embodiments, the PGK promoter has a nucleic acid sequence that is at least 96% identical to the nucleic acid sequence of SEQ ID NO: 2. In some embodiments, the PGK promoter has a nucleic acid sequence that is at least 97% identical to the nucleic acid sequence of SEQ ID NO: 2. In some embodiments, the PGK promoter has a nucleic acid sequence that is at least 98% identical to the nucleic acid sequence of SEQ ID NO: 2. In some embodiments, the PGK promoter has a nucleic acid sequence that is at least 99% identical to the nucleic acid sequence of SEQ ID NO: 2. In some embodiments, the PGK promoter has a nucleic acid sequence that is identical to the nucleic acid sequence of SEQ ID NO: 2.

In some embodiments, the AAV includes an intron. In some embodiments, the intron is an SV40 intron.

In some embodiments, the intron is positioned 5′ to the polynucleotide.

In some embodiments, the hFXN or RNA equivalent thereof has a polynucleotide sequence that is at least 95% (e.g., 95%, 96%, 97%, 98%, or 99%) identical to an endogenous RNA molecule encoding the frataxin protein variant 1 (e.g., SEQ ID NO: 5). For example, in some embodiments, the polynucleotide exhibits at least 95% sequence identity to an endogenous RNA molecule encoding the frataxin protein variant 1 (e.g., SEQ ID NO: 5). In some embodiments, the polynucleotide exhibits at least 96% sequence identity to an endogenous RNA molecule encoding the frataxin protein variant 1 (e.g., SEQ ID NO: 5). In some embodiments, the polynucleotide exhibits at least 97% sequence identity to an endogenous RNA molecule encoding the frataxin protein variant 1 (e.g., SEQ ID NO: 5). In some embodiments, the polynucleotide exhibits at least 98% sequence identity to an endogenous RNA molecule encoding the frataxin protein variant 1 (e.g., SEQ ID NO: 5). In some embodiments, the polynucleotide exhibits at least 99% sequence identity to an endogenous RNA molecule encoding the frataxin protein variant 1 (e.g., SEQ ID NO: 5). In some embodiments, the polynucleotide is identical to an endogenous RNA molecule encoding the frataxin protein variant 1 (e.g., SEQ ID NO: 5).

In some embodiments, the hFXN, or an RNA equivalent thereof, encodes a protein that is at least 85% (e.g., 85%, 86%, 87%, 88%, 89%, 90%, 91%, 92%, 93%, 94%, 95%, 96%, 97%, 98%, or 99%) identical to the amino acid sequence of SEQ ID NO: 3. For example, in some embodiments, the hFXN, or an RNA equivalent thereof, encodes a protein that is at least 86% identical to the amino acid sequence of SEQ ID NO: 3. In some embodiments, the hFXN, or an RNA equivalent thereof, encodes a protein that is at least 87% identical to the amino acid sequence of SEQ ID NO: 3. In some embodiments, the hFXN, or an RNA equivalent thereof, encodes a protein that is at least 88% identical to the amino acid sequence of SEQ ID NO: 3. In some embodiments, the hFXN, or an RNA equivalent thereof, encodes a protein that is at least 89% identical to the amino acid sequence of SEQ ID NO: 3. In some embodiments, the hFXN, or an RNA equivalent thereof, encodes a protein that is at least 90% identical to the amino acid sequence of SEQ ID NO: 3. In some embodiments, the hFXN, or an RNA equivalent thereof, encodes a protein that is at least 91% identical to the amino acid sequence of SEQ ID NO: 3. In some embodiments, the hFXN, or an RNA equivalent thereof, encodes a protein that is at least 92% identical to the amino acid sequence of SEQ ID NO: 3. In some embodiments, the hFXN, or an RNA equivalent thereof, encodes a protein that is at least 93% identical to the amino acid sequence of SEQ ID NO: 3. In some embodiments, the hFXN, or an RNA equivalent thereof, encodes a protein that is at least 94% identical to the amino acid sequence of SEQ ID NO: 3. In some embodiments, the hFXN, or an RNA equivalent thereof, encodes a protein that is at least 95% identical to the amino acid sequence of SEQ ID NO: 3. In some embodiments, the hFXN, or an RNA equivalent thereof, encodes a protein that is at least 96% identical to the amino acid sequence of SEQ ID NO: 3. In some embodiments, the hFXN, or an RNA equivalent thereof, encodes a protein that is at least 97% identical to the amino acid sequence of SEQ ID NO: 3. In some embodiments, the hFXN, or an RNA equivalent thereof, encodes a protein that is at least 98% identical to the amino acid sequence of SEQ ID NO: 3. In some embodiments, the hFXN, or an RNA equivalent thereof, encodes a protein that is at least 99% identical to the amino acid sequence of SEQ ID NO: 3. In some embodiments, the hFXN, or an RNA equivalent thereof, encodes a protein that is identical to the amino acid sequence of SEQ ID NO: 3.

In some embodiments, the hFXNco or RNA equivalent thereof has a polynucleotide sequence that is at least 85% (e.g., 85%, 86%, 87%, 88%, 89%, 90%, 91%, 92%, 93%, 94%, 95%, 96%, 97%, 98%, co or 99%) identical to the nucleic acid sequence of SEQ ID NO: 1. For example, in some embodiments, the hFXNco, or an RNA equivalent thereof, has a nucleic acid sequence that is at least 86% identical to the nucleic acid sequence of SEQ ID NO: 1. In some embodiments, the hFXNco, or an RNA equivalent thereof, has a nucleic acid sequence that is at least 87% identical to the nucleic acid sequence of SEQ ID NO: 1. In some embodiments, the hFXNco, or an RNA equivalent thereof, has a nucleic acid sequence that is at least 88% identical to the nucleic acid sequence of SEQ ID NO: 1. In some embodiments, the hFXNco, or an RNA equivalent thereof, has a nucleic acid sequence that is at least 89% identical to the nucleic acid sequence of SEQ ID NO: 1. In some embodiments, the hFXNco, or an RNA equivalent thereof, has a nucleic acid sequence that is at least 90% identical to the nucleic acid sequence of SEQ ID NO: 1. In some embodiments, the hFXNco, or an RNA equivalent thereof, has a nucleic acid sequence that is at least 91% identical to the nucleic acid sequence of SEQ ID NO: 1. In some embodiments, the hFXNco, or an RNA equivalent thereof, has a nucleic acid sequence that is at least 92% identical to the nucleic acid sequence of SEQ ID NO: 1. In some embodiments, the hFXNco, or an RNA equivalent thereof, has a nucleic acid sequence that is at least 93% identical to the nucleic acid sequence of SEQ ID NO: 1. In some embodiments, the hFXNco, or an RNA equivalent thereof, has a nucleic acid sequence that is at least 94% identical to the nucleic acid sequence of SEQ ID NO: 1. In some embodiments, the hFXNco, or an RNA equivalent thereof, has a nucleic acid sequence that is at least 95% identical to the nucleic acid sequence of SEQ ID NO: 1. In some embodiments, the hFXNco, or an RNA equivalent thereof, has a nucleic acid sequence that is at least 96% identical to the nucleic acid sequence of SEQ ID NO: 1. In some embodiments, the hFXNco, or an RNA equivalent thereof, has a nucleic acid sequence that is at least 97% identical to the nucleic acid sequence of SEQ ID NO: 1. In some embodiments, the hFXNco, or an RNA equivalent thereof, has a nucleic acid sequence that is at least 98% identical to the nucleic acid sequence of SEQ ID NO: 1. In some embodiments, the hFXNco, or an RNA equivalent thereof, has a nucleic acid sequence that is at least 99% identical to the nucleic acid sequence of SEQ ID NO: 1.

In some embodiments, the polynucleotide has a nucleic acid sequence that is identical to the nucleic acid sequence of SEQ ID NO: 1.

In some embodiments, the AAV includes a polyadenylation site (pA). For example, the pA site may be selected from the non-limiting list comprising a SV40 late polyadenylation site, a SV40 early polyadenylation site, a human β-globin polyadenylation site, or a bovine growth hormone polyadenylation site. In some embodiments, the AAV includes a SV40 late polyadenylation site.

In some embodiments, the pA is positioned 3′ to the polynucleotide.

As described herein, exemplary AAV vector components, including a human phosphoglycerate kinase (hPGK) promoter, a hFXNco, as descried herein, are exemplified by the nucleic acid sequences in Table 2, shown below, a simian virus 40 (SV40) intron, and a SV40 late polyadenylation site.

TABLE 2
Exemplary AAV Vector Components Nucleic Acid Sequences

| | | Genetic |
|---|---|---|
| SEQ ID NO: | Nucleic Acid Sequence | Component |
| 2 | GGGGTTGGGGTTGCGCCTTTTCCAAGGCAGCCCTGGGTTTGCGC | Human PGK |
| | AGGGACGCGGCTGCTCTGGGCGTGGTTCCGGGAAACGCAGCGG | promoter |
| | CGCCGACCCTGGGTCTCGCACATTCTTCACGTCCGTTCGCAGCG | |
| | TCACCCGGATCTTCGCCGCTACCCTTGTGGGCCCCCCGGCGAC | |
| | GCTTCCTGCTCCGCCCCTAAGTCGGGAAGGTTCCTTGCGGTTCG | |
| | CGGCGTGCCGGACGTGACAAACGGAAGCCGCACGTCTCACTAG | |
| | TACCCTCGCAGACGGACAGCGCCAGGGAGCAATGGCAGCGCGC | |
| | CGACCGCGATGGGCTGTGGCCAATAGCGGCTGCTCAGCAGGGC | |
| | GCGCCGAGAGCAGCGGCCGGGAAGGGGCGGTGCGGGAGGCGG | |
| | GGTGTGGGGCGGTAGTGTGGGCCCTGTTCCTGCCCGCGCGGTG | |
| | TTCCGCATTCTGCAAGCCTCCGGAGCGCACGTCGGCAGTCGGCT | |
| | CCCTCGTTGACCGAATCACCGACCTCTCTCCCCAG | |
| 1 | ATGTGGACCCTGGGCAGGAGGGCTGTGGCTGGCCTGCTGGCCA | hFXNco |
| | GCCCCAGCCCTGCCCAGGCCCAGACCCTGACCAGGGTGCCCAG | |
| | GCCTGCTGAGCTGGCCCCCCTGTGTGGCAGGAGGGGCCTGAGG | |
| | ACTGACATTGATGCCACCTGCACCCCCAGGAGGGCCAGCAGCAA | |
| | CCAGAGGGGCCTGAACCAGATCTGGAATGTGAAGAAGCAGTCTG | |
| | TGTACCTGATGAACCTGAGGAAGTCTGGCACCCTGGGCCACCCT | |
| | GGCAGCCTGGATGAAACCACCTATGAGAGGCTGGCTGAGGAAAC | |
| | CCTGGACAGCCTGGCTGAGTTCTTTGAGGACCTGGCTGACAAGC | |
| | CCTACACCTTTGAGGACTATGATGTGAGCTTTGGCTCTGGGGTG | |
| | CTGACTGTGAAGCTGGGGGGGGACCTGGGCACCTATGTGATCAA | |
| | CAAGCAGACCCCCAACAAGCAGATCTGGCTGAGCAGCCCCAGCT | |
| | CTGGCCCCAAGAGATATGACTGGACTGGCAAGAACTGGGTGTAC | |
| | AGCCATGATGGGGTGAGCCTGCATGAGCTGCTGGCTGCTGAGCT | |
| | GACCAAGGCCCTGAAGACCAAGCTGGACCTGAGCAGCCTGGCC | |
| | TACTCTGGCAAGGATGCCTGA | |

In some embodiments, the AAV has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 4. For example, in some embodiments, the AAV has a nucleic acid sequence that is at least 86% identical to the nucleic acid sequence of SEQ ID NO: 4. In some embodiments, the AAV has a nucleic acid sequence that is at least 87% identical to the nucleic acid sequence of SEQ ID NO: 4. In some embodiments, the AAV has a nucleic acid sequence that is at least 88% identical to the nucleic acid sequence of SEQ ID NO: 4. In some embodiments, the AAV has a nucleic acid sequence that is at least 89% identical to the nucleic acid sequence of SEQ ID NO: 4. In some embodiments, the AAV has a nucleic acid sequence that is at least 90% identical to the nucleic acid sequence of SEQ ID NO: 4. In some embodiments, the AAV has a nucleic acid sequence that is at least 91% identical to the nucleic acid sequence of SEQ ID NO: 4. In some embodiments, the AAV has a nucleic acid sequence that is at least 92% identical to the nucleic acid sequence of SEQ ID NO: 4. In some embodiments, the AAV has a nucleic acid sequence that is at least 93% identical to the nucleic acid sequence of SEQ ID NO: 4. In some embodiments, the AAV has a nucleic acid sequence that is at least 94% identical to the nucleic acid sequence of SEQ ID NO: 4. In some embodiments, the AAV has a nucleic acid sequence that is at least 95% identical to the nucleic acid sequence of SEQ ID NO: 4. In some embodiments, the AAV has a nucleic acid sequence that is at least 96% identical to the nucleic acid sequence of SEQ ID NO: 4. In some embodiments, the AAV has a nucleic acid sequence that is at least 97% identical to the nucleic acid sequence of SEQ ID NO: 4. In some embodiments, the AAV has a nucleic acid sequence that is at least 98% identical to the nucleic acid sequence of SEQ ID NO: 4. In some embodiments, the AAV has a nucleic acid sequence that is at least 99% identical to the nucleic acid sequence of SEQ ID NO: 4. In some embodiments, the AAV has a nucleic acid sequence that is identical to the nucleic acid sequence of SEQ ID NO: 4.

As described herein, an exemplary AAV2/8 vector having the nucleic acid sequence of SEQ ID NO: 4, is shown below:

TCGCGCGTTTCGGTGATGACGGTGAAAACCTCTGACACATGCAGCTCCCGGACGTCATTGT
CGATCCTGCAGGCGTACGCCATCCAATGGAAAAGGGGGGGTTGGATTTCGCTTGTTGCATA
GGTTGGTCTCAAACTCCTGGCCTCAAGTGATTCTCCTGCCTCTGCCTCCCAAAGTGCTGAGA
TTACAGGTGTGAGGCACCATGCCAGGTCTCTTACTGTTTGTAATTAAATACATACACATTTTG
TGTGTTTGTGTGCACCTTTATAAAGTCAAAGGTGATAGTAACCCATTTAAGTTCCTACTCAATT
TTACTTTCCAGGGATAACTAACTACTTTTTCTTTTTGAGATGGAGTCTCGCTGTGTAGCCCAG
GCTGGAGTGCAGTGGCACCATCTCGGCTCACTGCAAGCTCCTCCTCCCTGGTTCACGCTAT
TCTCCTGCCTCAGCCTCCCCAACAACTAGGACTACAGGCTCACCTCGCCATACCTGGCTAAT
TTTTTGTATTTTTAGTAGAGACAGGGTTTCACTGTGTTAGCCAGGATGGTCTCGATCTCCTGA
CCTTGTGATCCGCCTGCCTCTGCCTCCCAAAGTGCTGGGATTACAGGCATGAGCAACCTCA
CCCAGCTGGGATAACTACTTTTTACAGGTTGATATTCTTTTGGACTTTTCCCCTGTGTAAAAA
TATACTATATTTGTTATGTACATATTATGTACATACAGACACAAATTGGACCATTCTCAGTATA
ATGATTCTCAGGTTTTTTTTTTTTTTTTTGAGGTGGGGAACTAGATAATTATGGACATCTTTCC
ATACTAGCATATCAATATCTACCTCATTCTTTTTAATATTTTTGCTAGTATTCCATTGTATGAAT
GTCCTATGATTTACTTAACCTGTCCATCAATATTTGTTTCCAGGTTTTTGCTATTATAATGCTG
CTGCAAAGTACATCCTCACACATCTTTATTTTGTCTATTCATATTTCTGTAAGATAGGTTACTA
AAGTTGGAACTGCCAAATTAACACTATCATACTATTTTGTTTTTTAATTTTAATTTTTTAAAAAA
TGTAAAATGTGCAATTTCAAGAGGAGAAACTTGAACACAAGGAGCAAAATCTATTTTTATAAC
ATCCTATTAAAAGCTTGCTTTACATAAAGATTTTGAAAGAATAGCATAAATACAAGATTTCTAT
TTTAATTGGATTCTTAGGGCTAATAAAATAATCAGCCTTAGCACTTATTTATTTATTTTTTTTGA
GAGGGAGTCTCGCTCTGTTGTCCATGCTGGAGTGCAGTGGCGTGATCTCGGCTCACTGCAA
GCTCCACCTCATGAGTTCACACCATTCTCCTGCCTCAGTCTCCCGAGTAGCTGGGACTCCAG
GCGCCCTCTACAAAGCCCGTCTAATTTTTTTTGTATTTTTAGTAGAGACAGGGTTTCACTGTG
TTAGCCAGGATGGTCTTGATCTCCTGACCTTGTGATCTGCCCGCCTCGGCCTCCCAAAGTG
CTGGGATTATAGGCTTGAGCCACTGCTCCCGGCCAGCACTTATTTTTATAATTCTTCATGATT
ACTGTGTTACTGTCCCATGGGCCGCCAGGGCCAGCTAGGTTGGCCACTCCCTCTCTGCGCG
CTCGCTCGCTCACTGAGGCCGGGCGACCAAAGGTCGCCCGACGCCCGGGCTTTGCCCGG
GCGGCCTCAGTGAGCGAGCGAGCGCGCAGAGAGGGAGTGGCCAACTCCATCACTAGGGGT
TCCTCCTAGCACGCGTAGAATCAGAAAATCTGAGAAGAAAACTTAAAAACCTTTGAATCTAAC
CTACTCAAATAAACTTTGAATATATTTATTGAAATATTTAATTTGTTTATTTTTTATTTTTTTATTT
TAGAGACAAGGTCTCACTAAAGTGCAGGCTAGAGTGCAGTGACACAATCATGGCTATGGCT
CACTGCAACCTCAAACTCCTGGGCTCAAGCGATTCTGTTGCCTTAGGTTCGCCAGGAGCTG
GCACTACAGGTGCCACCACACCTGGCTTTTTTGTTTTGTTTTTTTTGGGTAGAGAAGGGGATT
TGGTATGTTGCTTAGGCTGATCTTGAACTAGCCTCACGCAATCCTGCTTCGGCTTTCCAAAAT
GTTGGGATTATAGGCATGAGCCATGCGCCTGACCTTGATTACCTCGATGATGTGTATGTCAT
ACATTGGAGGGCAAAGACATCTCTGAATTCCTCACAACAGCCATGGCAGGGGGCAGGTACC
CATGTTACAGATGACAGACTGATGCATAGAGAAGTTAAGTCGTGGGGAGTTTACTTTCTCCT
AAATTGTCCTGTTACTAGATGAATTTGTTTTTGTTTCATTTTGTTTTGTTTTTGAGACAGAGTCT
CATGTTGTCACCCAGACTGGAGTGCAGTGGCTCCATCTCGGCTCACTATAGCCTCCGCCTC
CTGAGTTCAAGTGATTCTCCTGCCTCAGCCTCCCAAGTAGCTGGGTGCATGCCACCACGCC
TGGCTAATTTTTGTATTTTTAGTATAGATGGGGTTTCACCATGTTGGCCAGACTGGTCTCGAA
CTCCTGACCTCAAGTGATCCACCCCGCGCTTAGCCTCCCAAAGTGCTGGGATTATAGGCGT
GAGCCACCACACCAGGCCATGAATTTGTTTTCAATATTTATTTATTTTGTATTTTCTATTTTTGA
GATGGAGTCTCGCTCTGCTGTCCAGGCTAGAGTGCAGTGGTGTGATCTTGGCTCACTGTAG
CCTCCACCTCCTGAGTTCAAGTGATTCTCCTGCCTTAGCCTCCCGAGTAGCTGGGATTACAG
GCGCCCACCATCACACCCGGCTAATTTTTGTATTTTTAGTAGAGATGGGGTTTCACCATGTT
GGCCACGCTGGTCTCAAACTGACCTCAATTGATCCACCCACCTTGACCTCCCAAAGTGCTG
GGATTACAGACCTGAGCCACAGCGCCCAGCCCTTCAATATTTATTTAAATTTGCCTGCTGGC
TAACTTCTCATTGCACCTGGGCTCTAGTGTAATTAAATTACTTCATTCTCTTTTTAAAACTTTTT
ACTTTTTTCTTTTTTGTGTTTTTCATTCTCTTATCTACGAGAGCCACAATACTTGAAGACACCA
ATTGATACCCCTTAGTCACATCTGAGCTAAACACTTTCAGTTCCTACAGCTGTTTCTTAATCTT
AGGTCACATGGTTTCTTCCCATGCTGTTCTTCCCAGACAGCATTTTTTTTTTTTTTGAGAGTCT
CACTCTGTTGCCCAGGCTGGAGTACAGTAGCACAATCTCAGTTTACTGCAACCTCTGCCTTC
CAGGTTCAGGTGATTCTCCTGCTTCAGCCTCCTGAGTAGCTGGGACTACAGGAGCGTGCCA
CCACGCCCGGCTAATTTTTGTATTTTTAGTAGAGACAGGGTTTCACCATGTTGGCCAGGCTG
GTCTCGAACTCCTTACCTTGTGATCCGCCTGTCTCGGCCTCCCAAAGTGGTGGGATTACAG
GTGTGAGCCACCACGCCTGGTTCTTACATTTATTTTGGAATAAATTTAGATACACAGAAAAGT
TGCAAAGATAAGAGTTTCCATATAACCCTCACCCAGTTTGCCTTCCCTAATGTTCGCGTGGG
GTTGGGGTTGCGCCTTTTCCAAGGCAGCCCTGGGTTTGCGCAGGGACGCGGCTGCTCTGG
GCGTGGTTCCGGGAAACGCAGCGGCGCCGACCCTGGGTCTCGCACATTCTTCACGTCCGT
TCGCAGCGTCACCCGGATOTTCGCCGCTACCCTTGTGGGCCCCCCGGCGACGCTTCCTGC
TCCGCCCCTAAGTCGGGAAGGTTCCTTGCGGTTCGCGGCGTGCCGGACGTGACAAACGGA
AGCCGCACGTCTCACTAGTACCCTCGCAGACGGACAGCGCCAGGGAGCAATGGCAGCGCG
CCGACCGCGATGGGCTGTGGCCAATAGCGGCTGCTCAGCAGGGCGCGCCGAGAGCAGCG
GCCGGGAAGGGGCGGTGCGGGAGGCGGGGTGTGGGGCGGTAGTGTGGGCCCTGTTCCTG
CCCGCGCGGTGTTCCGCATTCTGCAAGCCTCCGGAGCGCACGTCGGCAGTCGGCTCCCTC
GTTGACCGAATCACCGACCTCTCTCCCCAGCTCTAAGGTAAATATAAAATTTTTAAGTGTATA
ATGTGTTAAACTACTGATTCTAATTGTTTCTCTCTTTTAGATTCCAACCTTTAGAACTGACCAC
CATGTGGACCCTGGGCAGGAGGGCTGTGGCTGGCCTGCTGGCCAGCCCCAGCCCTGCCCA
GGCCCAGACCCTGACCAGGGTGCCCAGGCCTGCTGAGCTGGCCCCCCTGTGTGGCAGGA
GGGGCCTGAGGACTGACATTGATGCCACCTGCACCCCCAGGAGGGCCAGCAGCAACCAGA
GGGGCCTGAACCAGATCTGGAATGTGAAGAAGCAGTCTGTGTACCTGATGAACCTGAGGAA
GTCTGGCACCCTGGGCCACCCTGGCAGCCTGGATGAAACCACCTATGAGAGGCTGGCTGA
GGAAACCCTGGACAGCCTGGCTGAGTTCTTTGAGGACCTGGCTGACAAGCCCTACACCTTT
GAGGACTATGATGTGAGCTTTGGCTCTGGGGTGCTGACTGTGAAGCTGGGGGGGGACCTG
GGCACCTATGTGATCAACAAGCAGACCCCCAACAAGCAGATCTGGCTGAGCAGCCCCAGCT
CTGGCCCCAAGAGATATGACTGGACTGGCAAGAACTGGGTGTACAGCCATGATGGGGTGAG
CCTGCATGAGCTGCTGGCTGCTGAGCTGACCAAGGCCCTGAAGACCAAGCTGGACCTGAG
CAGCCTGGCCTACTCTGGCAAGGATGCCTGAGATATCGCTTTATTTGTGAAATTTGTGATGC
TATTGCTTTATTTGTAACCATTATAAGCTGCAATAAACAAGTTAACAACAACAATTGCATTCAT
TTTATGTTTCAGGTTCAGGGGGAGGTGTGGGAGGTTTTTTAAAGCGGCCAACATCTTACATT
ATCATGGTACATTTGTCAAAACTAAGACACTTTTTTTTCTAATAAAAATAATAGAGATGAGGTC
TCACTATATTGTCCAGGCTGGTCTCAAACTTTGAGCTCAAGCAGTCCTCCCACCTCCACCTC
CCAAAGTGCTGGGATTACAGGCATGAACCACCACACCCAGCCTACATTGTTATGTTACTACT
CTCCAGACTATTCAGATTTCACCAATTTTTCTATCCATAGCCTTTTTTTCTGTTTCAGGATCCA
ATTCAGGACACCATACCACTGTATGGTTAATTTTAAAAACTCAAATGTTGGGAGTTTGCTTTAT
CACCGCTGTATCTTATCATCTCGTTAGATAATTTTGTATCTGGAGCCTGCTCTCTAGCATGAA
TAAGTAAAAATGTGGACTTTGTGATTTACAGATCTGATATTTATGTTTTTGTGTTTTTACTTTAA
GTACTAGACAAAGTAAATCTAAGAGGTCTGTGGTACTGCACTAGAGAATTGTTACTTGATTAT
GGTGGGCCAAGTGGTTGAAAATTTACCTAACAATACTATAACATAGGCCATATTTCATAATTT
TAATCACAAGGCAATTGTGAAAATTTTATCAAATGTTTAATAAAAGCAAGGTGAAGAAGGTGA
TAGCTTTAAATTTACTTGCCATTTTTGGGCACACAAAAGTAATTTGCTCTGCCACTTAGAGTTA
TAAGGTCAAAGTGGGAGTAAATAATCTTTGTATTAGGAATGCGGCCGCTCTAGGAGGAACCC
CTAGTGATGGAGTTGGCCACTCCCTCTCTGCGCGCTCGCTCGCTCACTGAGGCCGCCCGG
GCAAAGCCCGGGCGTCGGGCGACCTTTGGTCGCCCGGCCTCAGTGAGCGAGCGAGCGCG
CAGAGAGGGAGTGGCCAACCTAGAGGCCGCCAGGGCCATATTTCTCAATTTTTAAATTTTTC
AAAAAAATTAATCCTTAATGTGCATATTTTTGAATTGTTAATATAACTTTTTGAGGTGATGTCTT
CATGTGTTTCAACTACTTAAAAACTTTTAAACAGTATATAATAAAAAATCTTCCAGGCCACTCA
CACCTGTAATCCCAGCACTTTGGGAGGCTGAGGTGGGCAGATCACCTGAGGGCAGGAGTTC
GAGACCAGCCTGGCCAATATATATATATTCATATATTCATATATATATATATATTCATATATTCA
TATATATATATTCATATATTCATATATATATATATATATATATATAGCAAAACCTCATCTCTAATA
AAATACAAAAATTAGCTGAGCGTGGTGATGGATGCCTGTAGTCCCAGCTACTCGGGAGGCT
GAGGCAGGAGAATCTCTTGAACCTGGGAGGTGGAGGTTGCAGTGAGCTGAGATGGTGCCA
CTGCCCTCCAGCCTGAGTGACAGAGCGAGACTCGGTCTCCAAAAAAAAACAACAAAAAAATC
TTCCATCCTTGTCTCCCATCCACCCCTTCCCCCCAGCATGTACTTGCAGACTTTATGCATATA
CAGTGAGTACTGTATATACACAAATAATAAAAAAATCATATATATAATATATGTAATTCCCCTTT
ACATGAAAGGTAGCACACTGGTCTGTACAGTCTGTCTGCACTGTGCTATTTCACTTTATATTT
TTATAGTTTGACAGAGTTCTAACATTTCTTTTTTTTTTTTTTTAACAGAGTCTTGTTCCTGATTG
TTAAATTTTAAAGCATCCTAAAGTTTGGTTTCACACTTGAATGAATACCATGTAAGGATTCACT
TACATAGATGTGGTTGCCTGAATCTTAAGAATAAAATAACATTGTTTGTATTTATTTAAATTAG
TGTTCCTTTTATGGTTTGCCTGAAAGCACAACAAAATCCTCACCAAGATATTACAATTATGACT
CCCATACAGGTAAACTGTTTAGAGATTGGCAAGCACCTTTTAATGAAAGGAGTCAGCCAGCT
TAGTGTGCAGTATTTATTTCTGCCGGAAGAGGGAGCTTCAGGGACAGACTTTGGTTTAGTCA
TGAAGCCTCCAGCACTCCCAAGCGGTTGTGGTTGACCAAGCAATTTATGCTTTTACCTTTCTA
CTTCCAGAGGCTTGTTTACTTATCAGTAAGCATTAATTTAGTGTCCCCTCAGATGCCTTTTAC
TTTCTTCTTTTCTGCCTAGAATAAGCTGCTCTTCCAATTTTGCAGCTACATGTTTCCACCCCA
GTTGGAATTTCTCCATAACATCCATTGTAGCTATCCTTCAATCTACAGCCTCTATTTCCTGTTA
TAGCTGGTCAGGTCTAATCCCTCAAAATACTCTGTCCCCTGCTTCCCTTATCTGCTGGCCAC
CTTTTTCCCCCACATACACACTGCCATGTCCCACCCTTCACTCAAGTTGTTCCCTGCCACCTC
AACAAATTTAAGTCCATAAAACCATCCAATGGGCTCGAGCCCTGCAGGATCATTGTCACATG
TGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAAGCCCAATCTGAATAATGTTA
CAACCAATTAACCAATTCTGATTAGAAAAACTCATCGAGCATCAAATGAAACTGCAATTTATTC
ATATCAGGATTATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGACTCGTCCAA
CATCAATACAACCTATTAATTTCCCCTCGTCAAAAATAAGGTTATCAAGTGAGAAATCACCAT
GAGTGACGACTGAATCCGGTGAGAATGGCAAAAGTTTATGCATTTCTTTCCAGACTTGTTCA
ACAGGCCAGCCATTACGCTCGTCATCAAAATCACTCGCATCAACCAAACCGTTATTCATTCG
TGATTGCGCCTGAGCGAGACGAAATACGCGATCGCTGTTAAAAGGACAATTACAAACAGGAA
TCGAATGCAACCGGCGCAGGAACACTGCCAGCGCATCAACAATATTTTCACCTGAATCAGGA
TATTCTTCTAATACCTGGAATGCTGTTTTTCCGGGGATCGCAGTGGTGAGTAACCATGCATC
ATCAGGAGTACGGATAAAATGCTTGATGGTCGGAAGAGGCATAAATTCCGTCAGCCAGTTTA
GTCTGACCATCTCATCTGTAACATCATTGGCAACGCTACCTTTGCCATGTTTCAGAAACAACT
CTGGCGCATCGGGCTTCCCATACAAGCGATAGATTGTCGCACCTGATTGCCCGACATTATC
GCGAGCCCATTTATACCCATATAAATCAGCATCCATGTTGGAATTTAATCGCGGCCTCGACG
TTTCCCGTTGAATATGGCTCATAACACCCCTTGTATTACTGTTTATGTAAGCAGACAGTTTTAT
TGTTCATGATGATATATTTTTATCTTGTGCAATGTAACATCAGAGATTTTGAGACACGGGCCA
GAGCTGCA

In some embodiments, the components are operably linked to each other in a 5′-to-3′ direction as ITR1-FXN-ITR2. In some embodiments, the components are operably linked to each other in a 5′-to-3′ direction as ITR1-P_{Euk}-FXN-ITR2. In some embodiments, the components are operably linked to each other in a 5′-to-3′ direction as ITR1-P_{Euk}-FXN-pA-ITR2. In some embodiments, the components are operably linked to each other in a 5′-to-3′ direction as ITR1-P_{Euk}-intron-FXN-pA-ITR2.

In some embodiments, the components are operably linked to each other in a 5′-to-3′ direction as ITR1-hFXNco-ITR2. In some embodiments, the components are operably linked to each other in a 5′-to-3′ direction as ITR1-promoter-hFXNco-ITR2. In some embodiments, the components are operably linked to each other in a 5′-to-3′ direction as ITR1-promoter-hFXNco-pA-ITR2. In some embodiments, the components are operably linked to each other in a 5′-to-3′ direction as ITR1-promoter-intron-hFXNco-pA-ITR2.

In some embodiments, the ITR1-FXN-ITR2 together are about 3.7 Kb to about 4.3 Kb (e.g., 3.8 Kb to about 4.2 Kb or about 3.9 Kb to about 4.1 Kb). For example, in some embodiments, the ITR1-FXN-ITR2 together are about 3.8 Kb to about 4.2 Kb in length. In some embodiments, the ITR1-FXN-ITR2 together are about 3.9 Kb to about 4.1 Kb in length. In some embodiments, the ITR1-FXN-ITR2 together are about 4.0 Kb in length.

In some embodiments, the ITR1-FXN-ITR2 together are about 3.7 Kb in length. In some embodiments, the ITR1-FXN-ITR2 together are about 3.8 Kb in length. In some embodiments, the ITR1-FXN-ITR2 together are about 3.9 Kb in length. In some embodiments, the ITR1-FXN-ITR2 together are about 4.0 Kb in length. In some embodiments, the ITR1-FXN-ITR2 together are about 4.1 Kb in length. In some embodiments, the ITR1-FXN-ITR2 together are about 4.2 Kb in length. In some embodiments, the ITR1-FXN-ITR2 together are about 4.3 Kb in length.

In some embodiments, the ITR1-hFXNco-ITR2 together are about 3.7 Kb to about 4.3 Kb (e.g., 3.8 Kb to about 4.2 Kb or about 3.9 Kb to about 4.1 Kb). For example, in some embodiments, the ITR1-hFXNco-ITR2 together are about 3.8 Kb to about 4.2 Kb in length. In some embodiments, the ITR1-hFXNco-ITR2 together are about 3.9 Kb to about 4.1 Kb in length. In some embodiments, the ITR1-hFXNco-ITR2 together are about 4.0 Kb in length

In some embodiments, the ITR1-hFXNco-ITR2 together are about 3.7 Kb in length. In some embodiments, the ITR1-hFXNco-ITR2 together are about 3.8 Kb in length. In some embodiments, the ITR1-hFXNco-ITR2 together are about 3.9 Kb in length. In some embodiments, the ITR1-hFXNco-ITR2 together are about 4.0 Kb in length. In some embodiments, the ITR1-hFXNco-ITR2 together are about 4.1 Kb in length. In some embodiments, the ITR1-hFXNco-ITR2 together are about 4.2 Kb in length. In some embodiments, the ITR1-hFXNco-ITR2 together are about 4.3 Kb in length.

In some embodiments, the length of the nucleic acids between and including ITR1 and ITR2 is about 3.9 Kb to about 4.7 Kb (e.g., about 4.0 Kb to about 4.6 Kb, about 4.1 Kb to about 4.5 Kb, about 4.2 Kb to about 4.4 Kb, or about 4.3 Kb). For example, in some embodiments, the length of the nucleic acids between and including ITR1 and ITR2 is about 4.0 Kb to about 4.7 Kb. In some embodiments, the length of the nucleic acids between and including ITR1 and ITR2 is about 4.1 Kb to about 4.7 Kb. In some embodiments, the length of the nucleic acids between and including ITR1 and ITR2 is about 4.2 Kb to about 4.7 Kb. In some embodiments, the length of the nucleic acids between and including ITR1 and ITR2 is about 4.3 Kb to about 4.7 Kb. In some embodiments, the length of the nucleic acids between and including ITR1 and ITR2 is about 4.4 Kb to about 4.7 Kb. In some embodiments, the length of the nucleic acids between and including ITR1 and ITR2 is about 4.5 Kb to about 4.7 Kb.

In some embodiments, the length of the nucleic acids between and including ITR1 and ITR2 is about 3.9 Kb. In some embodiments, the length of the nucleic acids between and including ITR1 and ITR2 is about 4.0 Kb. In some embodiments, the length of the nucleic acids between and including ITR1 and ITR2 is about 4.1 Kb. In some embodiments, the length of the nucleic acids between and including ITR1 and ITR2 is about 4.2 Kb. In some embodiments, the length of the nucleic acids between and including ITR1 and ITR2 is about 4.3 Kb. In some embodiments, the length of the nucleic acids between and including ITR1 and ITR2 is about 4.4 Kb. In some embodiments, the length of the nucleic acids between and including ITR1 and ITR2 is about 4.5 Kb. In some embodiments, the length of the nucleic acids between and including ITR1 and ITR2 is about 4.6 Kb. In some embodiments, the length of the nucleic acids between and including ITR1 and ITR2 is about 4.7 Kb.

In some embodiments, a plasmid (e.g., a transfer vector) comprises the AAV, as described herein. In some embodiments, the plasmid may contain one or more (e.g., two) spacer. Spacers can include naturally occurring nucleic acid molecules or synthetic nucleic acid molecules. Naturally occurring spacer molecules can be identified using on-line web tools, e.g., UCSC genome browser, and can be selected based on inherent features of the nucleic acid molecule, e.g., natural occurrence of a nucleic acid adjacent to a transcriptional start site. A spacer may be engineered to remove potentially negative features that may produce toxicity if introduced by a viral particle or suppress the functionality of the viral particle. Exemplary sources of toxicity and functionality suppressing features are found in contaminating nucleic acids frequently found adjacent to nucleic acids encoding for the viral genome to be packaged, These contaminating nucleic acids include, but are not limited to, prokaryotic (e.g., bacterial and baculoviral) nucleic acids (e.g., origin of replication, nucleic acids having greater than 2% CpG content, open reading frames, and transcription factor binding sites). In some embodiments, spacers are designed to minimize the inclusion of contaminating nucleic acids. In some embodiments, the one or more SS does not comprise an open reading frame that is greater than 100 amino acids in length. In some embodiments, the one or more spacers does not comprise prokaryotic transcription factor binding sites.

For example, in some embodiments, the plasmid contains two spacers, wherein the two spacers include a first spacer (SS1) and a second spacer (SS2). In some embodiments, SS1 is positioned 5′ to ITR1 and the SS2 is positioned 3′ to ITR2. In some embodiments, SS1 is about 1.0 Kb to about 5.0 Kb (e.g., about 1.5 to about 4.5 Kb, about 2.0 to about 4.0 Kb, or about 3.0 Kb) in length. In some embodiments, SS2 is about 1.0 Kb to about 5.0 Kb (e.g., about 1.5 to about 4.5 Kb, about 2.0 to about 4.0 Kb, or about 3.0 Kb) in length.

### Methods of Treatment

### Friedreich Ataxia

Friedreich ataxia is a neuro- and cardio-degenerative disease, which results from inherited alterations in the frataxin gene decreasing frataxin polypeptide expression. Identification of agents efficacious for the therapy of Friedreich ataxia has previously been hampered by the difficulty associated with achieving expression of therapeutically effective amounts of frataxin in affected tissues.

The invention is based in part on identification of a new use for several existing agents, that is, for treating Friedreich ataxia or other neurodegenerative disease. These agents include nucleic acid molecules described herein. These agents increase levels of frataxin protein, the hallmark deficiency of Friedreich ataxia, in a transgenic mouse model of Friedreich ataxia.

Patients amenable to treatment include individuals at risk of disease but not showing symptoms, as well as patients presently showing symptoms. Generally, the subject is homozygous for a mutation (a GAA expansion or point mutation) that inhibits or reduces the expression levels of frataxin. For subjects homozygous for a mutation in the frataxin gene that results in insufficient expression levels of the frataxin polypeptide, the risk of developing symptoms of Friedreich ataxia generally increases with age. Accordingly, in asymptomatic subjects homozygous for a mutation in the frataxin gene that results in insufficient expression levels of the frataxin polypeptide, in certain embodiments, prophylactic application is contemplated for subjects over 3 years of age, for example, in subjects over about 4, 5, 6, 7, 8, 9, 10, 11, 12, 13, 14, 15, 16, or years of age. Subjects with late or very late onset of disease, as described above can also be treated.

In some embodiments, the subject is exhibiting symptoms of Friedreich ataxia, for example, muscle weakness in the arms and legs, loss of coordination, loss of deep tendon reflexes, loss of extensor plantar responses, loss of vibratory and proprioceptive sensation, vision impairment, involuntary and/or rapid eye movements, hearing impairment, slurred speech, curvature of the spine (scoliosis), high plantar arches (pes cavus deformity of the foot), carbohydrate intolerance, diabetes mellitus, and heart disorders (e.g. atrial fibrillation, tachycardia (fast heart rate), hypertrophic cardiomyopathy, cardiomegaly, symmetrical hypertrophy, heart murmurs, and heart conduction defects).

In some embodiments, the disclosure provides a method of treating Friedreich Ataxia in a human patient in need thereof, the method including administering to the patient a therapeutically effective amount of a polynucleotide, described herein.

In some embodiments, the disclosure provides a method of increasing frataxin expression in a human patient diagnosed as having Friedreich Ataxia, the method including administering to the patient a therapeutically effective amount of a polynucleotide, described herein.

### Monitoring Efficacy

Clinical efficacy can be monitored using biomarkers among other methods. Measurable biomarkers to monitor efficacy include, but are not limited to, monitoring one or more of the physical symptoms of Friedreich ataxia, including muscle weakness in the arms and legs, loss of coordination, loss of deep tendon reflexes, loss of extensor plantar responses, loss of vibratory and proprioceptive sensation, vision impairment, involuntary and/or rapid eye movements, hearing impairment, slurred speech, curvature of the spine (scoliosis), high plantar arches (pes cavus deformity of the foot), carbohydrate intolerance, diabetes mellitus, and heart disorders (e.g., atrial fibrillation, tachycardia (fast heart rate), hypertrophic cardiomyopathy, cardiomegaly, symmetrical hypertrophy, heart murmurs, and heart conduction defects). Observation of the stabilization, improvement and/or reversal of one or more symptoms indicates that the treatment or prevention regime is efficacious. Observation of the progression, increase or exacerbation of one or more symptoms indicates that the treatment or prevention regime is not efficacious. A preferred biomarker for assessing treatment in Friedreich ataxia is a level of frataxin. This marker is preferably assessed at the protein level, but measurement of mRNA encoding frataxin can also be used as a surrogate measure of frataxin expression. Such a level can be measured in a blood sample. Such a level is reduced in subjects with Friedreich ataxia relative to a control population of undiseased individuals. Therefore, an increase in level provides an indication of a favorable treatment response, whereas an unchanged or decreasing levels provides an indication of unfavorable or at least non-optimal treatment response.

Efficacy can also be determined by determining the level of sclerosis and/or degeneration of dorsal root ganglia, spinocerebellar tracts, lateral corticospinal tracts, and posterior columns. This may be accomplishing using medical imaging techniques, e.g., magnetic resonance imaging or tomography techniques, e.g., computed tomography (CT) scan or computerized axial tomography (CAT) scan. Subjects who maintain the same level or a reversal of sclerosis and/or degeneration indicate that the treatment or prevention regime is efficacious. Conversely, subjects who show a higher level or a progression of sclerosis and/or degeneration indicate that the treatment or prevention regime has not been efficacious.

In certain embodiments, the monitoring methods can entail determining a baseline value of a measurable biomarker or disease parameter in a subject before administering a dosage of the polynucleotides or plasmids (e.g., a viral vector) described herein, and comparing this with a value for the same measurable biomarker or parameter after a course of treatment.

In other methods, a control value (i.e., a mean and standard deviation) of the measurable biomarker or parameter is determined for a control population. In certain embodiments, the individuals in the control population have not received prior treatment and do not have Friedreich ataxia, nor are at risk of developing Friedreich ataxia. In such cases, if the value of the measurable biomarker or clinical parameter approaches the control value, then treatment is considered efficacious. In other embodiments, the individuals in the control population have not received prior treatment and have been diagnosed with Friedreich ataxia. In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

In some embodiments, upon administering a nucleic acid molecule described herein, the patient displays a change in whole blood frataxin levels. For example, in some embodiments, the patient displays the change in whole blood frataxin levels by about 12 weeks after administration.

In some embodiments, upon administering a nucleic acid molecule described herein, the patient displays a reduction in Total Friedreich Ataxia Rating Scale (FARS) Score. For example, in some embodiments, the patient displays the reduction in Total FARS Score by about 12 weeks after administration.

### Methods of Measuring Gene Expression

The expression level of a gene expressed by a single cell or type of cell (e.g., a cell belonging to a certain tissue) can be ascertained, for example, by evaluating the concentration or relative abundance of RNA transcripts (e.g., mRNA)) derived from transcription of a gene of interest. Additionally, or alternatively, gene expression can be determined by evaluating the concentration or relative abundance of protein produced by transcription and translation of a gene of interest. Protein concentrations can also be assessed using functional assays, such as enzymatic assays or gene transcription assays in the event the gene of interest encodes an enzyme or a modulator of transcription, respectively. The sections that follow describe exemplary techniques that can be used to measure and rank the expression levels of genes in a cell, cell type, or population of cells of interest, for instance, at the level of a single cell or a population of cells. Expression of genes in a sample can be analyzed by a number of methodologies, many of which are known in the art and understood by the skilled artisan, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### (i) Nucleic Acid Detection

Nucleic acid-based datasets suitable for analysis of target cell-specific gene expression can have the form of a gene expression profile, which represents the identity of genes expressed in a cell of interest and the extent to which the gene is expressed, which can be used to determine the ranked order of gene expression levels within a cell, cell type, or population of cells of interest. Such profiles may include whole transcriptome sequencing data (e.g., RNA-Seq data), panels of mRNAs, noncoding RNAs, or any other nucleic acid sequence that may be expressed from genomic DNA. Other nucleic acid datasets suitable for use with the methods described herein may include expression data collected by imaging-based techniques (e.g., Northern blotting or Southern blotting known in the art). Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500, the disclosure of which is incorporated herein by reference. Typical protocols for evaluating the status of genes and gene products are found, for example in Ausubel et al., eds., 1995, Curr Protoc Mol Biol, Units 2 (Northern Blotting), 4 (Southern Blotting), 15 (Immunoblotting) and 18 (PCR Analysis), the disclosure of which is incorporated herein by reference.

Gene expression profiles to be analyzed in conjunction with the methods described herein may include, for example, microarray data or nucleic acid sequencing data produced by a sequencing method known in the art (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, mRNA expression levels may be determined using RNA-Seq (e.g., as described in Mortazavi et al., Nat. Methods 5:621-628 (2008), the disclosure of which is incorporated herein by reference in their entirety). RNA-Seq is a robust technology known in the art for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Gene expression levels may be determined using microarray-based platforms (e.g., single-nucleotide polymorphism (SNP) arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of one or more markers (e.g., genes). In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996) and in Heid et al., Genome Res. 6:986-994 (1996), the disclosures of each of which are incorporated herein by reference. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### (ii) Protein Detection

Gene expression can additionally be determined by measuring the concentration or relative abundance of a corresponding protein product encoded by a gene of interest. Protein levels can be assessed using standard detection techniques known in the art. Examples of protein expression analysis that generate data suitable for use with the methods described herein include, without limitation, proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including but not limited to monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference.

Mass spectrometry (MS) may be used in conjunction with the methods described herein to identify and characterize the gene expression profile of a single cell or multi-cell population. Any method of MS known in the art may be used to determine, detect, and/or measure a peptide or peptides of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79 (2009), the disclosure of which is incorporated herein by reference.

Prior to MS analysis, proteins in a sample can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for methods described herein allowing for ionization followed by fragmentation of a complex peptide sample, such as a sample obtained from a multi-cell population described herein. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place overtime. Signature MS/MS spectra may then be compared against a peptide sequence database (e.g., SEQUEST). Post-translational modifications to peptides may also be determined, for example, by searching spectra against a database while allowing for specific peptide modifications.

### Pharmaceutical Compositions

The compositions, nucleic acid molecules, and plasmids described herein can be formulated into pharmaceutical compositions for administration to a patient, such as a human patient exhibiting or at risk of Friedreich Ataxia, in a biologically compatible form suitable for administration in vivo. A pharmaceutical composition containing, for example, a nucleic acid molecule including one or more transgenes described herein, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., phosphate-buffered saline (PBS) and a nucleic acid. The sterile PBS is typically a pharmaceutical grade PBS.

In certain embodiments, pharmaceutical compositions include one or more composition or nucleic acid molecule and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, nucleic acid molecules may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including a nucleic acid molecule encompass any pharmaceutically acceptable salts of the inhibitor, esters of the inhibitor, or salts of such esters. In certain embodiments, pharmaceutical compositions including a nucleic acid molecule, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to a nucleic acid molecule, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intraocular (e.g., intravitreal), intravenous, subcutaneous, intramuscular, intrathecal, intracerebroventricular, intraparenchymal etc.). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Kits

The compositions described herein can be provided in a kit for use in treating Friedreich ataxia. The kit may include one or more compositions, nucleic acid molecules, plasmids, or pharmaceutical compositions as described herein. The kit can include a package insert that instructs a user of the kit, such as a physician, to perform any one of the methods described herein. The kit may optionally include a syringe or other device for administering the composition. In some embodiments, the kit may include one or more additional therapeutic agents.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Materials and Methods

Codon Optimization of the Frataxin Gene for Efficacious Protein Expression and Restoration of the Expansion of Intronic Trinucleotide Repeat GAA in Friedreich ataxia

The FXN isoform 1 gene sequence, excluding intronic DNA, is as follows:

(SEQ ID NO: 5)
AGTCTCCCTTGGGTCAGGGGTCCTGGTTGCACTCCGTGCTTTGCACAAAGCAGGCT
CTCCATTTTTGTTAAATGCACGAATAGTGCTAAGCTGGGAAGTTCTTCCTGAGGTCT
AACCTCTAGCTGCTCCCCCACAGAAGAGTGCCTGCGGCCAGTGGCCACCAGGGGT
CGCCGCAGCACCCAGCGCTGGAGGGGGGAGCGGGCGGCAGACCCGGAGCAGCAT
GTGGACTCTCGGGCGCCGCGCAGTAGCCGGCCTCCTGGCGTCACCCAGCCCAGC
CCAGGCCCAGACCCTCACCCGGGTCCCGCGGCCGGCAGAGTTGGCCCCACTCTGC
GGCCGCCGTGGCCTGCGCACCGACATCGATGCGACCTGCACGCCCCGCCGCGCA
AGTTCGAACCAACGTGGCCTCAACCAGATTTGGAATGTCAAAAAGCAGAGTGTCTAT
TTGATGAATTTGAGGAAATCTGGAACTTTGGGCCACCCAGGCTCTCTAGATGAGACC
ACCTATGAAAGACTAGCAGAGGAAACGCTGGACTCTTTAGCAGAGTTTTTTGAAGAC
CTTGCAGACAAGCCATACACGTTTGAGGACTATGATGTCTCCTTTGGGAGTGGTGTC
TTAACTGTCAAACTGGGTGGAGATCTAGGAACCTATGTGATCAACAAGCAGACGCCA
AACAAGCAAATCTGGCTATCTTCTCCATCCAGTGGACCTAAGCGTTATGACTGGACT
GGGAAAAACTGGGTGTACTCCCACGACGGCGTGTCCCTCCATGAGCTGCTGGCCG
CAGAGCTCACTAAAGCCTTAAAAACCAAACTGGACTTGTCTTCCTTGGCCTATTCCG
GAAAAGATGCTTGATGCCCAGCCCCGTTTTAAGGACATTAAAAGCTATCAGGCCAAG
ACCCCAGCTTCATTATGCAGCTGAGGTCTGTTTTTTGTTGTTGTTGTTGTTTATTTTTT
TTATTCCTGCTTTTGAGGACAGTTGGGCTATGTGTCACAGCTCTGTAGAAAGAATGT
GTTGCCTCCTACCTTGCCCCCAAGTTCTGATTTTTAATTTCTATGGAAGATTTTTTGG
ATTGTCGGATTTCCTCCCTCACATGATACCCCTTATCTTTTATAATGTCTTATGCCTAT
ACCTGAATATAACAACCTTTAAAAAAGCAAAATAATAAGAAGGAAAAATTCCAGGAGG
GAAAATGAATTGTCTTCACTCTTCATTCTTTGAAGGATTTACTGCAAGAAGTACATGA
AGAGCAGCTGGTCAACCTGCTCACTGTTCTATCTCCAAATGAGACACATTAAAGGGT
AGCCTACAAATGTTTTCAGGCTTCTTTCAAAGTGTAAGCACTTCTGAGCTCTTTAGCA
TTGAAGTGTCGAAAGCAACTCACACGGGAAGATCATTTCTTATTTGTGCTCTGTGAC
TGCCAAGGTGTGGCCTGCACTGGGTTGTCCAGGGAGACCTAGTGCTGTTTCTCCCA
CATATTCACATACGTGTCTGTGTGTATATATATTTTTTCAATTTAAAGGTTAGTATGGA
ATCAGCTGCTACAAGAATGCAAAAAATCTTCCAAAGACAAGAAAAGAGGAAAAAAAG
CCGTTTTCATGAGCTGAGTGATGTAGCGTAACAAACAAAATCATGGAGCTGAGGAG
GTGCCTTGTAAACATGAAGGGGCAGATAAAGGAAGGAGATACTCATGTTGATAAAGA
GAGCCCTGGTCCTAGACATAGTTCAGCCACAAAGTAGTTGTCCCTTTGTGGACAAGT
TTCCCAAATTCCCTGGACCTCTGCTTCCCCATCTGTTAAATGAGAGAATAGAGTATG
GTTGATTCCCAGCATTCAGTGGTCCTGTCAAGCAACCTAACAGGCTAGTTCTAATTC
CCTATTGGGTAGATGAGGGGATGACAAAGAACAGTTTTTAAGCTATATAGGAAACAT
TGTTATTGGTGTTGCCCTATCGTGATTTCAGTTGAATTCATGTGAAAATAATAGCCAT
CCTTGGCCTGGCGCGGTGGCTCACACCTGTAATCCCAGCACTTTTGGAGGCCAAGG
TGGGTGGATCACCTGAGGTCAGGAGTTCAAGACCAGCCTGGCCAACATGATGAAAC
CCCGTCTCTACTAAAAATACAAAAAATTAGCCGGGCATGATGGCAGGTGCCTGTAAT
CCCAGCTACTTGGGAGGCTGAAGCGGAAGAATCGCTTGAACCCAGAGGTGGAGGT
TGCAGTGAGCCGAGATCGTGCCATTGCACTGTAACCTGGGTGACTGAGCAAAACTC
TGTCTCAAAATAATAATAACAATATAATAATAATAATAGCCATCCTTTATTGTACCCTT
ACTGGGTTAATCGTATTATACCACATTACCTCATTTTAATTTTTACTGACCTGCACTTT
ATACAAAGCAACAAGCCTCCAGGACATTAAAATTCATGCAAAGTTATGCTCATGTTAT
ATTATTTTCTTACTTAAAGAAGGATTTATTAGTGGCTGGGCATGGTGGCGTGCACCT
GTAATCCCAGGTACTCAGGAGGCTGAGACGGGAGAATTGCTTGACCCCAGGCGGA
GGAGGTTACAGTGAGTCGAGATCGTACCTGAGCGACAGAGCGAGACTCCGTCTCAA
AAAAAAAAAAAAGGAGGGTTTATTAATGAGAAGTTTGTATTAATATGTAGCAAAGGCT
TTTCCAATGGGTGAATAAAAACACATTCCATTAAGTCAAGCTGGGAGCAGTGGCATA
TACCTATAGTCCCAGCTGCACAGGAGGCTGAGACAGGAGGATTGCTTGAAGCCAGG
AATTGGAGATCAGCCTGGGCAACACAGCAAGATCCTATCTCTTAAAAAAAGAAAAAA
AAACCTATTAATAATAAAACAGTATAAACAAAAGCTAAATAGGTAAAATATTTTTTCTG
AAATAAAATTATTTTTTGAGTCTGATGGAAATGTTTAAGTGCAGTAGGCCAGTGCCAG
TGAGAAAATAAATAACATCATACATGTTTGTATGTGTTTGCATCTTGCTTCTACTGAAA
GTTTCAGTGCACCCCACTTACTTAGAACTCGGTGACATGATGTACTCCTTTATCTGG
GACACAGCACAAAAGAGGTATGCAGTGGGGCTGCTCTGACATGAAAGTGGAAGTTA
AGGAATCTGGGCTCTTATGGGGTCCTTGTGGGCCAGCCCTTCAGGCCTATTTTACTT
TCATTTTACATATAGCTCTAATTGGTTTGATTATCTCGTTCCCAAGGCAGTGGGAGAT
CCCCATTTAAGGAAAGAAAAGGGGCCTGGCACAGTGGCTCATGCCTGTAATCCCAG
CACTTTGGGAGGCTGAGGCAAGTGTATCACCTGAGGTCAGGAGTTCAAGACCAGCC
TGGCCAACATGGCAAAATCCCGTCTCTACTAAAAATATTAAAAAATTGGCTGGGCGT
GGTGGTTCGTGCCTATAATTTCAGCTACTCAGGAGGCTGAGGCAGGAGAATCGCTG
TAACCTGGGGGGGGAGGTTGCAGTGAGACGAGATCATGCCACTTCACTCCAGCCT
GGCCAACAGAGCCATACTCCGTCTCAAATAAATAAATAAATAAATAAAGGGACTTCA
AACACATGAACAGCAGCCAGGGGAAGAATCAAAATCATATTCTGTCAAGCAAACTGG
AAAAGTACCACTGTGTGTACCAATAGCCTCCCCACCACAGACCCTGGGAGCATCGC
CTCATTTATGGTGTGGTCCAGTCATCCATGTGAAGGATGAGTTTCCAGGAAAAGGTT
ATTAAATATTCACTGTAACATACTGGAGGAGGTGAGGAATTGCATAATACAATCTTAG
AAAACTTTTTTTTCCCCTTTCTATTTTTTGAGACAGGATCTCACTTTGGCACTCAGGC
TGGAGGACAGTGGTACAATCAAAGCTCATGGCAGCCTCGACCTCCCTGGGCTTGGG
CAATCCTCCCACAGGTGTGCACCTCCATAGCTGGCTAATTTGTGTATTTTTTGTAGA
GATGGGGTTTCACCATGTTGCCCAGGCTGGTCTCTAACACTTAGGCTCAAGTGATC
CACCTGCCTCGTCCTCCCAAGATGCTGGGATTACAGGTGTGTGCCACAGGTGTTCA
TCAGAAAGCTTTTTCTATTATTTTTACCTTCTTGAGTGGGTAGAACCTCAGCCACATA
GAAAATAAAATGTTCTGGCATGACTTATTTAGCTCTCTGGAATTACAAAGAAGGAATG
AGGTGTGTAAAAGAGAACCTGGGTTTTTGAATCACAAATTTAGAATTTAATCGAAACT
CTGCCTCTTACTTGTTTGTAGACACTGACAGTGGCCTCATGTTTTTTTTTTTTTTAATC
TATAAAATGGAGATATCTAACATGTTGAGCCTGGGCCCACAGGCAAAGCACAATCCT
GATGTGAGAAGTACTCAGTTCATGACAACTGTTGTTCTCACATGCATAGCATAATTTC
ATATTCACATTGGAGGACTTCTCCCAAAATATGGATGACGTTCCCTACTCAACCTTGA
ACTTAATCAAAATACTCAGTTTACTTAACTTCGTATTAGATTCTGATTCCCTGGAACCA
TTTATCGTGTGCCTTACCATGCTTATATTTTACTTGATCTTTTGCATACCTTCTAAAAC
TATTTTAGCCAATTTAAAATTTGACAGTTTGCATTAAATTATAGGTTTACAATATGCTT
TATCCAGCTATACCTGCCCCAAATTCTGACAGATGCTTTTGCCACCTCTAAAGGAAG
ACCCATGTTCATAGTGATGGAGTTTGTGTGGACTAACCATGCAAGGTTGCCAAGGAA
AAATCGCTTTACGCTTCCAAGGTACACACTAAGATGAAAGTAATTTTAGTCCGTGTCC
AGTTGGATTCTTGGCACATAGTTATCTTCTGCTAGAACAAACTAAAACAGCTACATGC
CAGCAAGGGAGAAAGGGGAAGGAGGGGCAAAGTTTTGAAATTTCATGTAAATTTAT
GCTGTTCAAAACGACGAGTTCATGACTTTGTGTATAGAGTAAGAAATGCCTTTTCTTT
TTTGAGACAGAGTCTTGCTCTGTCACCCAGGCTGGAGTGCAGTGGCACGATCTGGG
CTCACTACAACCTCCGCCTCCTGGGTTCAAGCAATTCTCTGCCTCAGCCTCCCGAGT
AGCTGGGATTACAGGTGCCTGCCACCACACCCGGCTAATTTTTGTATTTTTAGTAGA
GACGGGGTTTCACCATCATGGCCAGGCTGGTCTTGAACTCCTGACCTAGTAATCCA
CCTGCCTCCGCCTCCCAAAGTGCTGGGATTACAGGCGTGAGCCACTGCACCCAGC
CAGAAATGCCTTCTAATCTTTGGTTTATCTTAATTAGCCAGGACACTTGGAGTGCATC
CCGAAGTACCTGATCAGTGGCCCCTTTGGAATGTGTAAAACTCAGCTCACTTATATC
CCTGCATCCGCTACAGAGACAGAATCCAAGCTCATATGTTCCATCTTCTCTGGCTGT
ATAGTTTAAGGAATGGAAGGCACCAGAACAGATTTATTGAAATGTTTATTAGCTGAAG
ATTTATTTAGACAGTTGAGGAAAACATCAGCACCCAGCAGTAAAATTGGCTCTCAAA
GATTTTCTTCTCCTGTGGAAAGTCAGACCTCTGAGGCCCCATCCAGGTAGAAGTACT
AGTGCAAGAAGGGCCTCTGCTGTCCACTTGTGTTTCTGTGATCTGTGGGAACATTGT
TAACGCCACATCTTGACCTCAAATTGTTTAGCTCCTGGCCAGACACGGTGGCTCACA
CCTGTAATCCCAGCACTTTGAGAGGCTGAGGCAGGTGGATCACCTGAGGTTAGGAG
TTCGAGGCCAGCCTGGTCAACATGGTAAAACCCCGCCTCTACTAAAAATACAAAAAT
TAGCTGGCCGTAGTGGCGCACGCCTGTTATCCCAGCTACTCGGGAGGCTGAGGCA
GGAGAATTGCTTGAACCTGGGTGGTGGAGGTTGCAGTGAGCCGAGATTACACCACT
GCACTCCAGCCTGGGTGACAAGAGGGAAACTCCATTAAAAAAATGTAATTCCCGTGT
CTGCCATCTTAAGTGTAAAGGTGGCTAAATTATATAGAAAAATAAGACAATATCATTT
CCCAATTACATTCCTTTCCTACCGCACTCTATGATGCTAGCTGAGATTTTTCCAAAAG
AAAATGGCTTAAATAAAACCCTAAGAGAAAGAAAAACTTTAAATCCCTCCAAAGCTCA
AAAGTAATAGAAACAGATGAGTTTGGAGTCAGGATTTCTCTGTAAGATTGCCTAGGC
TGTGTACTGCACATCTCCAGGTGCCACTGTTGACAGAGATTATAACTACAATGTGAA
GTGAATGGTGCCACTGACAGTTATGCAAACCGTCCAGAGCATAGCCACCTGATCCT
GCTGGGATTCCTCTTGCCAGTCCATCAGCAGTTCCCCTTGAAAGTTTCACCAAACAT
CCCTTAAATCTGCCCTCTCCTGCCCGTCCCCAGTGGAGGTCCTCATCATTTTTCACC
TGCATTTTTGCAGGAGCTTTCTTATATCCACCTTCCTCCTTTTCTCTCAGCCCATCAT
CTAGCTACACAGTCTCCAGGGTAAGCTTTCAGAAAGGCAATCTCTTGTCTGTAAAAC
CTAAGCAGGACCAAGGCCAAGTTTCTTAGCCTGAAAAATGTGCTTTTCTGACTGAAC
TGTTCAGGCACTGACTCTACATATAATTATGCTTTTCTACCCCCTCACACTCAACACT
TTGACTCCAGCAATCCCAAATCCCCAGATCCCTAAGTGTGCTGTGCTATTTTCACGT
GGCTCTCAGACTTGGCCAGTGCTGTTTCCATTTTGGTCTTTATTCCCCACATCTCTG
CCTGGGGGGTAGATTCTACCCTGAAAAATGTTCTTGGCACAGCCTTGCAAACTCCTC
CTCCACTCAGCCTCTGCCTGGATGCCCTTGATTGTTCCATGTCCTCAGCATACCATG
TTTGTCTTTCCCAGCACTGACCTACCATGTGTCACCCCTGCTTGGCTGTACCTTCCA
TGAGGCTAGGACTATGTGTCTCCTTTGTTGACTGCTGTTGCCCTAGCATCTTGCACA
GTTCCTTGCACACAATTAGAGCTCTATAAATGTCAAATAAATGTGTTATAATTATATGT
TTAAGATAGTTGTTCAAATAAACTCTAAATAACCCCAAC.

The frataxin isoform 1 amino acid sequence is as follows:

(SEQ ID NO: 3)
MWTLGRRAVAGLLASPSPAQAQTLTRVPRPAELAPLCGRRGLRTDIDAT
CTPRRASSNQRGLNQIWNVKKQSVYLMNLRKSGTLGHPGSLDETTYERL
AEETLDSLAEFFEDLADKPYTFEDYDVSFGSGVLTVKLGGDLGTYVINK
QTPNKQIWLSSPSSGPKRYDWTGKNWVYSHDGVSLHELLAAELTKALKT
KLDLSSLAYSGKDA

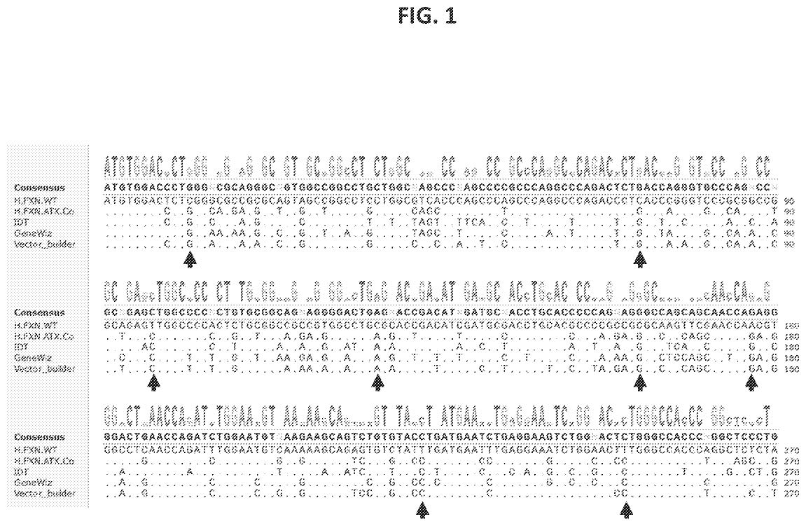

Analysis of SEQ ID NO: 5 reveals specific codon preferences for various amino acids throughout the gene. Inspection of the codon frequencies reveals that for certain amino acids, a particular codon is predominantly used while other codons are used less frequently or not at all. One of skill in the art can rationally design variants of the FXN gene, for example to increase protein stability, reduce local GC content, and/or reduce mRNA secondary structure and unstable motifs. Codon-optimized design variants of the FXN gene that contain reduced CpG content and/or reduced homopolymer content so as to enhance the translation of frataxin. For instance, one of skill in the art can manipulate the frataxin-encoding gene sequence by incorporating codon substitutions that diminish the CpG content and/or homopolymer content of the designed FXN gene (FIG. 1). For instance, in the FXN isoform 1 gene sequence above, there are instances of the homopolymer GGGGGG. Homopolymers can be a site of frameshift mutations in the formation of an mRNA transcript and/or during the translation process. If this homopolymer sequence remains in the codon-optimized FXN gene even after minimizing the sequence identity of the gene relative to endogenously expressed genes in the target cell, one of skill in the art can incorporate further mutations that interrupt this homopolymer while preserving the identity of the encoded protein. Alternatively, if the homopolymer encodes amino acid residues that are not essential for protein function (for instance, if the encoded amino acids are not present within the active site that mediates function of frataxin (e.g., the active site that mediates assembly of iron-sulfur clusters), one of skill in the art can incorporate codon substitutions that interrupt the homopolymer and that introduce a conservative substitution into the encoded protein at the site of the corresponding amino acid.

Furthermore, the final codon-optimized gene may exhibit at least 95% sequence identity to an endogenous RNA molecule encoding the frataxin variant 1 (SEQ ID NO: 5). For example, the final codon-optimized gene may exhibit at least 96%, 97%, 98%, or 99% sequence identity to an endogenous RNA molecule encoding the frataxin variant 1 (SEQ ID NO: 5). In another example, the final codon-optimized gene may have a nucleic acid sequence that is identical to the nucleic sequence of SEQ ID NO: 1. In some embodiments, the codon-optimized human FXN gene may include a modified Kozak sequence including the nucleic acid sequence: CCACCATG (SEQ ID NO: 6), relative to nucleic acids 8-15 of SEQ ID NO: 5.

Once designed, the final codon-optimized gene can be prepared, for instance, by solid phase nucleic acid procedures known in the art. Techniques for the solid phase synthesis of polynucleotides are known in the art and are described, for instance, in U.S. Pat. No. 5,541,307, the disclosure of which is incorporated herein by reference as it pertains to solid phase polynucleotide synthesis and purification. Additionally, the prepared gene can be amplified, for instance, using PCR-based techniques described herein or known in the art, and/or by transformation of DH5a E. coli with a plasmid containing the designed gene. The bacteria can subsequently be cultured so as to amplify the DNA therein, and the gene can be isolated plasmid purification techniques known in the art, followed optionally by a restriction digest and/or sequencing of the plasmid to verify the identity codon-optimized gene.

### Immunofluorescence

Biopsied heart was fixed in 10% formalin overnight, then preserved in 70% ethanol. The tissue was embedded in paraffin and cut in 5-μm sections.

For immunofluorescence analysis, tissues were permeabilized and slides were washed and blocked for 30 min with 5% goat serum. Sections were incubated with primary antibody anti-frataxin (Abcam 175402; 2 μg/mL; recognizes human, mouse, and rat) for 1 hour at room temperature. Slides were incubated with secondary goat anti-rabbit antibody (Thermo A11008; AlexaFluor 488 conjugate, 4 μg/mL) for 30 min. Assessments included detailed quantification of frataxin expression.

### Codon-Optimized Human Frataxin Constructs

PCR primers were designed to bind to and amplify the 633 basepair of the FXN nucleic acid sequence from genomic DNA isolated from HEK293 cells by PCR. Using the codon-optimization methods described above, the isolated FXN was modified by site-directed mutagenesis (Stratgene) to reduce the CpG content. The resulting modified amplification product was gel purified and sequenced by methods known in the art.

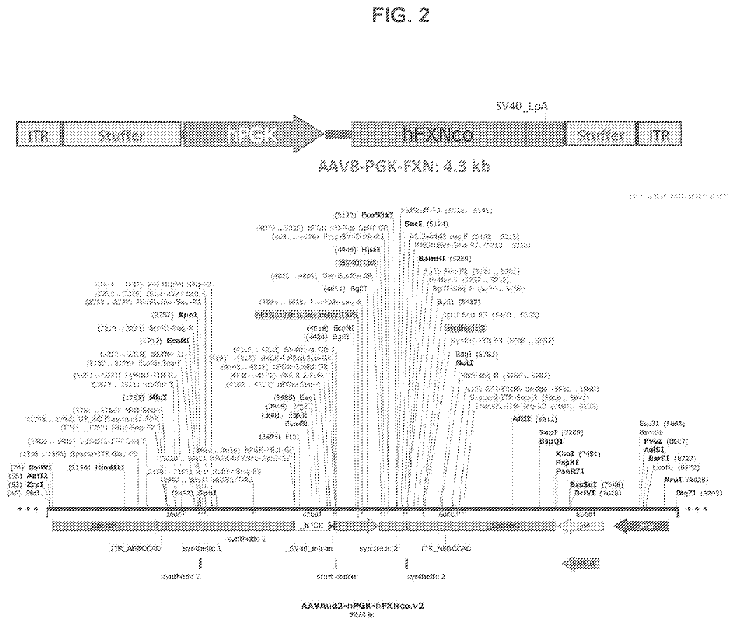

A pseudotyped adeno-associated virus (AAV) 2/8 (AAV 2/8) circular parental vector was used as the destination vector for the codon optimized human FXN variant 1 (hFXNco). The parental vector contains a nucleic acid molecule having the following components: a first spacer (SS1), a first AAV2 inverted terminal repeat (ITR1), a synthetic DNA stuffer, a human phosphoglycerate kinase (PGK) promoter, a simian virus 40 (SV40) intron, a late SV40 polyadenylation site (pA), a second synthetic DNA stuffer, a second AAV2 ITR (ITR2), a prokaryotic origin of replication, a second spacer (SS2), and a kanamycin antibiotic selection gene (Kan^{R}) (FIG. 2). The parental vector also contains a cloning site containing a PmeI restriction endonuclease recognition site 5′-to the first AAV2 ITR1; and a cloning site containing SwaI restriction endonuclease recognition site 3′-to the second AAV2 ITR2. The hFXNco was cloned into the parental vector and the resulting vector included the nucleic acid components operably linked in a 5′-to-3′ direction as: SS1-AAV2 ITR1-PGK-SV40 inron-SV40 LpA-AAV2 ITR2-SS2-oriC-Kan^{R} as mediated by a ligation by T4 DNA ligase at 16° C. for 1 hour. The resulting vector was sequence verified. This above-referenced AAV2/8-PGK-FXN construct represents Version 2 (V2), with an original version (V1) created lacking the synthetic stuffer DNA and having a shorter length between ITR1 and ITR2 of 1.5 Kb.

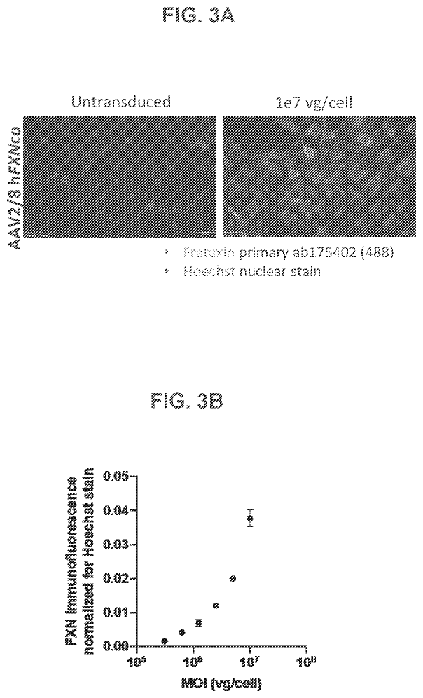

A proof-of-principle experiment was conducted using a cell line derived from mouse skeletal muscle cells (C2C12). In result, the vector preparation yielded robust frataxin expression in transduced C2C12 cells, as demonstrated by immunoflorescence (FIGS. 3A and 3B).

### Friedreich Ataxia Mouse Model

Mice homozygous for a conditional allele of FXN (FXN^{L3/L3}) were crossed with mice heterozygous for the deletion of FXN exon 4 (FXN^{Δ/+}) that carried a muscle-specific Cre recombinase transgene under the control of the muscle creatine kinase (MCK) promoter to induce striated muscle-restricted exon 4 deletion (e.g., see Puccio et al., Nat. Genet. 27(2) (2001): 181-186). Canonically, mutants begin to lose weight at approximately 7 weeks, progressively develop signs of fatigue, and die at 76±10 days.

Using the MCK knockout (KO) mouse as a model for Friedreich ataxia, the safety and efficacy of codon-optimized human frataxin constructs was demonstrated, as described in Example 2.

### Immunoblot Analysis

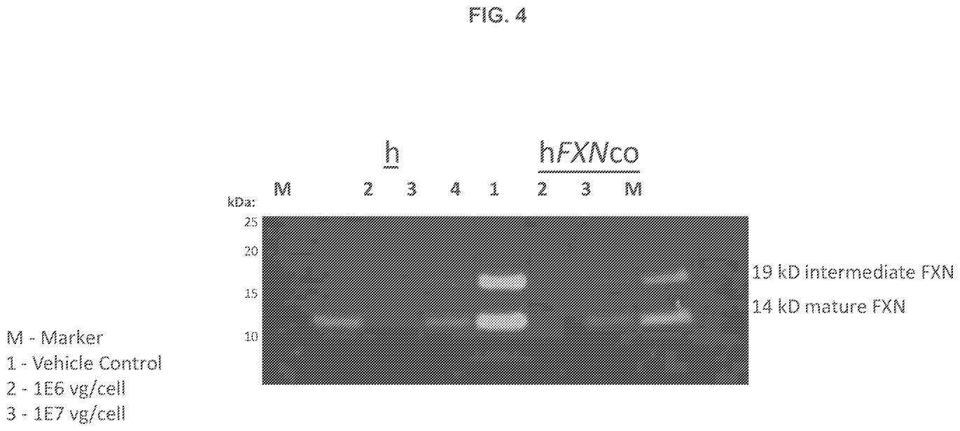

Western blot analysis was performed on whole cell extracts prepared with radioimmunoprecipitation assay (RIPA) buffer. Proteins were separated on 4-15% polyacrylamide gradient-sodium dodecyl sulfate (SDS) gels (Bio-Rad) and transferred onto a Nitrocellulose membrane (Invitrogen). Western blots were visualized with Enhanced Chemiluminescence (Perkin-Elmer). Primary antibodies were used against frataxin (Invitrogen #45-6300) and secondary antibodies were Licor IR Dye 800 CW Donkey anti-mouse. In result, a pseudotyped AAV2/8 viral vector encoding hFXNco effectively expressed the mature isoform of FXN (FIG. 4).

### Example 2. Effectiveness of Codon-Optimized Human FXN on Heart Phenotypes and Mortality in a Model of Friedreich Ataxia

This Example describes the safety and efficacy of a codon optimized FXN gene, including human and murine genes thereof, for example, in a murine model of Friedreich ataxia, for the amelioration of heart phenotypes and early mortality related to Friedreich ataxia.

Materials and methods

Materials and Methods are described in Example 1. This example tested AAV2/8-PGK-FXN version 2 (V2).

### Results

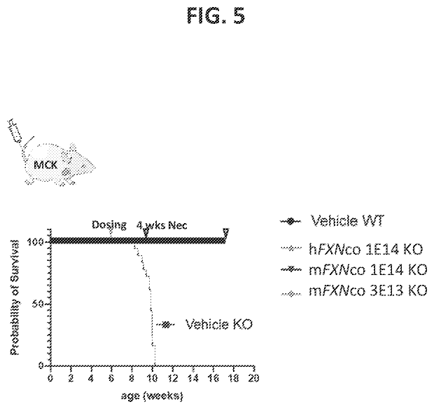

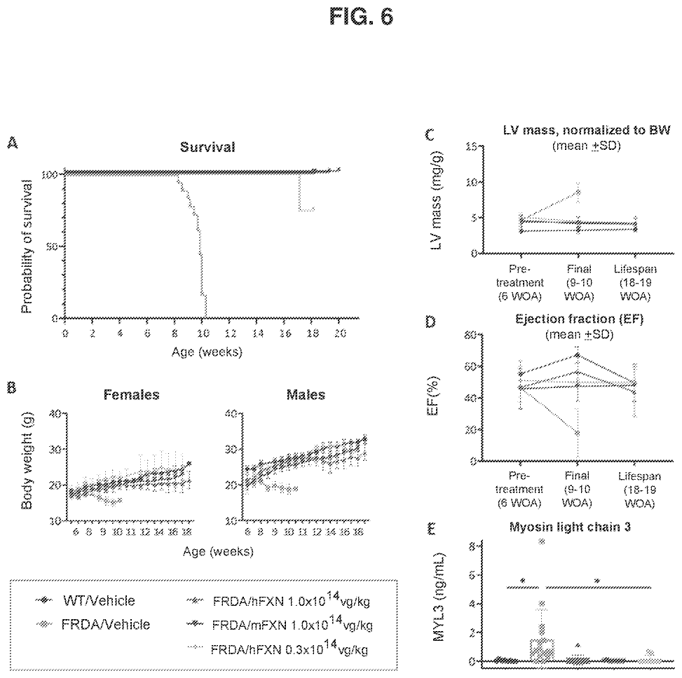

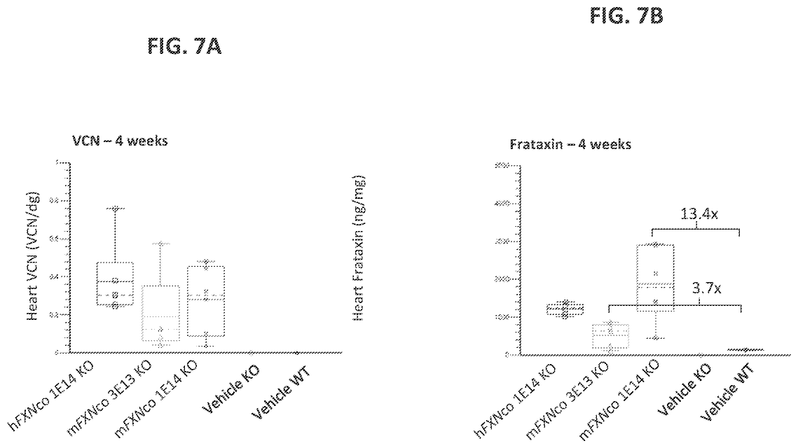

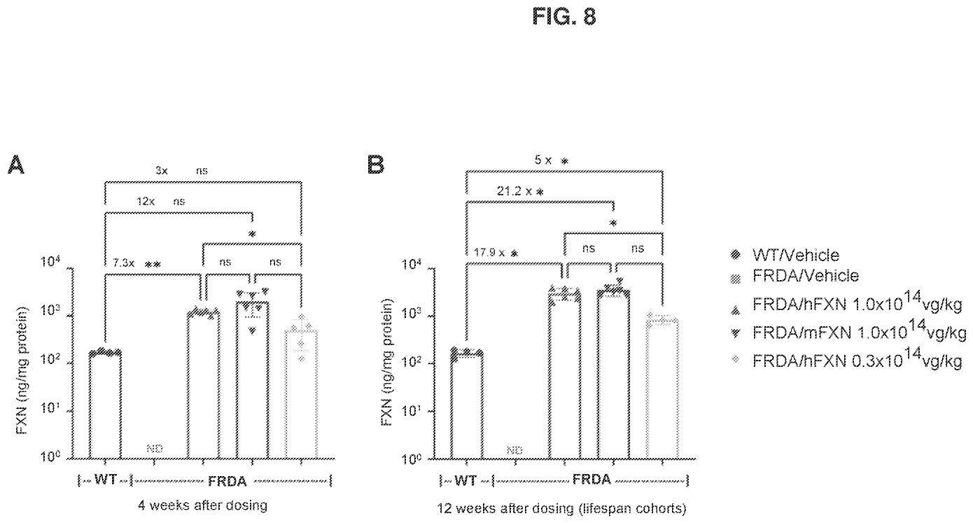

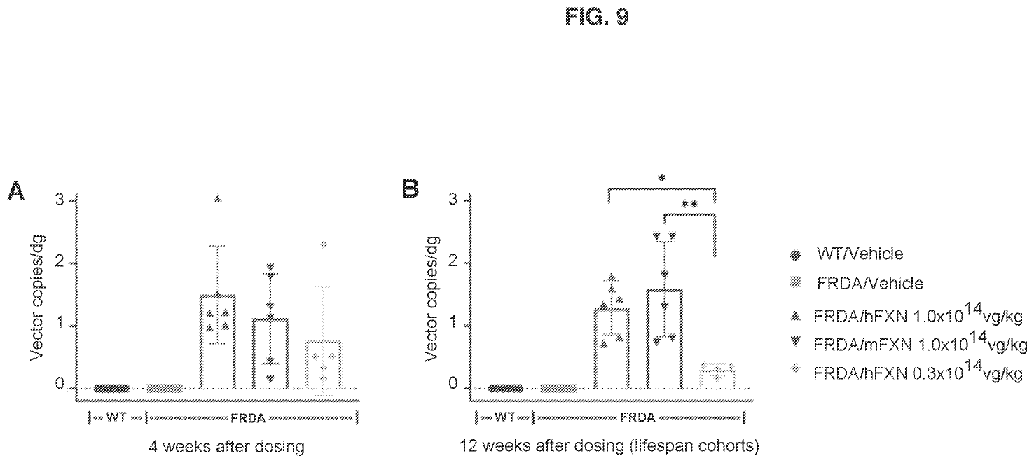

FIGS. 5 and 6A show the probability of survival of FRDA MCK KO mice intravenously administered 3×10¹³ vg/kg or 1×10¹⁴ vg/kg of an exemplary AAV2/8 that encodes a human PGK promoter to drive the expression of hFXNco (hFXN) or the murine equivalent thereof (mFXN); or vehicle controls. Four weeks (wks) after dosing, vehicle KO mice exhibited a sharp decline in the probability of survival, whereas mice administered the hFXN or mFXN survived equivalently to vehicle WT controls. Additionally, in KO mice administered hFXN or mFXN, significant rescue was observed in female and male body weight (FIG. 6B), heart weight normalized by body weight (FIG. 6C) and in ejection fraction (FIG. 6D), as compared to vehicle KO mice. FIGS. 7A and 7B show the level of frataxin expression in biopsies of the heart taken from mice of the same experiment, as measured by a western blot and quantified by the vector copy number (VCN; FIG. 7A) or frataxin protein level (FIG. 7B), respectively. In the heart, no difference in VCN was observed between dose levels at 4 weeks post intravenous administration. As shown in FIG. 7B, a 3.7- and 13.4-fold increase in heart frataxin was observed in KO mice treated with mFXN, as compared to vehicle-treated WT mice (WT mean ˜136 ng/mg), in connection with the read-out shown in FIG. 5. Furthermore, a 3.7- and 23-fold increase in heart frataxin was observed in KO mice treated with hFXN or mFXN, as compared to vehicle-treated WT mice, in connection with the subsequent read-out shown in FIG. 6A. FIG. 8 shows the level of frataxin protein in heart tissue, as measured by enzyme linked immunosorbent assay (ELISA), at 4 weeks and 12 weeks after administration of AAV2/8-PGK-FXN. As shown in FIG. 8, at 4 weeks post administration there was a 3- and 13-fold increase in heart frataxin observed in KO mice treated with mFXN, as compared to vehicle-treated WT mice, and a 7.3-fold increase in heart frataxin observed in KO mice treated with hFXN, as compared to vehicle-treated WT mice. As shown in FIG. 8, at 12 weeks post administration there was a 5- and 21.2-fold increase in heart frataxin observed in KO mice treated with mFXN, as compared to vehicle-treated WT mice, and a 17.9-fold increase in heart frataxin observed in KO mice treated with hFXN, as compared to vehicle-treated WT mice. FIG. 9 shows the number of vector copies per diploid genome detected in the heart tissue, as measured by qPCR, at 4 and 12 weeks after dosing. Vector DNA was detected in the heart tissue of all AAV2/8-PGK-FXN treated KO mice at 4 weeks and 12 weeks after dosing, but not in the vehicle treated mice at either time point. As shown in FIG. 9, at 12 weeks after dosing, significantly more vector copies per genome were detected in the hF×N or 1×10¹⁴ vg/kg mFXN treated mice, as compared to the 3×10¹³ vg/kg mFXN treated mice.

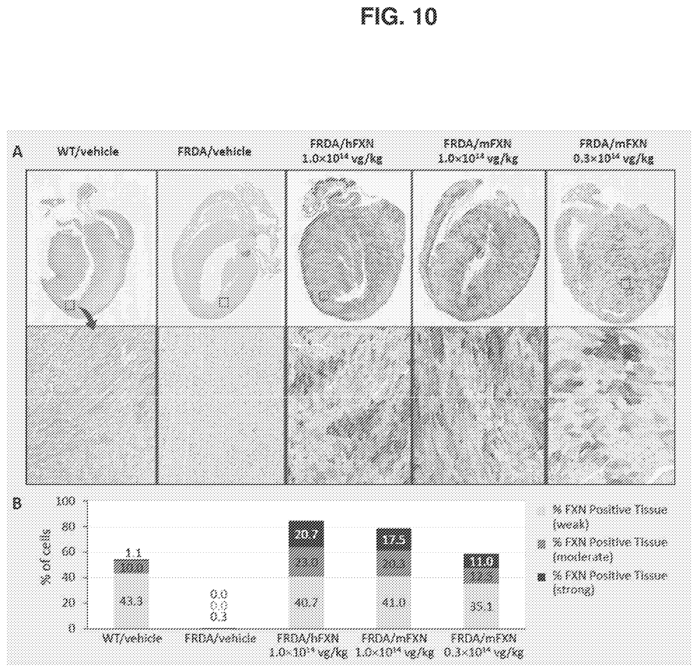

Immunohistochemistry of frataxin expression in heart tissue is shown in FIG. 10A. The percentage of cells exhibiting frataxin expression was similar in the KO mice administered 3×10¹³ vg/kg of an AAV2/8 plasmid encoding mFXN (58.6%) but increased in the KO mice administered 1×10¹⁴ vg/kg of an AAV2/8 plasmid encoding hFXN or mFXN (78-85%), as compared to WT mice (54.4%) (FIG. 10B).

Western blot analysis revealed expression of the mature isoform of frataxin in tissues harvested from mice treated with hFXN or mFXN. Additionally, a myocardial damage marker, myosin light chain, was reduced in the serum of mice treated with hFXN or mFXN (FIG. 6E) and no apparent toxicity or fibrosis was observed in the hearts of treated mice four weeks after dosing.

Taken together, these results demonstrate that an exemplary AAV2/8 plasmid encoding a hFXNco rescued mortality and elicited significant expression of frataxin in the heart in a murine model of Friedreich ataxia.

### Example 3. Use of a Codon-Optimized FXN Gene for the Treatment of Friedreich Ataxia

A gene encoding frataxin can be codon-optimized using the procedures described herein (e.g., as described in Example 1, above). The gene may be codon-optimized with the goals to diminish CpG content and/or homopolymer content in the mRNA transcript or accommodating codon bias, for instance, by introducing codon substitutions into the optimized FXN gene sequence as described in Example 1, above. For example, the final codon-optimized FXN gene may exhibit at least 95% sequence identity to an endogenous RNA molecule encoding the frataxin variant 1 (SEQ ID NO: 5). For example, the final codon-optimized FXN gene may exhibit at least 96%, 97%, 98%, or 99% sequence identity to an endogenous RNA molecule encoding the frataxin variant 1 (SEQ ID NO: 5). In another example, the final codon-optimized gene may have a nucleic acid sequence that is identical to the nucleic sequence of SEQ ID NO: 1.

The gene can subsequently be incorporated into a plasmid, such as a viral vector, and administered to a patient suffering from Friedreich ataxia. For instance, a patient suffering from Friedreich ataxia, a disorder characterized by mutations in the FXN gene, can be administered a viral vector containing a codon-optimized FXN gene under the control of a suitable promoter for expression in a human cell, such as a human muscle cell. For instance, an AAV vector, such as a pseudotyped AAV2/8 vector, can be generated that incorporates the codon-optimized FXN gene between the 5′ and 3′ inverted terminal repeats of the vector, and the gene may be placed under control of a muscle-specific promoter, such as the PGK promoter. The AAV vector can be administered to the subject systemically.

A practitioner of skill in the art can monitor the expression of the codon-optimized FXN gene by a variety of methods. For instance, one of skill in the art can transfect cultured cells, such as C2C12 cells, with the codon-optimized gene in order to model the expression of the codon-optimized gene in the muscles of a patient. Expression of the encoded protein can subsequently be monitored using, for example, an expression assay described herein, such as qPCR, RNA-Seq, ELISA, or an immunoblot procedure. Based on the data obtained from the gene expression assay, further iterations of the codon optimization procedure can be performed, for instance, so as to further diminish CpG content and homopolymer content in the mRNA transcript. Candidate gene sequences with optimal expression patterns in vitro can subsequently be prepared for incorporation into a suitable vector and administration to a mammalian subject, such as an animal model of Friedreich ataxia, or a human patient.

### Example 4. Treatment of Friedreich Ataxia in Human Patients by Administration of Codon-Optimized Human FXN

Using the compositions and methods of the disclosure, a patient (e.g., 3 years of age to 17 years of age) having Friedreich ataxia may be administered a pseudotyped AAV2/8 vector including a nucleic acid sequence encoding a codon-optimized human FXN variant 1 gene operably linked to a PGK promotor, for example, the AAV vector that has a nucleic acid of SEQ ID NO: 4.

Upon administering the pseudotyped AAV2/8 vector including a nucleic acid sequence encoding a codon-optimized human FXN variant 1 gene to the patient, the patient displays a change in whole blood frataxin levels. For example, the patient displays the change in whole blood frataxin levels by about 12 weeks after administration of the pseudotyped AAV2/8 vector including a nucleic acid sequence encoding a codon-optimized human FXN variant 1 to the patient. In addition to, or alternatively, for example, upon administering the pseudotyped AAV2/8 vector including a nucleic acid sequence encoding a codon-optimized human FXN variant 1 gene to the patient, the patient displays a reduction in Total Friedreich Ataxia Rating Scale (FARS) Score. For example, the patient displays the reduction in Total FARS Score by about 12 weeks after administration of the pseudotyped AAV2/8 vector including a nucleic acid sequence encoding a codon-optimized human frataxin variant 1 to the patient.

### Example 5. Use of a Nucleic Acid Molecule with a Short ITR1-to-ITR2 Length and an FXN Gene for the Treatment of Friedreich Ataxia

A gene encoding frataxin can be cloned into a plasmid (e.g., a viral vector) using the procedures described herein (e.g., as described in Example 1, above). The parent plasmid can contain a short ITR1-to-ITR2 length, including the payload, for example about 3.7 Kb to about 4.3 Kb (e.g., about 3.8 Kb to about 4.2 Kb or about 3.9 Kb to about 4.1 Kb). In addition to, or alternatively, the length of the nucleic acids between and including ITR1 and ITR2 may be about 3.9 Kb to about 4.7 Kb (e.g., about 4.0 Kb to about 4.6 Kb, about 4.1 Kb to about 4.5 Kb, about 4.2 Kb to about 4.4 Kb, or about 4.3 Kb). The parent plasmid can subsequently be administered to a patient suffering from Friedreich ataxia. For instance, a patient suffering from Friedreich ataxia, a disorder characterized by mutations in the FXN gene, can be administered a viral vector containing a plasmid including a nucleic acid molecule that includes an ITR1-FXN-ITR2, wherein the ITR1-FXN-ITR2 together are about 3.7 Kb to about 4.3 Kb in length. The FXN gene may under the control of a suitable promoter for expression in a human cell, such as a human muscle cell. For instance, an AAV vector, such as a pseudotyped AAV2/8 vector, can be generated that incorporates the codon-optimized FXN gene between the 5′ and 3′ inverted terminal repeats of the vector, and the gene may be placed under control of a muscle-specific promoter, such as the PGK promoter. The AAV vector can be administered to the subject systemically. The gene may be codon-optimized with the goals to diminish CpG content and/or homopolymer content in the mRNA transcript or accommodating codon bias, for instance, by introducing codon substitutions into the optimized FXN gene sequence as described in Example 1, above. In another example, the final codon-optimized gene may have a nucleic acid sequence that is identical to the nucleic sequence of SEQ ID NO: 1.

A practitioner of skill in the art can monitor the expression of the codon-optimized FXN gene by a variety of methods. For instance, one of skill in the art can transfect cultured cells, such as C2C12 cells, with the codon-optimized gene in order to model the expression of the codon-optimized gene in the muscles of a patient. Expression of the encoded protein can subsequently be monitored using, for example, an expression assay described herein, such as qPCR, RNA-Seq, ELISA, or an immunoblot procedure. Based on the data obtained from the gene expression assay, further iterations of the codon optimization procedure can be performed, for instance, so as to further diminish CpG content and homopolymer content in the mRNA transcript. Candidate gene sequences with optimal expression patterns in vitro can subsequently be prepared for incorporation into a suitable vector and administration to a mammalian subject, such as an animal model of Friedreich ataxia, or a human patient.

### Example 6. Treatment of Friedreich Ataxia in Human Patients by Administration of a Nucleic Acid Molecule with Short ITR1-to-ITR2 Length and an FXN Gene

Using the compositions and methods of the disclosure, a patient (e.g., 3 years of age to 17 years of age) having Friedreich ataxia may be administered a pseudotyped AAV2/8 vector including a nucleic acid sequence encoding frataxin, wherein the AAV2/8 includes an ITR1 and ITR2 flanking the FXN gene and wherein the ITR1-FXN-ITR2 together are about 3.7 Kb to about 4.3 Kb (e.g., about 3.8 Kb to about 4.2 Kb or about 3.9 Kb to about 4.1 Kb) in length. In addition to, or alternatively, the length of the nucleic acids between and including ITR1 and ITR2 may be about 3.9 Kb to about 4.7 Kb (e.g., about 4.0 Kb to about 4.6 Kb, about 4.1 Kb to about 4.5 Kb, about 4.2 Kb to about 4.4 Kb, or about 4.3 Kb). The FXN gene may under the control of a suitable promoter for expression in a human cell, such as a human muscle cell. For instance, the gene may be placed under control of a muscle-specific promoter, such as the PGK promoter. The gene may be codon-optimized with the goals to diminish CpG content and/or homopolymer content in the mRNA transcript or accommodating codon bias, for instance, by introducing codon substitutions into the optimized FXN gene sequence as described in Example 1, above. In another example, the final codon-optimized gene may have a nucleic acid sequence that is identical to the nucleic sequence of SEQ ID NO: 1.

Upon administering the pseudotyped AAV2/8 vector including an ITR1-FXN-ITR2 length that is about 3.7 Kb to about 4.3 Kb to the patient, the patient displays a change in whole blood frataxin levels. For example, the patient displays the change in whole blood frataxin levels by about 12 weeks after administration of the pseudotyped AAV2/8 vector including an ITR1-FXN-ITR2 length that is about 3.7 Kb to about 4.3 Kb to the patient. In addition to, or alternatively, for example, upon administering the pseudotyped AAV2/8 vector including an ITR1-FXN-ITR2 length that is about 3.7 Kb to about 4.3 Kb to the patient, the patient displays a reduction in Total Friedreich Ataxia Rating Scale (FARS) Score. For example, the patient displays the reduction in Total FARS Score by about 12 weeks after administration of the pseudotyped AAV2/8 vector including an ITR1-FXN-ITR2 length that is about 3.7 Kb to about 4.3 Kb to the patient.

### Example 7. A Comparison of the Expression of Codon-Optimized Human FXN in a Pseudotyped AAV2/8 Vector with ITR1-FXN-ITR2 Length of 1.5 Kb and 4.3 Kb

An original version (V1) of an exemplary pseudotyped AAV 2/8 vector that encodes a human PGK promoter to drive the expression of hFXNco was generated as described in Example 1, above. The AAV 2/8 vector contains a nucleic acid molecule having the following components: a first spacer (SS1), a first AAV2 inverted terminal repeat (ITR1), a human phosphoglycerate kinase (PGK) promoter, a simian virus 40 (SV40) intron, a codon-optimized human FXN gene (hFXNco) a late SV40 polyadenylation site (pA), a second AAV2 ITR (ITR2), a prokaryotic origin of replication, a second spacer (SS2), and a kanamycin antibiotic selection gene (Kan^{R}).

A second version (V2) of an exemplary pseudotyped AAV 2/8 vector that encodes a human PGK promoter to drive the expression of hFXNco was generated as described in Example 1, above. The AAV 2/8 vector contains a nucleic acid molecule having the following components: a first spacer (SS1), a first AAV2 inverted terminal repeat (ITR1), a synthetic DNA stuffer, a human phosphoglycerate kinase (PGK) promoter, a simian virus 40 (SV40) intron, a codon-optimized human FXN gene (hFXNco) a late SV40 polyadenylation site (pA), a second synthetic DNA stuffer, a second AAV2 ITR (ITR2), a prokaryotic origin of replication, a second spacer (SS2), and a kanamycin antibiotic selection gene (Kan^{R}) (FIG. 2). This second version (V2) of AAV2/8-PGK-FXN differs from V1 by the presence of two synthetic DNA stuffers to achieve an optimal sequence length between ITR1 and ITR2 of 4.3 Kb. A comparison of AAV2/8-PGK-FXN V1 and V2 is summarized in Table 3 below.

TABLE 3
Comparison of Genetic Components
of AAV2/8-PGK-FXN V1 and V2

AAV2/8-PGK-FXN

| Genetic Component | V1 | V2 |
|---|---|---|
| ITR1 | Yes | Yes |
| DNA stuffer 1 | No | Yes |
| PGK promoter | Yes | Yes |
| SV40 | Yes | Yes |
| hFXNco | Yes | Yes |
| pA | Yes | Yes |
| DNA stuffer 2 | No | Yes |
| ITR2 | Yes | Yes |
| Total Nucleotide Length | 1.5 Kb | 4.3 Kb |
| Between ITR1 and ITR2 | | |

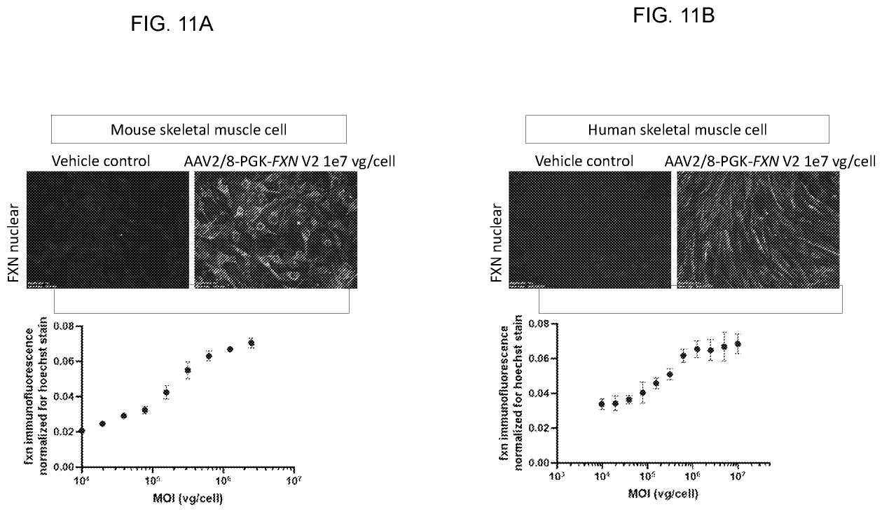

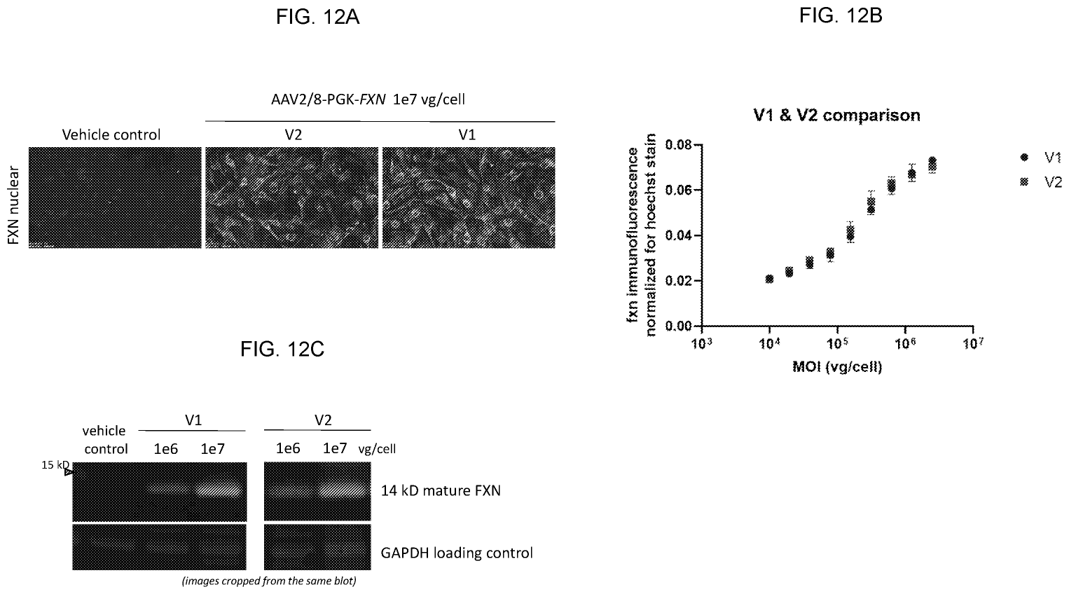

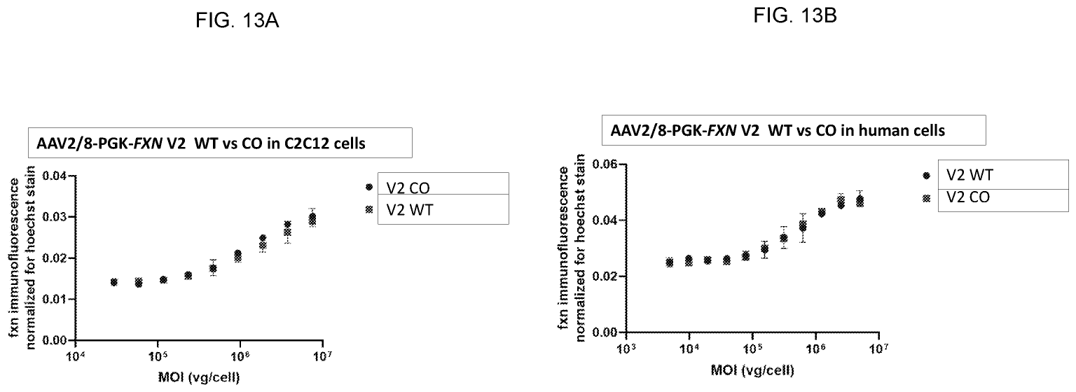

To test expression of FXN in AAV2/8-PGK-FXN V2, mouse and human muscle cell lines were transduced with AAV2/8-PGK-FXN V2 at increasing doses. Dose-dependent FXN expression was observed following transduction with AAV2/8-PGK-FXN V2 in mouse (FIG. 11A) and human (FIG. 11B) muscle cell lines. When compared with AAV2/8-PGK-FXN V1, V2 expressed at the same level (FIG. 12A and FIG. 12B) and correctly processed to yield 14 kDa mature FXN (FIG. 12C). Codon-optimized human FXN showed similar levels of expression as WT human FXN in AAV2/8-PGK-FXN V2 when C2C12 or AB1079 cells were treated (FIG. 13).

### Example 8. Effectiveness of Codon-Optimized Human FXN on Cardiac Phenotypes and Mortality in a Mouse Model of Friedreich Ataxia

This Example describes the safety and efficacy of a codon optimized FXN (hFXNco) gene in a murine model of Friedreich ataxia, for the amelioration of heart phenotypes and early mortality related to Friedreich ataxia.

### Materials and Methods

Mice were administered a single dose of version 1 (V1 positive control) or version 2 (V2) of an exemplary AAV8 vector that encodes a human PGK promoter to drive the expression of hFXNco (AAV8-PGK-FXN) intravenously at 6 weeks of age to evaluate the efficacy and toxicology of a codon optimized FXN gene in a Friedreich's Ataxia mouse model (described in Example 1, above). Study parameters for each treatment group are described in Table 4 below.

TABLE 4
Study parameters for evaluating AAV8-PGK-FXN V2

| | | | | | | Necropsy Time- | |
|---|---|---|---|---|---|---|---|
| | | | | Dose | Dosing | point (post- | |
| Group | N | Genotype | Compounds | (vg/kg) | Route | injection) | |
| 1 | 5F/5M | Wildtype | Vehicle | — | 1x IV | 4 | weeks |
| | | (C57BL/6J) | | | | | |
| 2 | 5F/5M | Wildtype | Vehicle | — | 1x IV | 12 | weeks |
| | | (C57BL/6J) | | | | | |
| 3 | 5F/5M | MCK-FXN-KO | Vehicle | — | 1x IV | 4 | weeks |
| 4 | 5F/5M | MCK-FXN-KO | V2 | 1E12 | 1x IV | 4 | weeks |
| 5 | 5F/5M | MCK-FXN-KO | V2 | 1E12 | 1x IV | 12 | weeks |
| 6 | 5F/5M | MCK-FXN-KO | V2 | 3E12 | 1x IV | 4 | weeks |
| 7 | 5F/5M | MCK-FXN-KO | V2 | 3E12 | 1x IV | 12 | weeks |
| 8 | 5F/5M | MCK-FXN-KO | V2 | 1E13 | 1x IV | 4 | weeks |
| 9 | 5F/5M | MCK-FXN-KO | V2 | 1E13 | 1x IV | 12 | weeks |
| 10 | 5F/5M | MCK-FXN-KO | V2 | 3E13 | 1x IV | 4 | weeks |
| 11 | 5F/5M | MCK-FXN-KO | V2 | 3E13 | 1x IV | 12 | weeks |
| 12 | 5F/5M | MCK-FXN-KO | V2 | 1E14 | 1x IV | 4 | weeks |
| 13 | 5F/5M | MCK-FXN-KO | V2 | 1E14 | 1x IV | 12 | weeks |
| 14 | 5F/5M | MCK-FXN-KO | V1 - Original | 3E13 | 1x IV | 12 | weeks |

### Results

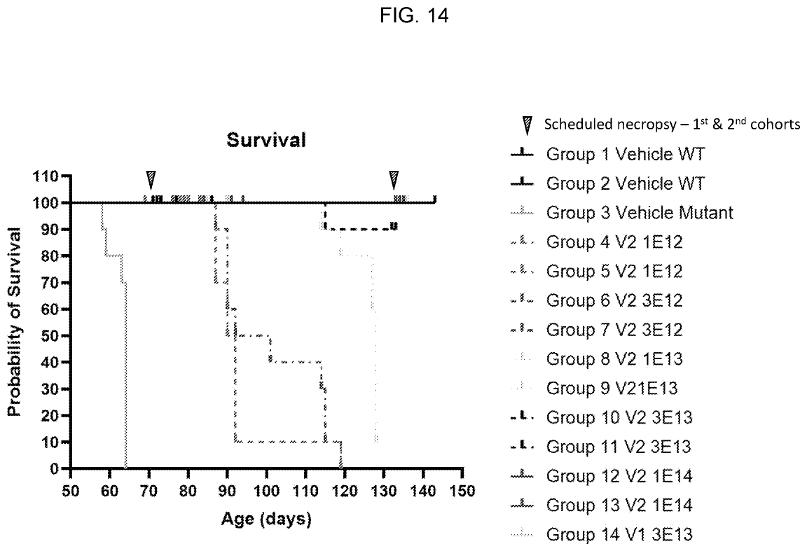

Survival studies were carried out to assess the effect of hFXNco on mortality. Mice were euthanized if the animals displayed any of the following conditions prior to scheduled necropsy: >20% decrease in body weight, demonstrating signs of respiratory distress, unresponsiveness to meaningful stimuli, and/or overall poor body condition. Median age at euthanasia was significantly different between vehicle treated (untransduced control) and all other treatment groups, with dose-dependent rescue of mortality observed from the AAV8-PGK-FXN V1 and V2 treated animals (FIG. 14).

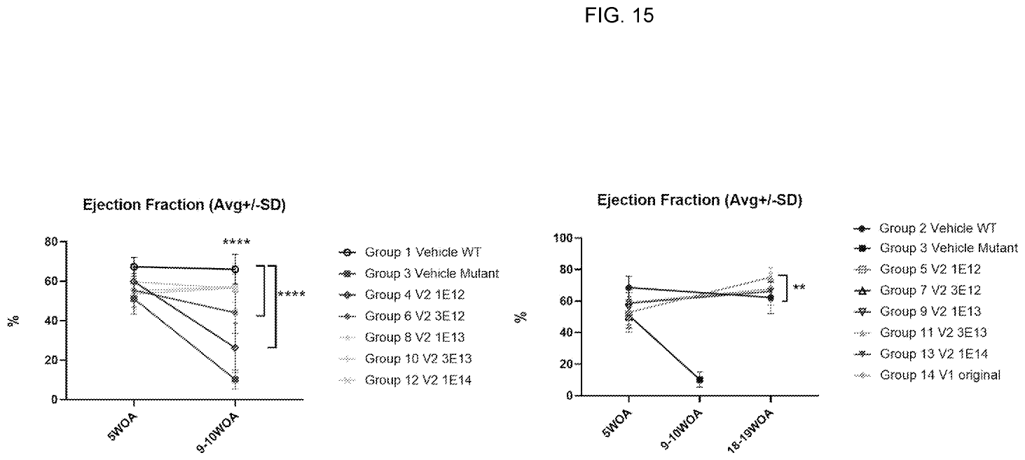

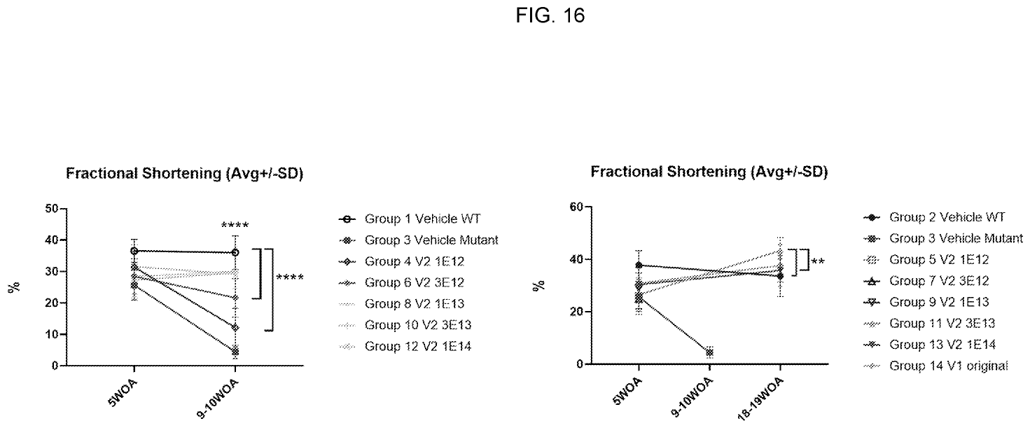

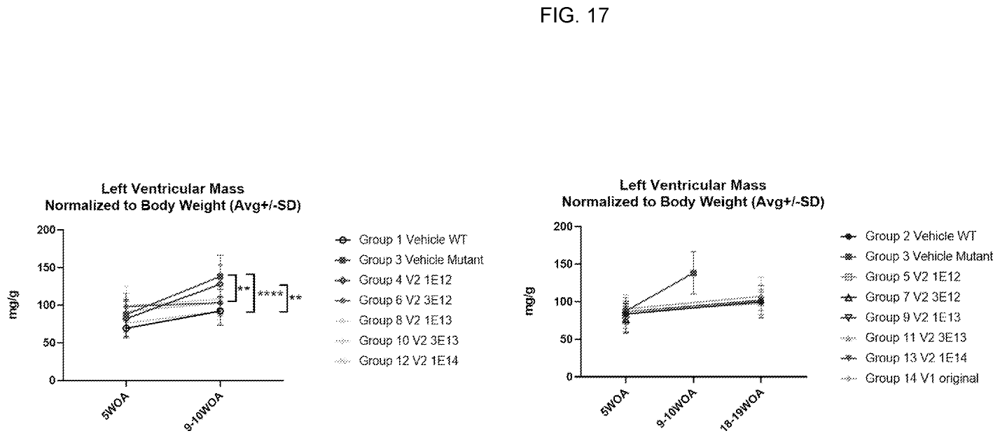

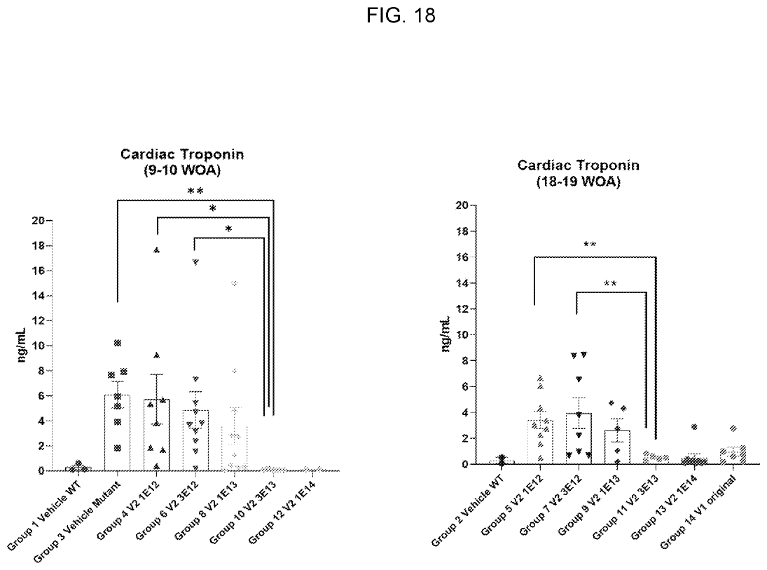

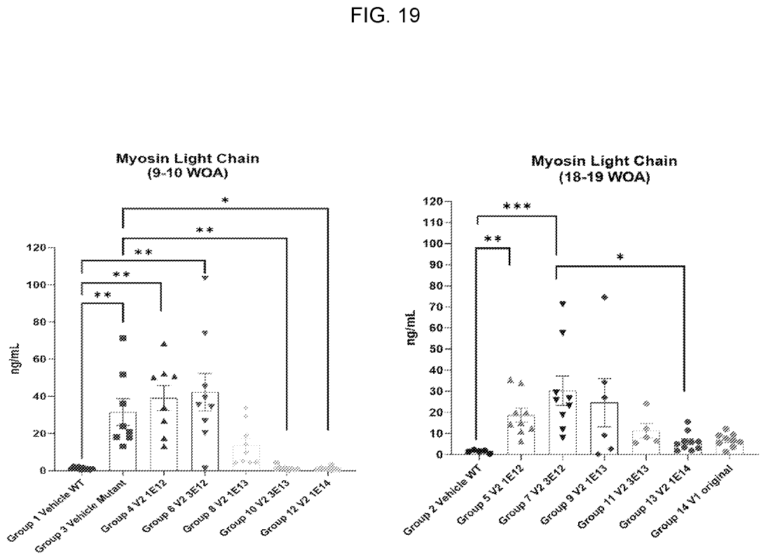

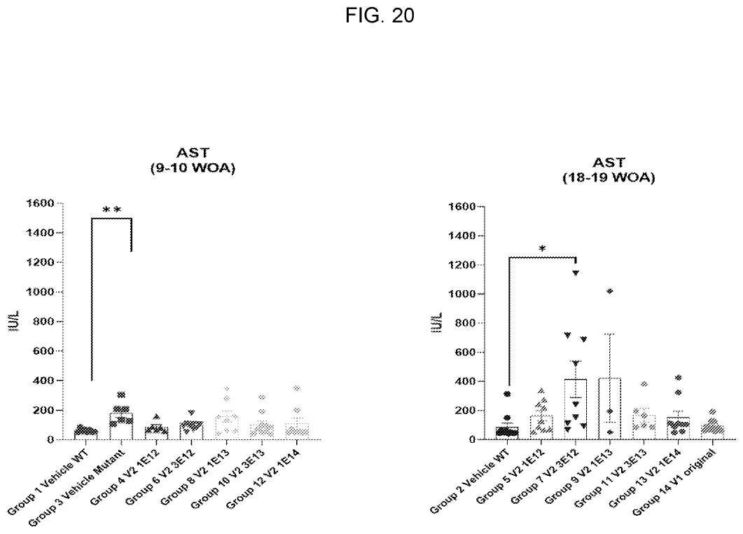

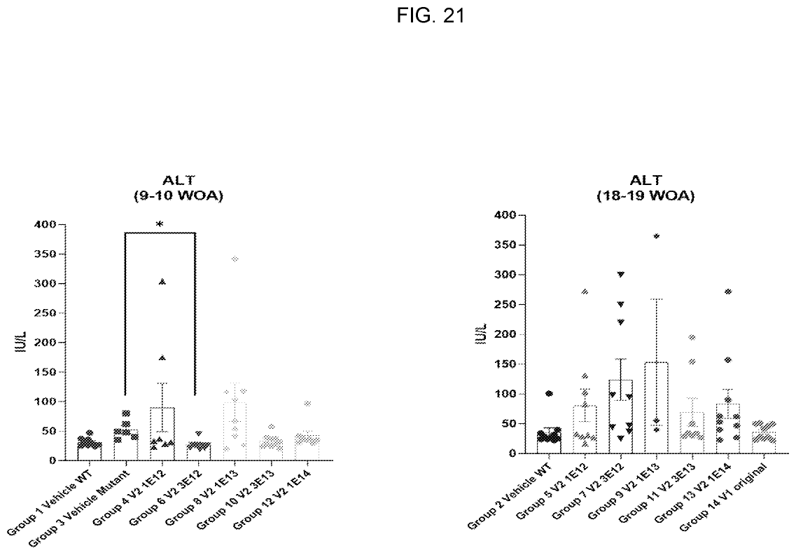

Cardiac function was evaluated using high-frequency ultrasound over the course of the study starting at 6 weeks of age (WOA) and at endpoint, either 9-10 WOA or 18-19 WOA (as detailed in Table 4). The administration of AAV8-PGK-FXN V1 and V2 rescued cardiac function as evidenced by the increases in ejection fraction (FIG. 15) and fractional shortening (FIG. 16) and the decrease in left ventricular mass (FIG. 17). AAV8-PGK-FXN V1 and V2 reduced the levels of cardiac injury markers cardiac troponin (FIG. 18) and Myosin light chain (FIG. 19) in the serum, while no significant changes in AST and ALT were observed (FIGS. 20 and 21).

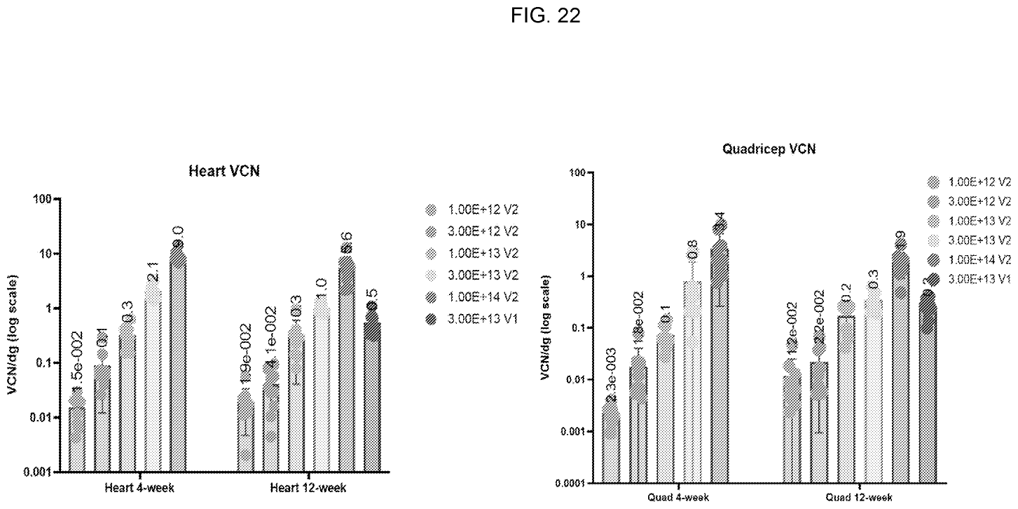

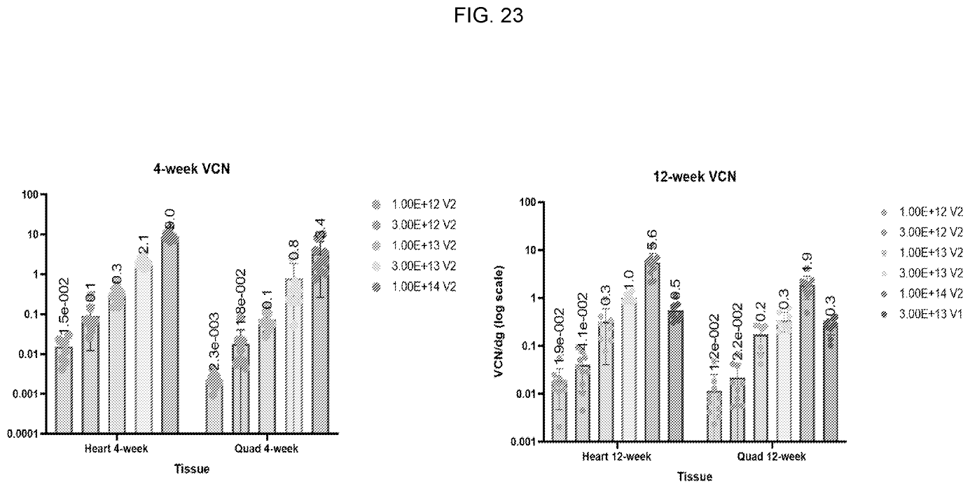

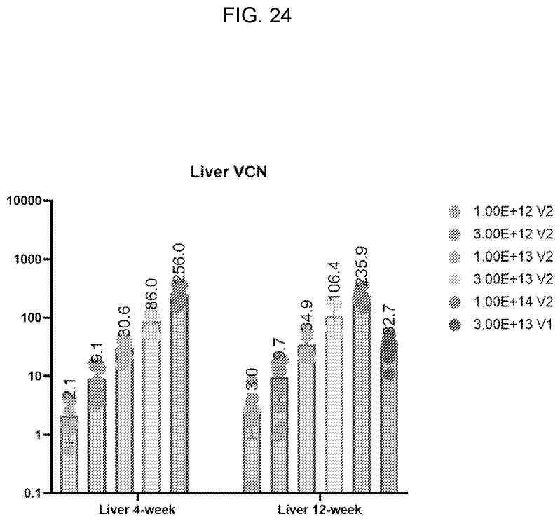

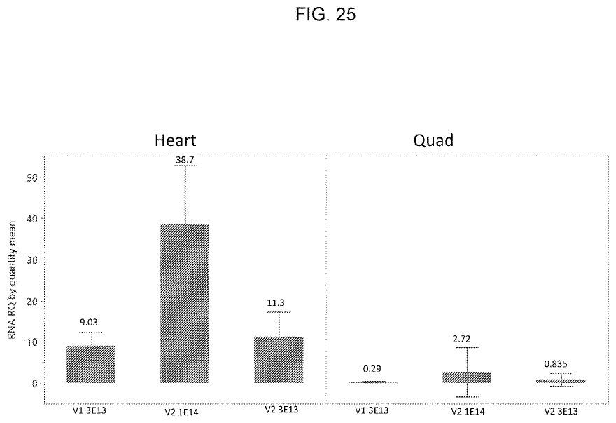

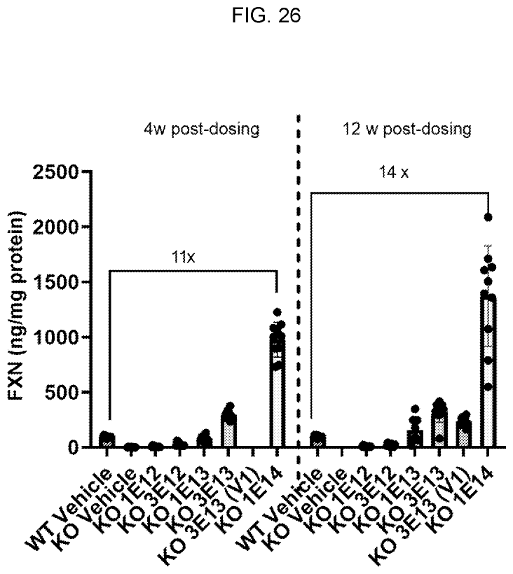

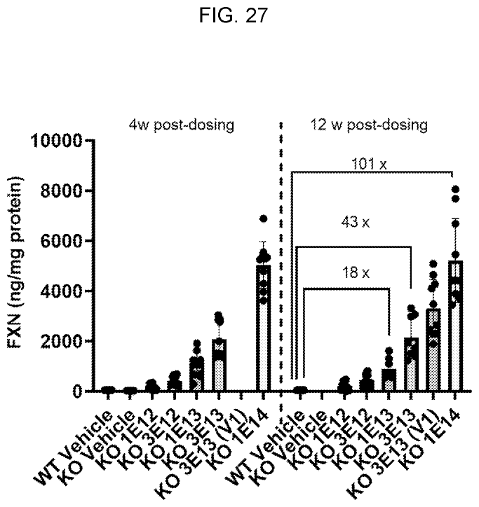

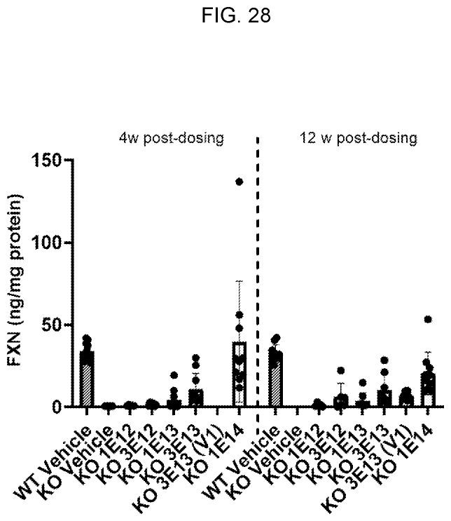

Transduction and expression of AAV8-PGK-FXN V2 were evaluated by measuring vector copy numbers (VON), mRNA, and FXN protein expression in the heart, liver, and quadricep. Dose-dependent VON was detected in the heart, liver, and quad (FIGS. 22-24), with approximately 3-fold lower VON in the quadricep than in the heart for most groups (FIG. 23), indicating better transduction in the heart than the quadricep. FXN mRNA was detected in the heart and quadriceps of mutant mice in a dose-dependent manner for mutant mice treated with AAV8-PGK-FXN V2 (FIG. 25). There was a dose-dependent increase in FXN protein expression in the heart (FIG. 26), liver (FIG. 27), and quadricep (FIG. 28), with AAV8-PGK-FXN V2-driven protein showing much lower expression in the quadricep than in the heart.

### Conclusion

Taken together, these results demonstrate that V2 of an exemplary AAV8 plasmid encoding a hFXNco under control of a human PGK promoter rescued mortality and cardiac function, and elicited significant expression of frataxin in the heart, liver, and quadricep in a murine model of Friedreich ataxia.

### Example 9. Effectiveness of Codon-Optimized Human FXN on Central Nervous System Phenotypes in a Neuron-Specific Mouse Model of Friedreich Ataxia

This Example describes the efficacy of a codon optimized human FXN (hFXNco) gene in a neuron-specific Friedreich ataxia mouse model, for the amelioration of central nervous system (CNS) phenotypes related to Friedreich ataxia.

Materials and Methods Mice were administered a single intracerebroventricular (ICV) or intraparenchymal (IPC) dose of version 1 (V1 positive control) and version 2 (V2) of an exemplary AAV8 vector that encodes a human PGK promoter to drive the expression of hFXNco (AAV8-PGK-FXN) at 8 weeks of age to evaluate the efficacy and toxicology of a codon optimized FXN gene in a neuron-specific Friedreich's Ataxia mouse model.

Mice homozygous for frataxin floxed exon 2 were crossed with mice heterozygous for both PV-Cre knockin and frataxin global KO to induce Fxn^{flox/null}::PV-Cre genotype, which is compound heterozygous at the frataxin locus (floxed exon 2 and global KO on respective homologous chromosomes) and heterozygous for the PV-Cre knockin allele. Fxn^{flox/null}::PV-Cre mice have a Cre-conditional frataxin allele, global knockout frataxin allele, and parvalbumin neuron-specific Cre recombinase knockin allele, creating an early onset ataxia mouse model useful for the study of Friedreich's Ataxia.

Using the Fxn^{flox/null}::PV-Cre knockout (KO) mouse as a model for Friedreich ataxia, the efficacy of codon-optimized human frataxin constructs was demonstrated.

Study parameters for each treatment group are described in Table 5 below.

TABLE 5
Study parameters for evaluating AAV8-PGK-FXN V2

| | | Dose | Dosing | | Dose (total | Necropsy |
|---|---|---|---|---|---|---|
| Group | N | age | Route | Treatment | vg/animal) | (weeks) |
| 1 - WT (B6) | 8F/8M | N/A | N/A | NHS | N/A | 8 |
| 2 - KO | 8F/8M | | | | | |
| | 15F/7M | N/A | N/A | NHS | N/A | |
| 3 - WT (B6) | 8F/8M | 8 weeks | Bilateral ICV | Vehicle | N/A | 11 |
| 4 - KO | 8F/8M | 8 weeks | Bilateral ICV | Vehicle | N/A | 11 |
| 5 - KO | 8F/8M | Postnatal | Bilateral ICV | V2 | 3.00E+11 | 4 |
| | | day 2 | | | | |
| 6 - KO | 8F/8M | 8 weeks | Bilateral ICV | V2 | 6.00E+10 | 11 |
| 7 - KO | 8F/8M | 8 weeks | Bilateral ICV | V2 | 3.00E+11 | 10 |
| 8 - KO | 8F/8M | 8 weeks | Bilateral ICV | V1 | 6.00E+10 | 12 |
| 9 - KO | 6F/6M | 8 weeks | Bilateral DN | V2 | 6.00E+10 | 5 |
| | | | IPC | | | |
| 10 - KO | 6F/6M | 8 weeks | Bilateral DN | V2 | 2.40E+11 | 5 |
| | | | IPC | | | |
| 11 - WT | 4F/4M | Postnatal | Bilateral ICV | Vehicle | N/A | 4 |
| | | day 2 | | | | |
| 12 - KO | 4F/4M | Postnatal | Bilateral ICV | Vehicle | N/A | 4 |
| | | day 2 | | | | |
| 13 - WT (B6) | 4F/4M | 8 weeks | Bilateral DN | Vehicle | N/A | — |
| | | | IPC | | | |
| 14 - KO | 4F/4M | 8 weeks | Bilateral DN | Vehicle | N/A | — |
| | | | IPC | | | |

### Results

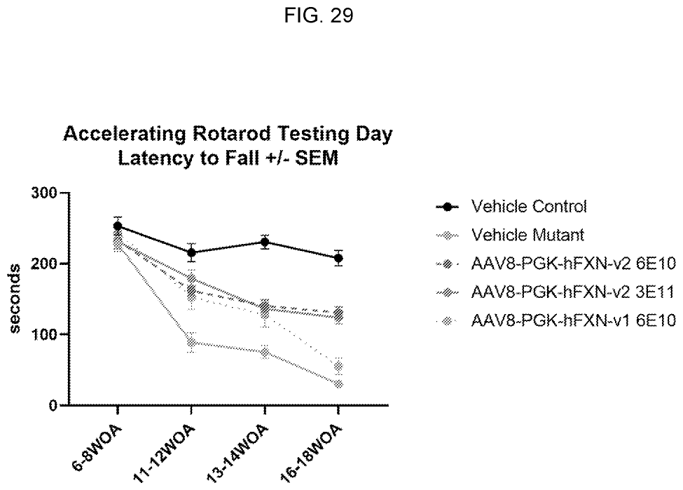

The effect of hFXNco on motor coordination was evaluated using a rotarod performance test. Treatment with AAV8-PGK-FXN V2 significantly improved the time to fall when compared to the vehicle (untransduced control) mutant (FIG. 29).

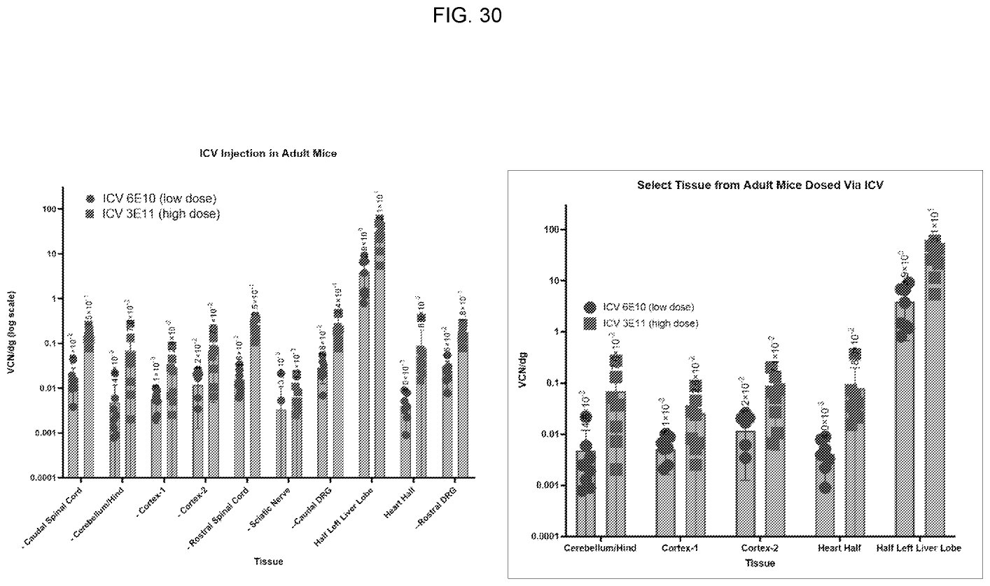

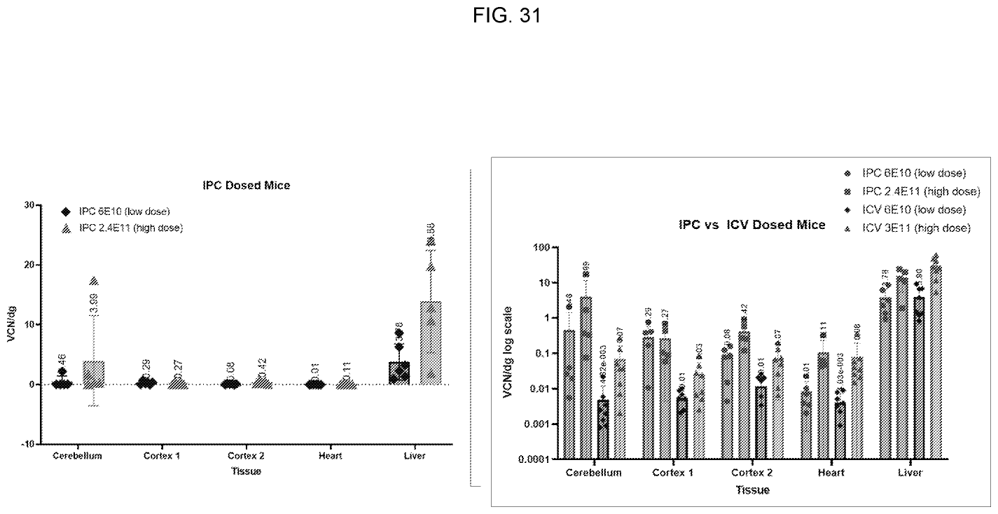

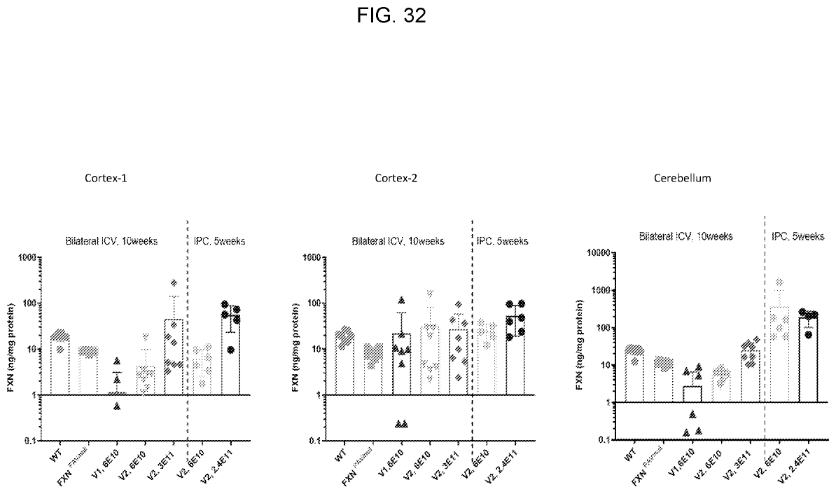

Transduction and expression of AAV8-PGK-FXN V2 were evaluated by measuring vector copy numbers (VON) and FXN protein expression in the ONS tissues. Oose-dependent VON results were observed in animals dosed via ICV at 8-weeks old, with brain VON showing high variability (FIG. 30). IPC-dosed animals showed 100-fold higher VON in the cerebellum and about 10-fold higher VON in the cortex than animals dosed via ICV at 8 weeks old (FIG. 31). FXN protein expression was observed in the cortex and cerebellum for animals dosed with AAV8-PGK-FXN V2 at 8 weeks old, with IPC injection improving AAV8-PGK-FXN V2 delivery to the cerebellum (FIG. 32).

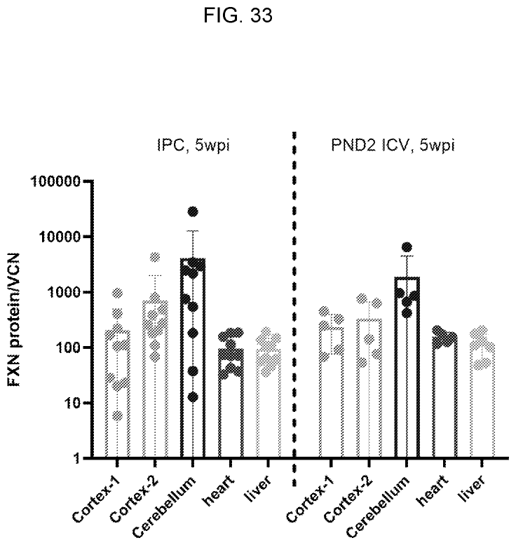

The cerebellum is an important indication in Friedreich ataxia, as the disease causes damage to the cerebellum portion of the brain. After AAV8-PGK-FXN V2 dosing, the cerebellum produced higher amounts of FXN proteins per the same amount of VON than other tissues (FIG. 33), indicating that less AAV8-PGK-FXN V2 is needed for therapeutic benefits in the cerebellum.

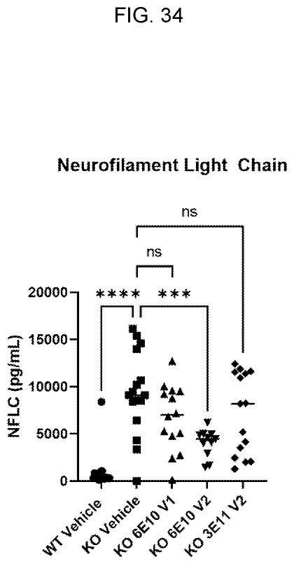

Neurofilament light chain (NFLC) is a neuron-specific cytoskeletal protein that is released into the extracellular fluid following axonal injury and is recognized as an important biomarker in many neurodegenerative diseases. Following treatment with AAV8-PGK-FXN V2, mutant mice were found to have lowered levels of NFLC neural injury marker (FIG. 34).

### Conclusion

Taken together, these results demonstrate that V2 of an exemplary AAV8 plasmid encoding a hFXNco under control of a human PGK promoter rescued CNS phenotypes, and elicited expression of frataxin in CNS tissues in a neuron-specific mouse model of Friedreich ataxia.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A DNA polynucleotide encoding human frataxin (hFXN) or an RNA equivalent thereof, wherein the polynucleotide has a nucleic acid sequence that is at least 95% identical to the nucleic acid sequence of SEQ ID NO: 1.

2. The polynucleotide of claim 1, wherein the polynucleotide has a nucleic acid sequence that is at least 96% identical to the nucleic acid sequence of SEQ ID NO: 1.

3. The polynucleotide of claim 2, wherein the polynucleotide has a nucleic acid sequence that is at least 97% identical to the nucleic acid sequence of SEQ ID NO: 1.

4. The polynucleotide of claim 3, wherein the polynucleotide has a nucleic acid sequence that is at least 98% identical to the nucleic acid sequence of SEQ ID NO: 1.

5. The polynucleotide of claim 4, wherein the polynucleotide has a nucleic acid sequence that is at least 99% identical to the nucleic acid sequence of SEQ ID NO: 1.

6. The polynucleotide of claim 5, wherein the polynucleotide has the nucleic acid sequence of SEQ ID NO: 1.

7. A vector comprising the polynucleotide of any one of claims 1-6, optionally wherein the vector is a plasmid, a DNA vector, an RNA vector, a virion, or a viral vector.

8. The vector of claim 7, wherein the vector is a viral vector.

9. The vector of claim 8, wherein the viral vector is selected from the group consisting of an adeno-associated virus (AAV), an adenovirus, a lentivirus, a retrovirus, a poxvirus, a baculovirus, a herpes simplex virus, a vaccinia virus, and a synthetic virus.

10. The vector of claim 9, wherein the viral vector is an AAV.

11. The vector of claim 10, wherein the AAV comprises capsid proteins from an AAV serotype selected from the group consisting of AAV1, AAV2, AAV3, AAV4, AAV5, AAV6, AAV7, AAV8, AAV9, AAVrh10, and AAVrh74.

12. The vector of claim 10 or 11, wherein the viral vector is a pseudotyped AAV.

13. The vector of claim 12, wherein the pseudotyped AAV is AAV2/8 or AAV2/9, optionally wherein the pseudotyped AAV is AAV2/8.

14. The vector of any one of claims 10-13, wherein the AAV comprises a recombinant capsid protein.

15. The vector of any one of claims 7-14, wherein the polynucleotide is operably linked to a muscle specific promoter, optionally wherein the promoter is positioned 5′ to the polynucleotide.

16. The vector of claim 15, wherein the muscle specific promotor is a phosphoglycerate kinase (PGK) promoter, a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3.

17. The vector of claim 16, wherein the muscle specific promoter is a PGK promoter.

18. The vector of claim 17, wherein the PGK promoter has a nucleic acid sequence that is at least 85% identical to the nucleic acid of sequence of SEQ ID NO: 2.

19. The vector of claim 18, wherein the PGK promoter has a nucleic acid sequence that is at least 90% identical to the nucleic acid of sequence of SEQ ID NO: 2.

20. The vector of claim 19, wherein the PGK promoter has a nucleic acid sequence that is at least 95% identical to the nucleic acid of sequence of SEQ ID NO: 2, optionally wherein the PGK promoter has a nucleic acid sequence that is at least 96%, 97%, 98%, or 99% identical to the nucleic acid sequence of SEQ ID NO: 2.

21. The vector of claim 20, wherein the PGK promoter has the nucleic acid of SEQ ID NO: 2.

22. The vector of any one of claims 7-21, wherein the vector further comprises a polyadenylation site (pA), optionally wherein the pA is positioned 3′ to the polynucleotide.

23. The vector of claim 22, wherein the pA site comprises the simian virus 40 (SV40) late polyadenylation site, the SV40 early polyadenylation site, the human β-globin polyadenylation site, or the bovine growth hormone polyadenylation site.

24. The vector of claim 23, wherein the pA site comprises the SV40 late polyadenylation site.

25. The vector of any one of claims 15-24, wherein the vector further comprises an intron, optionally wherein the intron is positioned 3′ to the promoter and 5′ to the polynucleotide.

26. The vector of claim 25, wherein the intron is an SV40 intron.

27. The vector of any one of claims 10-26, wherein the AAV further comprises two inverted terminal repeats (ITRs), wherein the two ITRs comprise a first ITR (ITR1) and a second ITR (ITR2), wherein ITR1 is positioned 5′ to the polynucleotide and ITR2 is positioned 3′ to the polynucleotide to form a cassette comprising the structure ITR1-hFXN-ITR2.

28. The vector of claim 27, wherein the length of the nucleic acid between ITR1 and ITR2 is from about 3.7 Kb to about 4.3 Kb.

29. The vector of claim 28, wherein the length of the nucleic acid between ITR1 and ITR2 is from about 3.8 Kb to about 4.2 Kb.

30. The vector of claim 29, wherein the length of the nucleic acid between ITR1 and ITR2 is from about 3.9 Kb to about 4.1 Kb.

31. The vector of claim 30, wherein the length of the nucleic acid between ITR1 and ITR2 is about 4.0 Kb.

32. The vector of any one of claims 27-31, wherein the length of the nucleic acid between and including ITR1 and ITR2 is from about 3.9 Kb to about 4.7 Kb.

33. The vector of claim 32, wherein the length of the nucleic acid between and including ITR1 and ITR2 is from about 4.1 Kb to about 4.5 Kb.

34. The vector of claim 33, wherein the length of the nucleic acid between and including ITR1 and ITR2 is about 4.3 Kb.

35. The vector of any one of claims 27-34, wherein the two ITRs are AAV serotype 2 ITRs.

36. A plasmid encoding the viral vector of any one of claims 8-35.

37. The plasmid of claim 36, wherein the plasmid further comprises one or more spacer elements (SS).

38. The plasmid of claim 37, wherein the one or more SS do not comprise an open reading frame that is greater than 100 amino acids in length.

39. The plasmid of claim 37, wherein the one or more SS do not comprise prokaryotic transcription factor binding sites.

40. The plasmid of any one of claims 37-39, wherein the vector comprises two spacer elements: a first spacer element (SS1) and a second spacer element (SS2).

41. The plasmid of claim 40, wherein the SS1 is from about 1.0 Kb to about 5.0 Kb in length.

42. The plasmid of claim 41, wherein the SS1 is from about 2.0 Kb to about 5.0 Kb in length.

43. The plasmid of any one of claims 40-42, wherein the SS2 is from about 1.0 Kb to about 5.0 Kb in length.

44. The plasmid of claim 43, wherein the SS2 is from about 2.0 Kb to about 5.0 Kb in length.

45. The plasmid of claim any one of claims 37-44, wherein the SS is positioned 5′ to ITR1 and/or 3′ to ITR2.

46. The plasmid of any one of claims 40-44, wherein the SS1 is positioned 5′ to ITR1 and the SS2 is positioned 3′ to ITR2.

47. A pharmaceutical composition comprising the composition of any one of claims 1-6, the vector of any one of claims 7-35, or the plasmid of any one of claims 36-46 and a pharmaceutically acceptable carrier, diluent, or excipient.

48. A nucleic acid molecule comprising:
(i) an ITR1;
(ii) a hFXN or an RNA equivalent thereof; and
(iii) an ITR2;
wherein the components are operably linked to each other in a 5′-to-3′ direction as:
ITR1-hFXN-ITR2; and wherein the length of the nucleic acid between ITR1 and ITR2 is from about 3.7 Kb to about 4.3 Kb.

49. The nucleic acid molecule of claim 48, wherein the length of the nucleic acid between ITR1 and ITR2 is from about 3.8 Kb to about 4.2 Kb.

50. The nucleic acid molecule of claim 49, wherein the length of the nucleic acid between ITR1 and ITR2 is from about 3.9 Kb to about 4.1 Kb.

51. The nucleic acid molecule of claim 50, wherein the length of the nucleic acid between ITR1 and ITR2 is about 4.0 Kb.

52. The nucleic acid molecule of any one of claims 48-51, wherein the length of the nucleic acid between and including ITR1 and ITR2 is from about 3.9 Kb to about 4.7 Kb.

53. The nucleic acid molecule of claim 52, wherein the length of the nucleic acid between and including ITR1 and ITR2 is from about 4.1 Kb to about 4.5 Kb.

54. The nucleic acid molecule of claim 53, wherein the length of the nucleic acid between and including ITR1 and ITR2 is about 4.3 Kb.

55. The nucleic acid molecule of any one of claims 48-54, wherein the nucleic acid molecule further comprises:
(iv) a eukaryotic promoter (P_{Euk}),
wherein the components are operably linked to each other in a 5′-to-3′ direction as:
ITR1-P_{Euk}-hFXN-ITR2.

56. The nucleic acid molecule of claim 55, wherein the P_{Euk} is a muscle specific promoter.

57. The nucleic acid molecule of claim 56, wherein the muscle specific promoter is a PGK promoter, a desmin promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3, a cytomegalovirus promoter, or a chicken-β-actin promoter.

58. The nucleic acid molecule of claim 57, wherein the muscle specific promoter is a PGK promoter.

59. The nucleic acid molecule of claim 58, wherein the PGK promoter has a nucleic acid sequence that is at least 85% identical to the nucleic acid of sequence of SEQ ID NO: 2.

60. The nucleic acid molecule of claim 59, wherein the PGK promoter has a nucleic acid sequence that is at least 90% identical to the nucleic acid of sequence of SEQ ID NO: 2.

61. The nucleic acid molecule of claim 60, wherein the PGK promoter has a nucleic acid sequence that is at least 95% identical to the nucleic acid of sequence of SEQ ID NO: 2, optionally wherein the PGK promoter has a nucleic acid sequence that is at least 96%, 97%, 98%, or 99% identical to the nucleic acid sequence of SEQ ID NO: 2.

62. The nucleic acid molecule of claim 61, wherein the PGK promoter has the nucleic acid of SEQ ID NO: 2.

63. The nucleic acid molecule of any one of claims 55-62, wherein the nucleic acid molecule further comprises:
(v) a pA,
wherein the components are operably linked to each other in a 5′-to-3′ direction as:
ITR1-P_{Euk}-hFXN-pA-ITR2.

64. The nucleic acid molecule of claim 63, wherein the pA site comprises the SV40 late polyadenylation site, the SV40 early polyadenylation site, the human β-globin polyadenylation site, or the bovine growth hormone polyadenylation site.

65. The nucleic acid molecule of claim 64, wherein the pA site comprises the SV40 late polyadenylation site.

66. The nucleic acid molecule of any one of claims 63-65, wherein the nucleic acid molecule further comprises:
(vi) an intron,
wherein the components are operably linked to each other in a 5′-to-3′ direction as:
ITR1-P_{Euk}-intron-hFXN-pA-ITR2.

67. The nucleic acid molecule of claim 66, wherein the intron is an SV40 intron.

68. The nucleic acid molecule of any one of claims 48-67, wherein the hFXN, or an RNA equivalent thereof, encodes a protein having an amino acid sequence that is at least 85% identical to the amino acid sequence of SEQ ID NO: 3.

69. The nucleic acid molecule of claim 68, wherein the hFXN, or an RNA equivalent thereof, encodes a protein having an amino acid sequence that is at least 95%, 96%, 97%, 98%, or 99% identical to the amino acid sequence of SEQ ID NO: 3.

70. The nucleic acid molecule of claim 69, wherein the hFXN, or an RNA equivalent thereof, encodes a protein having the amino acid sequence of SEQ ID NO: 3.

71. The nucleic acid molecule of any one of claims 48-70, wherein the hFXN, or an RNA equivalent thereof, has a nucleic acid sequence that is at least 85% identical to the nucleic acid of sequence of SEQ ID NO: 1.

72. The nucleic acid molecule of claim 71, wherein the hFXN, or an RNA equivalent thereof, has a nucleic acid sequence that is at least 90% identical to the nucleic acid of sequence of SEQ ID NO: 1.

73. The nucleic acid molecule of claim 72, wherein the hFXN, or an RNA equivalent thereof, has a nucleic acid sequence that is at least 95% identical to the nucleic acid of sequence of SEQ ID NO: 1.

74. The nucleic acid molecule of claim 73, wherein the hFXN, or an RNA equivalent thereof, has a nucleic acid sequence that is at least 97% identical to the nucleic acid of sequence of SEQ ID NO: 1.

75. The nucleic acid molecule of claim 74, wherein the hFXN, or an RNA equivalent thereof, has a nucleic acid sequence that is at least 98% identical to the nucleic acid of sequence of SEQ ID NO: 1.

76. The nucleic acid molecule of claim 75, wherein the hFXN, or an RNA equivalent thereof, has a nucleic acid sequence that is at least 99% identical to the nucleic acid of sequence of SEQ ID NO: 1.

77. The nucleic acid molecule of claim 76, wherein the hFXN, or an RNA equivalent thereof, has the nucleic acid sequence of SEQ ID NO: 1.

78. A vector comprising the nucleic acid molecule of any one of claims 48-77, optionally wherein the vector is a plasmid, a DNA vector, an RNA vector, a virion, or a viral vector.

79. The vector of claim 78, wherein the vector is a viral vector.

80. The vector of claim 79, wherein the viral vector is selected from the group consisting of an AAV, an adenovirus, a lentivirus, a retrovirus, a poxvirus, a baculovirus, a herpes simplex virus, a vaccinia virus, and a synthetic virus.

81. The vector of claim 80, wherein the viral vector is an AAV.

82. The vector of claim 81, wherein the AAV is comprises capsid proteins from an AAV serotype selected from the group consisting of AAV1, AAV2, AAV3, AAV4, AAV5, AAV6, AAV7, AAV8, AAV9, AAVrh10, and AAVrh74.

83. The vector of claim 81 or 82, wherein the viral vector is a pseudotyped AAV.

84. The vector of claim 83, wherein the pseudotyped AAV is AAV2/8 or AAV2/9, optionally wherein the pseudotyped AAV is AAV2/8.

85. The vector of any one of any one of claims 78-84, wherein ITR1 and/or ITR2 is a parvoviral ITR.

86. The vector of claim 85, wherein the parvoviral ITR is an AAV ITR.

87. The vector of claim 86, wherein the AAV ITR is an AAV serotype 2 ITR.

88. The vector of any one of claims 81-87, wherein the AAV comprises a recombinant capsid protein.

89. The vector of any one of claims 7-35 and 78-88, wherein the vector has a nucleic acid sequence that is at least 85% identical to the nucleic acid sequence of SEQ ID NO: 4.

90. The vector of claim 89, wherein the vector has a nucleic acid sequence that is at least 90% identical to the nucleic acid sequence of SEQ ID NO: 4.

91. The vector of claim 90, wherein the vector has a nucleic acid sequence that is at least 95% identical to the nucleic acid sequence of SEQ ID NO: 4, optionally wherein the vector has a nucleic acid sequence that is at least 96%, 97%, 98%, or 99% identical to the nucleic acid sequence of SEQ ID NO: 4.

92. The vector of claim 91, wherein the vector has the nucleic acid of SEQ ID NO: 4.

93. A plasmid encoding the viral vector of any one of claims 79-92.

94. The plasmid of claim 93, wherein the plasmid further comprises one or more SS, wherein the one or more SS is positioned 5′ to ITR1 and/or 3′ to ITR2.

95. The plasmid of claim 94, wherein the plasmid comprises two SS, wherein the two SS comprise a SS1 and a SS2, wherein SS1 is positioned 5′ to ITR1 and SS2 is positioned 3′ to ITR2.

96. The plasmid of claim 94 or 95, wherein the one or more SS does not comprise an open reading frame that is greater than 100 amino acids in length.

97. The plasmid of any one of claims 94-96, wherein the one or more SS does not comprise prokaryotic transcription factor binding sites.

98. The plasmid of any one of claims 95-97, wherein SS1 is about 1.0 Kb to about 5.0 Kb in length.

99. The plasmid of claim 98, wherein SS1 is about 2.0 Kb to about 5.0 Kb in length.

100. The plasmid of any one of claims 95-99, wherein SS2 is about 1.0 Kb to about 5.0 Kb in length.

101. The plasmid of claim 100, wherein SS2 is about 2.0 Kb to about 5.0 Kb in length.

102. The plasmid of any one of claims 95-101, wherein the plasmid further comprises a prokaryotic promoter operably linked to a selectable marker gene positioned 5′ to the one or more SS positioned 5′ to ITR1 or positioned 3′ to the one or more SS positioned 3′ to ITR2.

103. The plasmid of claim 102, wherein the selectable marker gene is an antibiotic resistance gene.

104. The plasmid of claim 102 or 103, wherein the plasmid further comprises a prokaryotic origin of replication positioned 5′ to the one or more SS positioned 5′ to ITR1 and/or positioned 3′ to the one or more SS positioned 3′ to ITR2.

105. A pharmaceutical composition comprising the nucleic acid molecule of any one of claims 48-77, the vector of any one of claims 78-92, or the plasmid of any one of claims 93-104 and a pharmaceutically acceptable carrier, diluent, or excipient.

106. A method of treating Friedreich Ataxia in a human patient in need thereof, the method comprising administering to the patient a therapeutically effective amount of the nucleic acid of any one of claims 1-6 and 48-77, the vector of any one of claims 7-35 and 78-92, the plasmid of any one of claims 36-46 and 93-104, or the pharmaceutical composition of claim 47 or 105.

107. A method of increasing frataxin expression in a human patient diagnosed as having Friedreich Ataxia, the method comprising administering to the patient a therapeutically effective amount of the nucleic acid of any one of claims 1-6 and 48-77, the vector of any one of claims 7-35 and 78-92, the plasmid of any one of claims 36-46 and 93-104, or the pharmaceutical composition of claim 47 or 105.

108. The method of claim 106 or 107, wherein the patient is from 3 years of age to 17 years of age.

109. The method of any one of claims 106-108, wherein upon administration of the nucleic acid, vector, plasmid, or pharmaceutical composition to the patient, the patient exhibits an increase in whole blood frataxin level, optionally wherein the patient exhibits the increase in whole blood frataxin level by about 12 weeks after administration.

110. The method of any one of claims 106-109, wherein upon administration of the nucleic acid, vector, plasmid, or pharmaceutical composition to the patient, the patient exhibits a reduction in Total Friedreich Ataxia Rating Scale Score, optionally wherein the patient exhibits the reduction in Total FARS Score by about 12 weeks after administration.

111. A kit comprising (i) the nucleic acid of any one of claims 1-6 and 48-77, the vector of any one of claims 7-35 and 78-92, the plasmid of any one of claims 36-46 and 93-104, or the pharmaceutical composition of claim 47 or 105, and (ii) a package insert, wherein the package insert instructs a user of the kit to administer the nucleic acid, vector, plasmid, or pharmaceutical composition to a human patient diagnosed as having Friedreich Ataxia.


SEQUENCE LISTING (6 sequences)

SEQ ID NO: 1 - DNA, 633 bp, synthetic construct
  source 1..633: mol_type = other DNA
atgtggaccctgggcaggagggctgtggctggcctgctggccagccccagccctgcccaggcccagaccctgaccagggtgcccaggcctgctgagctggcccccctgtgtggcaggaggggcctgaggactgacattgatgccacctgcacccccaggagggccagcagcaaccagaggggcctgaaccagatctggaatgtgaagaagcagtctgtgtacctgatgaacctgaggaagtctggcaccctgggccaccctggcagcctggatgaaaccacctatgagaggctggctgaggaaaccctggacagcctggctgagttctttgaggacctggctgacaagccctacacctttgaggactatgatgtgagctttggctctggggtgctgactgtgaagctggggggggacctgggcacctatgtgatcaacaagcagacccccaacaagcagatctggctgagcagccccagctctggccccaagagatatgactggactggcaagaactgggtgtacagccatgatggggtgagcctgcatgagctgctggctgctgagctgaccaaggccctgaagaccaagctggacctgagcagcctggcctactctggcaaggatgcctga

SEQ ID NO: 2 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 3 - protein, 210 aa, synthetic construct
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 4 - DNA, 9224 bp, synthetic construct
  source 1..9224: mol_type = other DNA
tcgcgcgtttcggtgatgacggtgaaaacctctgacacatgcagctcccggacgtcattgtcgatcctgcaggcgtacgccatccaatggaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggtttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacaagatttctattttaattggattcttagggctaataaaataatcagccttagcacttatttatttattttttttgagagggagtctcgctctgttgtccatgctggagtgcagtggcgtgatctcggctcactgcaagctccacctcatgagttcacaccattctcctgcctcagtctcccgagtagctgggactccaggcgccctctacaaagcccgtctaattttttttgtatttttagtagagacagggtttcactgtgttagccaggatggtcttgatctcctgaccttgtgatctgcccgcctcggcctcccaaagtgctgggattataggcttgagccactgctcccggccagcacttatttttataattcttcatgattactgtgttactgtcccatgggccgccagggccagctaggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctagcacgcgtagaatcagaaaatctgagaagaaaacttaaaaacctttgaatctaacctactcaaataaactttgaatatatttattgaaatatttaatttgtttattttttatttttttattttagagacaaggtctcactaaagtgcaggctagagtgcagtgacacaatcatggctatggctcactgcaacctcaaactcctgggctcaagcgattctgttgccttaggttcgccaggagctggcactacaggtgccaccacacctggcttttttgttttgttttttttgggtagagaaggggatttggtatgttgcttaggctgatcttgaactagcctcacgcaatcctgcttcggctttccaaaatgttgggattataggcatgagccatgcgcctgaccttgattacctcgatgatgtgtatgtcatacattggagggcaaagacatctctgaattcctcacaacagccatggcagggggcaggtacccatgttacagatgacagactgatgcatagagaagttaagtcgtggggagtttactttctcctaaattgtcctgttactagatgaatttgtttttgtttcattttgttttgtttttgagacagagtctcatgttgtcacccagactggagtgcagtggctccatctcggctcactatagcctccgcctcctgagttcaagtgattctcctgcctcagcctcccaagtagctgggtgcatgccaccacgcctggctaatttttgtatttttagtatagatggggtttcaccatgttggccagactggtctcgaactcctgacctcaagtgatccaccccgcgcttagcctcccaaagtgctgggattataggcgtgagccaccacaccaggccatgaatttgttttcaatatttatttattttgtattttctatttttgagatggagtctcgctctgctgtccaggctagagtgcagtggtgtgatcttggctcactgtagcctccacctcctgagttcaagtgattctcctgccttagcctcccgagtagctgggattacaggcgcccaccatcacacccggctaatttttgtatttttagtagagatggggtttcaccatgttggccacgctggtctcaaactgacctcaattgatccacccaccttgacctcccaaagtgctgggattacagacctgagccacagcgcccagcccttcaatatttatttaaatttgcctgctggctaacttctcattgcacctgggctctagtgtaattaaattacttcattctctttttaaaactttttacttttttcttttttgtgtttttcattctcttatctacgagagccacaatacttgaagacaccaattgataccccttagtcacatctgagctaaacactttcagttcctacagctgtttcttaatcttaggtcacatggtttcttcccatgctgttcttcccagacagcattttttttttttttgagagtctcactctgttgcccaggctggagtacagtagcacaatctcagtttactgcaacctctgccttccaggttcaggtgattctcctgcttcagcctcctgagtagctgggactacaggagcgtgccaccacgcccggctaatttttgtatttttagtagagacagggtttcaccatgttggccaggctggtctcgaactccttaccttgtgatccgcctgtctcggcctcccaaagtggtgggattacaggtgtgagccaccacgcctggttcttacatttattttggaataaatttagatacacagaaaagttgcaaagataagagtttccatataaccctcacccagtttgccttccctaatgttcgcgtggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctctaaggtaaatataaaatttttaagtgtataatgtgttaaactactgattctaattgtttctctcttttagattccaacctttagaactgaccaccatgtggaccctgggcaggagggctgtggctggcctgctggccagccccagccctgcccaggcccagaccctgaccagggtgcccaggcctgctgagctggcccccctgtgtggcaggaggggcctgaggactgacattgatgccacctgcacccccaggagggccagcagcaaccagaggggcctgaaccagatctggaatgtgaagaagcagtctgtgtacctgatgaacctgaggaagtctggcaccctgggccaccctggcagcctggatgaaaccacctatgagaggctggctgaggaaaccctggacagcctggctgagttctttgaggacctggctgacaagccctacacctttgaggactatgatgtgagctttggctctggggtgctgactgtgaagctggggggggacctgggcacctatgtgatcaacaagcagacccccaacaagcagatctggctgagcagccccagctctggccccaagagatatgactggactggcaagaactgggtgtacagccatgatggggtgagcctgcatgagctgctggctgctgagctgaccaaggccctgaagaccaagctggacctgagcagcctggcctactctggcaaggatgcctgagatatcgctttatttgtgaaatttgtgatgctattgctttatttgtaaccattataagctgcaataaacaagttaacaacaacaattgcattcattttatgtttcaggttcagggggaggtgtgggaggttttttaaagcggccaacatcttacattatcatggtacatttgtcaaaactaagacacttttttttctaataaaaataatagagatgaggtctcactatattgtccaggctggtctcaaactttgagctcaagcagtcctcccacctccacctcccaaagtgctgggattacaggcatgaaccaccacacccagcctacattgttatgttactactctccagactattcagatttcaccaatttttctatccatagcctttttttctgtttcaggatccaattcaggacaccataccactgtatggttaattttaaaaactcaaatgttgggagtttgctttatcaccgctgtatcttatcatctcgttagataattttgtatctggagcctgctctctagcatgaataagtaaaaatgtggactttgtgatttacagatctgatatttatgtttttgtgtttttactttaagtactagacaaagtaaatctaagaggtctgtggtactgcactagagaattgttacttgattatggtgggccaagtggttgaaaatttacctaacaatactataacataggccatatttcataattttaatcacaaggcaattgtgaaaattttatcaaatgtttaataaaagcaaggtgaagaaggtgatagctttaaatttacttgccatttttgggcacacaaaagtaatttgctctgccacttagagttataaggtcaaagtgggagtaaataatctttgtattaggaatgcggccgctctaggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacctagaggccgccagggccatatttctcaatttttaaatttttcaaaaaaattaatccttaatgtgcatatttttgaattgttaatataactttttgaggtgatgtcttcatgtgtttcaactacttaaaaacttttaaacagtatataataaaaaatcttccaggccactcacacctgtaatcccagcactttgggaggctgaggtgggcagatcacctgagggcaggagttcgagaccagcctggccaatatatatatattcatatattcatatatatatatatattcatatattcatatatatatattcatatattcatatatatatatatatatatatatagcaaaacctcatctctaataaaatacaaaaattagctgagcgtggtgatggatgcctgtagtcccagctactcgggaggctgaggcaggagaatctcttgaacctgggaggtggaggttgcagtgagctgagatggtgccactgccctccagcctgagtgacagagcgagactcggtctccaaaaaaaaacaacaaaaaaatcttccatccttgtctcccatccaccccttccccccagcatgtacttgcagactttatgcatatacagtgagtactgtatatacacaaataataaaaaaatcatatatataatatatgtaattcccctttacatgaaaggtagcacactggtctgtacagtctgtctgcactgtgctatttcactttatatttttatagtttgacagagttctaacatttctttttttttttttttaacagagtcttgttcctgattgttaaattttaaagcatcctaaagtttggtttcacacttgaatgaataccatgtaaggattcacttacatagatgtggttgcctgaatcttaagaataaaataacattgtttgtatttatttaaattagtgttccttttatggtttgcctgaaagcacaacaaaatcctcaccaagatattacaattatgactcccatacaggtaaactgtttagagattggcaagcaccttttaatgaaaggagtcagccagcttagtgtgcagtatttatttctgccggaagagggagcttcagggacagactttggtttagtcatgaagcctccagcactcccaagcggttgtggttgaccaagcaatttatgcttttacctttctacttccagaggcttgtttacttatcagtaagcattaatttagtgtcccctcagatgccttttactttcttcttttctgcctagaataagctgctcttccaattttgcagctacatgtttccaccccagttggaatttctccataacatccattgtagctatccttcaatctacagcctctatttcctgttatagctggtcaggtctaatccctcaaaatactctgtcccctgcttcccttatctgctggccacctttttcccccacatacacactgccatgtcccacccttcactcaagttgttccctgccacctcaacaaatttaagtccataaaaccatccaatgggctcgagccctgcaggatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 5 - DNA, 7168 bp, synthetic construct
  source 1..7168: mol_type = other DNA
agtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcatgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgatgcccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttgtattaatatgtagcaaaggcttttccaatgggtgaataaaaacacattccattaagtcaagctgggagcagtggcatatacctatagtcccagctgcacaggaggctgagacaggaggattgcttgaagccaggaattggagatcagcctgggcaacacagcaagatcctatctcttaaaaaaagaaaaaaaaacctattaataataaaacagtataaacaaaagctaaataggtaaaatattttttctgaaataaaattattttttgagtctgatggaaatgtttaagtgcagtaggccagtgccagtgagaaaataaataacatcatacatgtttgtatgtgtttgcatcttgcttctactgaaagtttcagtgcaccccacttacttagaactcggtgacatgatgtactcctttatctgggacacagcacaaaagaggtatgcagtggggctgctctgacatgaaagtggaagttaaggaatctgggctcttatggggtccttgtgggccagcccttcaggcctattttactttcattttacatatagctctaattggtttgattatctcgttcccaaggcagtgggagatccccatttaaggaaagaaaaggggcctggcacagtggctcatgcctgtaatcccagcactttgggaggctgaggcaagtgtatcacctgaggtcaggagttcaagaccagcctggccaacatggcaaaatcccgtctctactaaaaatattaaaaaattggctgggcgtggtggttcgtgcctataatttcagctactcaggaggctgaggcaggagaatcgctgtaacctggggggtggaggttgcagtgagacgagatcatgccacttcactccagcctggccaacagagccatactccgtctcaaataaataaataaataaataaagggacttcaaacacatgaacagcagccaggggaagaatcaaaatcatattctgtcaagcaaactggaaaagtaccactgtgtgtaccaatagcctccccaccacagaccctgggagcatcgcctcatttatggtgtggtccagtcatccatgtgaaggatgagtttccaggaaaaggttattaaatattcactgtaacatactggaggaggtgaggaattgcataatacaatcttagaaaacttttttttcccctttctattttttgagacaggatctcactttggcactcaggctggaggacagtggtacaatcaaagctcatggcagcctcgacctccctgggcttgggcaatcctcccacaggtgtgcacctccatagctggctaatttgtgtattttttgtagagatggggtttcaccatgttgcccaggctggtctctaacacttaggctcaagtgatccacctgcctcgtcctcccaagatgctgggattacaggtgtgtgccacaggtgttcatcagaaagctttttctattatttttaccttcttgagtgggtagaacctcagccacatagaaaataaaatgttctggcatgacttatttagctctctggaattacaaagaaggaatgaggtgtgtaaaagagaacctgggtttttgaatcacaaatttagaatttaatcgaaactctgcctcttacttgtttgtagacactgacagtggcctcatgttttttttttttttaatctataaaatggagatatctaacatgttgagcctgggcccacaggcaaagcacaatcctgatgtgagaagtactcagttcatgacaactgttgttctcacatgcatagcataatttcatattcacattggaggacttctcccaaaatatggatgacgttccctactcaaccttgaacttaatcaaaatactcagtttacttaacttcgtattagattctgattccctggaaccatttatcgtgtgccttaccatgcttatattttacttgatcttttgcataccttctaaaactattttagccaatttaaaatttgacagtttgcattaaattataggtttacaatatgctttatccagctatacctgccccaaattctgacagatgcttttgccacctctaaaggaagacccatgttcatagtgatggagtttgtgtggactaaccatgcaaggttgccaaggaaaaatcgctttacgcttccaaggtacacactaagatgaaagtaattttagtccgtgtccagttggattcttggcacatagttatcttctgctagaacaaactaaaacagctacatgccagcaagggagaaaggggaaggaggggcaaagttttgaaatttcatgtaaatttatgctgttcaaaacgacgagttcatgactttgtgtatagagtaagaaatgccttttcttttttgagacagagtcttgctctgtcacccaggctggagtgcagtggcacgatctgggctcactacaacctccgcctcctgggttcaagcaattctctgcctcagcctcccgagtagctgggattacaggtgcctgccaccacacccggctaatttttgtatttttagtagagacggggtttcaccatcatggccaggctggtcttgaactcctgacctagtaatccacctgcctccgcctcccaaagtgctgggattacaggcgtgagccactgcacccagccagaaatgccttctaatctttggtttatcttaattagccaggacacttggagtgcatcccgaagtacctgatcagtggcccctttggaatgtgtaaaactcagctcacttatatccctgcatccgctacagagacagaatccaagctcatatgttccatcttctctggctgtatagtttaaggaatggaaggcaccagaacagatttattgaaatgtttattagctgaagatttatttagacagttgaggaaaacatcagcacccagcagtaaaattggctctcaaagattttcttctcctgtggaaagtcagacctctgaggccccatccaggtagaagtactagtgcaagaagggcctctgctgtccacttgtgtttctgtgatctgtgggaacattgttaacgccacatcttgacctcaaattgtttagctcctggccagacacggtggctcacacctgtaatcccagcactttgagaggctgaggcaggtggatcacctgaggttaggagttcgaggccagcctggtcaacatggtaaaaccccgcctctactaaaaatacaaaaattagctggccgtagtggcgcacgcctgttatcccagctactcgggaggctgaggcaggagaattgcttgaacctgggtggtggaggttgcagtgagccgagattacaccactgcactccagcctgggtgacaagagggaaactccattaaaaaaatgtaattcccgtgtctgccatcttaagtgtaaaggtggctaaattatatagaaaaataagacaatatcatttcccaattacattcctttcctaccgcactctatgatgctagctgagatttttccaaaagaaaatggcttaaataaaaccctaagagaaagaaaaactttaaatccctccaaagctcaaaagtaatagaaacagatgagtttggagtcaggatttctctgtaagattgcctaggctgtgtactgcacatctccaggtgccactgttgacagagattataactacaatgtgaagtgaatggtgccactgacagttatgcaaaccgtccagagcatagccacctgatcctgctgggattcctcttgccagtccatcagcagttccccttgaaagtttcaccaaacatcccttaaatctgccctctcctgcccgtccccagtggaggtcctcatcatttttcacctgcatttttgcaggagctttcttatatccaccttcctccttttctctcagcccatcatctagctacacagtctccagggtaagctttcagaaaggcaatctcttgtctgtaaaacctaagcaggaccaaggccaagtttcttagcctgaaaaatgtgcttttctgactgaactgttcaggcactgactctacatataattatgcttttctaccccctcacactcaacactttgactccagcaatcccaaatccccagatccctaagtgtgctgtgctattttcacgtggctctcagacttggccagtgctgtttccattttggtctttattccccacatctctgcctggggggtagattctaccctgaaaaatgttcttggcacagccttgcaaactcctcctccactcagcctctgcctggatgcccttgattgttccatgtcctcagcataccatgtttgtctttcccagcactgacctaccatgtgtcacccctgcttggctgtaccttccatgaggctaggactatgtgtctcctttgttgactgctgttgccctagcatcttgcacagttccttgcacacaattagagctctataaatgtcaaataaatgtgttataattatatgtttaagatagttgttcaaataaactctaaataaccccaac

SEQ ID NO: 6 - skipped